(19) United States
(12) Patent Application Publication
(10) Pub. No.: US 2025/0344566 A1
(43) Pub. Date: Nov. 6, 2025

(54) ORGANIC ELECTROLUMINESCENT ELEMENT

(71) Applicant: TOSOH CORPORATION, Yamaguchi (JP)

(72) Inventors: Naoki UCHIDA, Kanagawa (JP); Fuminari UEHARA, Kanagawa (JP); Kana OIKE, Kanagawa (JP); Yasuhiro TAKAHASHI, Kanagawa (JP); Shintaro NOMURA, Kanagawa (JP); Eriko OOTA, Kanagawa (JP)

(73) Assignee: TOSOH CORPORATION, Yamaguchi (JP)

(21) Appl. No.: 18/879,063

(22) PCT Filed: Jun. 29, 2023
(86) PCT No.: PCT/JP2023/024112

§ 371 Date: Dec. 26, 2024

(30) Foreign Application Priority Data
Jul. 1, 2022 (JP) 2022-106807
Jul. 1, 2022 (JP) 2022-106810
May 30, 2023 (JP) 2023-088821
May 30, 2023 (JP) 2023-088916

(51) Int. Cl. H10K 50/11; C09K 11/06; H10K 50/15; H10K 50/16; H10K 85/40; H10K 85/60

(52) U.S. Cl. CPC H10K 50/11; C09K 11/06; H10K 50/157; H10K 50/16; H10K 85/40; H10K 85/626; H10K 85/633; H10K 85/636; H10K 85/654; H10K 85/656; H10K 85/6572; H10K 85/6574; H10K 85/6576; H10K 85/658; C09K 2211/1059

(57) ABSTRACT

An organic electroluminescent element includes a positive electrode, a negative electrode opposite the positive electrode, a light-emitting layer disposed between the positive electrode and the negative electrode and a hole transport region disposed between the positive electrode and the light-emitting layer. The organic electroluminescent element includes an electron transport region disposed between the light-emitting layer and the negative electrode, the electron transport region contains a triazine compound represented by formula (2), and the organic electroluminescent element satisfies at least one or more of condition (i) or (ii) below.
(i) The light-emitting layer contains a boron compound represented by formula (1).
(ii) The hole transport region contains a radialene compound represented by formula (3).

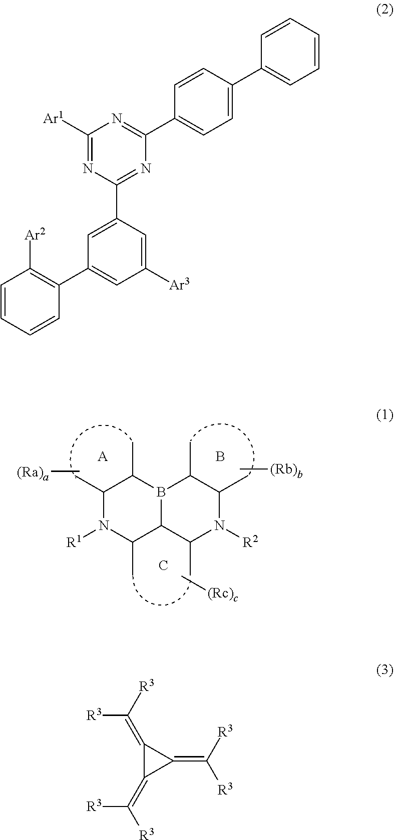

### TECHNICAL FIELD

The present disclosure relates to an organic electroluminescent element.

### BACKGROUND ART

From organic electroluminescent elements, light of any wavelength can be extracted by using appropriate light-emitting materials. Because of their usefulness, organic electroluminescent elements have been applied and extended not only to small-sized display equipment but also to applications such as lighting, resulting in active development underway. In all applications, any color tone can be reproduced by combining light in the colors of red, green and blue at appropriate intensities.

In the related art, it has been desired that organic electroluminescent elements operate at low voltage and emit light with high efficiency. Organic electroluminescent element in recent years, therefore, have been disclosed.

In the related art, it has been desired that organic electroluminescent elements operate at low voltage and emit light with high efficiency. Materials for organic electroluminescent elements in recent years, therefore, have been gradually improved.

For example, PTL 1 and PTL 2 disclose organic electroluminescent elements with low drive voltage, high efficiency and long life.

### CITATION LIST

### Patent Literature

PTL 1: International Publication No. 2020/117026
PTL 2: Japanese Unexamined Patent Application Publication No. 2020-033264

### SUMMARY OF INVENTION

### Technical Problem

The market's requirement for organic electroluminescent elements in recent years, however, has been increasingly high; there is a need for the development of organic electroluminescent elements that not only have superior drive voltage characteristics and current efficiency characteristics but also support precise control of color tone. To support fine tuning and control of color tone, furthermore, high efficiency, even across a broad range from low luminance to high luminance, combined with a small change in efficiency is desired (hereinafter, this type of change in efficiency is also referred to simply as “efficiency change”). Moreover, for further fine tuning and control of color tone to be enabled, it is desired that no change in efficiency occur between before and after continuous operation of the element (hereinafter, this type of change in efficiency is also referred to simply as “efficiency change after continuous operation”)

An aspect of the present disclosure is directed to the provision of an organic electroluminescent element that combines superior drive voltage characteristics and current efficiency characteristics and achieves a small efficiency change and a small efficiency change after continuous operation.

### Solution to Problem

An organic electroluminescent element according to an aspect of the present disclosure is:

[1]

An organic electroluminescent element comprising:
a positive electrode;
a negative electrode opposite the positive electrode;
a light-emitting layer disposed between the positive electrode and the negative electrode; and
a hole transport region disposed between the positive electrode and the light-emitting layer, wherein:
the organic electroluminescent element includes an electron transport region disposed between the light-emitting layer and the negative electrode;
the electron transport region contains a triazine compound represented by formula (2); and
the organic electroluminescent element satisfies at least one or more of condition (i) or (ii) below.
(i) The light-emitting layer contains a boron compound represented by formula (1).
(ii) The hole transport region contains a radialene compound represented by formula (3).

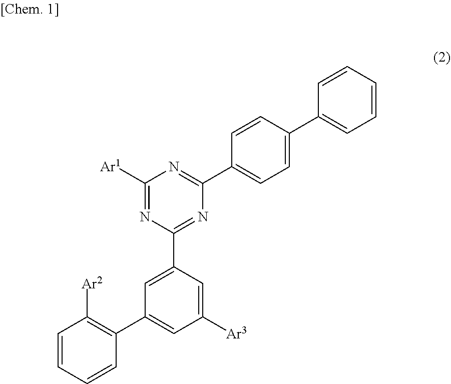

In formula (2),
Ar¹ represents a phenyl group, naphthyl group or biphenylyl group optionally substituted with a C1 to C6 alkyl group, and
Ar² represents a phenyl group, pyridyl group, phenylpyridyl group, pyridylphenyl group or biphenylyl group optionally substituted with a C1 to C4 alkyl group.
Ar³ represents a phenyl group or biphenylyl group optionally substituted with a C1 to C4 alkyl group.

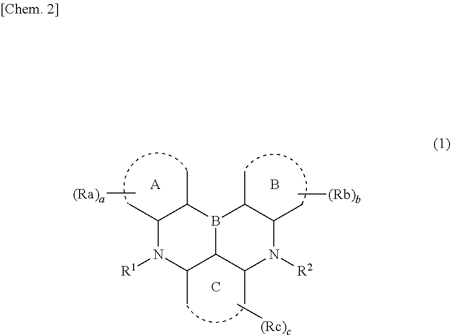

In formula (1),
ring A, ring B and ring C each independently represent
an aromatic hydrocarbon ring in which the number of ring-constituting atoms is from 6 to 20 or
an aromatic heterocycle in which the number of ring-constituting atoms is from 5 to 20,
each optionally having a substituent;
Ra, Rb and Rc each independently represent
   deuterium,
   a cyano group,
   a C1 to C20 alkyl group,
   a substituted or unsubstituted amino group,
   an aromatic hydrocarbon group in which the number of ring-constituting atoms is from 6 to 20 or
   a heteroaromatic group in which the number of ring-constituting atoms is from 5 to 20;
each of a, b and c is independently an integer of 0 to 4,
ring A and ring B are optionally linked together via an oxygen atom, a sulfur atom, N—R′, Ra or Rb,
R¹, R² and R′ each independently represent
   a C1 to C20 alkyl group,
   an aromatic hydrocarbon group in which the number of ring-constituting atoms is from 6 to 20 or
   a heteroaromatic group in which the number of ring-constituting atoms is from 5 to 20,
   each optionally having a substituent; and
R¹ optionally forms a ring structure by being linked to ring A or ring C via an oxygen atom, a sulfur atom, a substituted or unsubstituted nitrogen atom, Ra or Rc, and
R² optionally forms a ring structure by being linked to ring B or ring C via an oxygen atom, a sulfur atom, a substituted or unsubstituted nitrogen atom, Rb or Rc.

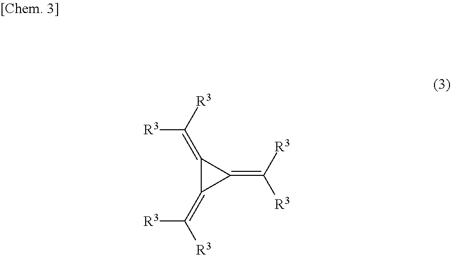

In formula (3),
R³ represents a cyano group, a halogen atom, a fluoroalkyl group, an acyl group, a sulfonyl group, a phosphoryl group, a substituted or unsubstituted aromatic hydrocarbon group or a substituted or unsubstituted aromatic heterocyclic group, independently at each occurrence.
   [2]

The organic electroluminescent element according to [1], wherein the organic electroluminescent element satisfies (i) of the conditions (i) and (ii).

[3]

The organic electroluminescent element according to [1], wherein the organic electroluminescent element satisfies (ii) of the conditions (i) and (ii).

[4]

The organic electroluminescent element according to [1], wherein the organic electroluminescent element satisfies both (i) and (ii) of the conditions (i) and (ii).

[5]

The organic electroluminescent element according to [2] or [4], wherein in formula (1), at least one of rings A to C is a benzene ring optionally having a substituent.

[6]

The organic electroluminescent element according to [2], [4] or [5], wherein in formula (1), rings A to C are ring systems containing a benzene structure optionally having a substituent.

[7]

The organic electroluminescent element according to [2], [4], [5] or [6], wherein in formula (1), at least one of Ra, Rb or Rc is a C1 to C4 alkyl group.

[8]

The organic electroluminescent element according to [2], [4], [5], [6] or [7], wherein the boron compound represented by formula (1) is selected from the following.

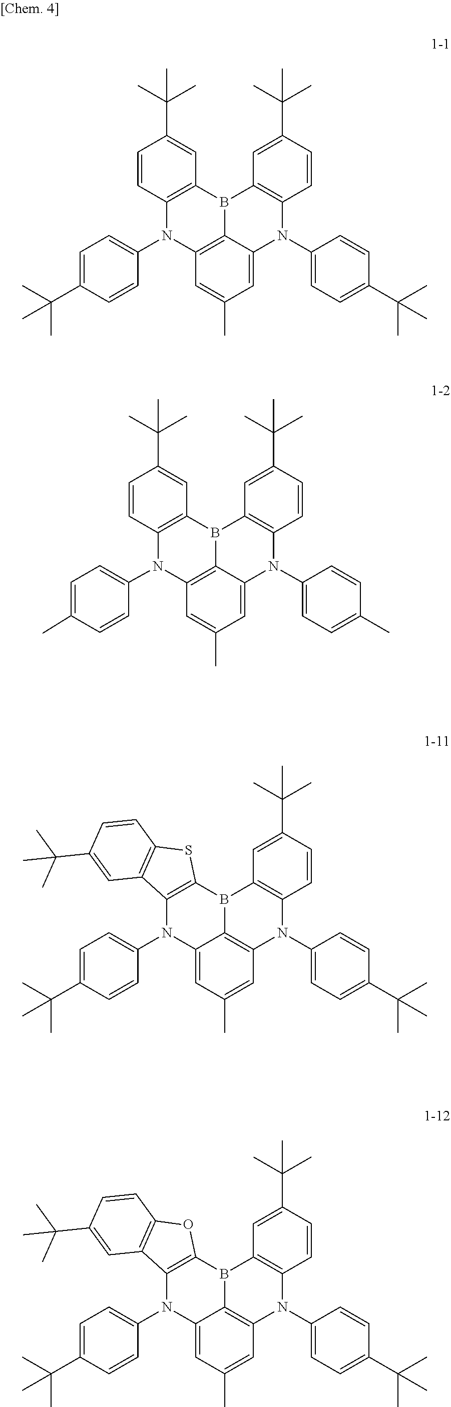

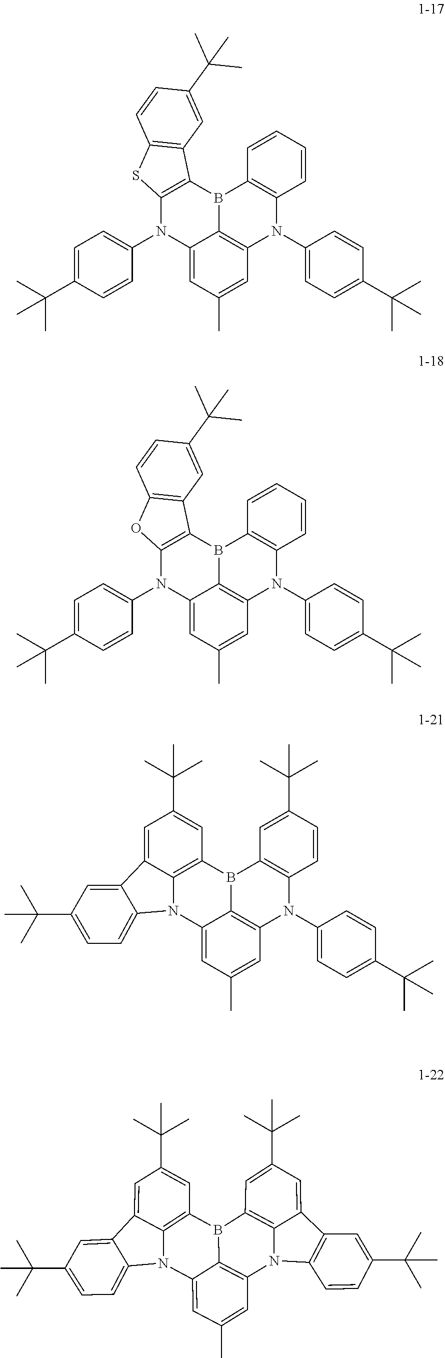

[9]

The organic electroluminescent element according to [2], [4], [5], [6], [7] or [8], wherein the boron compound represented by formula (1) is the compound of 1-1 above.

[10]

The organic electroluminescent element according to [3] or [4], wherein the hole transport region includes at least a first hole transport layer and
a second hole transport layer disposed between the first hole transport layer and the light-emitting layer, and
the first hole transport layer contains the radialene compound represented by formula (3).
   [11]

The organic electroluminescent element according to [3], [4] or [10], wherein the radialene compound represented by formula (3) is formula (3-a).

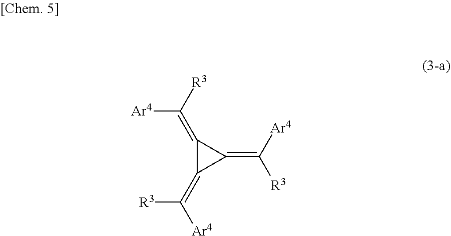

In formula (3-a),
R³ represents a cyano group, a halogen atom, a fluoroalkyl group, an acyl group, a sulfonyl group, a phosphoryl group, a substituted or unsubstituted aromatic hydrocarbon group or a substituted or unsubstituted aromatic heterocyclic group, independently at each occurrence.
Ar⁴ represents a substituted or unsubstituted aromatic hydrocarbon group or a substituted or unsubstituted aromatic heterocyclic group, independently at each occurrence.

Each substituent that a substituted or unsubstituted aromatic hydrocarbon group or substituted or unsubstituted aromatic heterocyclic group represented by R³ and Ar⁴ has is independently selected from
deuterium, a cyano group, a halogen atom, a fluoroalkyl group, an acyl group, a sulfonyl group and a phosphoryl group.
   [12]

The organic electroluminescent element according to [3], [4], or [11], wherein the radialene compound represented by formula (3) is formula (3-b).

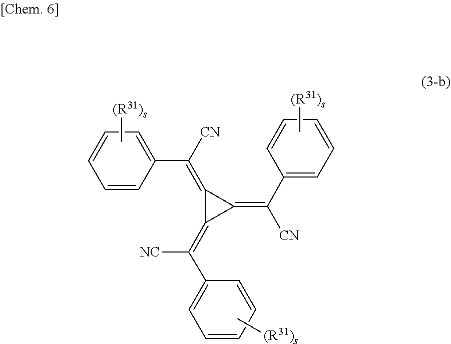

In formula (3-b),
R³¹ is selected from deuterium, a cyano group, a halogen atom, a fluoroalkyl group, an acyl group, a sulfonyl group and a phosphoryl group, independently at each occurrence.
s is an integer of 1 to 5, independently at each occurrence.
   [13]

The organic electroluminescent element according to [3], [4], [10], or [12], wherein the radialene compound represented by formula (3) is formula 3-1 below.

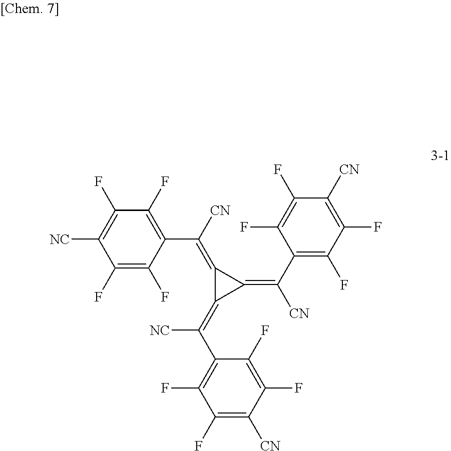 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

[14]

The organic electroluminescent element according to [1] to [13], wherein the electron transport region includes at least a first electron transport layer and a second electron transport layer disposed between the first electron transport layer and the negative electrode, and
the second electron transport layer contains the triazine compound represented by formula (2).
   [15]

The organic electroluminescent element according to [1] to [13], wherein the triazine compound represented by formula (2) is the following.

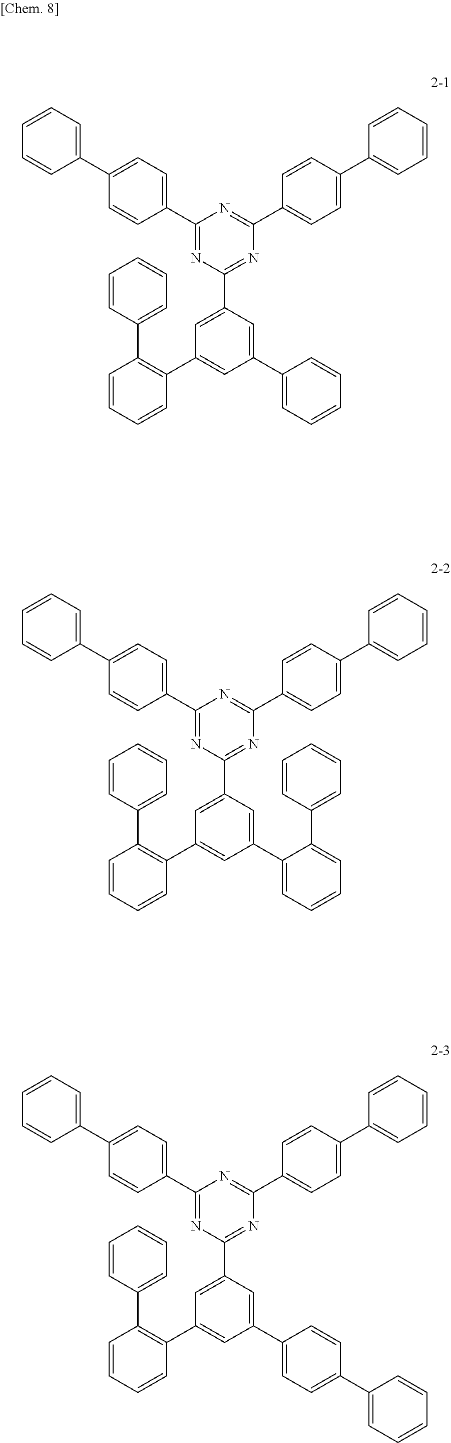 [SMILES: c1ccc(-c2ccc(-c3nc(-c4ccc(-c5ccccc5)cc4)nc(-c4cc(-c5ccccc5)cc(-c5ccccc5-c5ccccc5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5ccccc5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4ccccc4)c3)cc2)cc1]

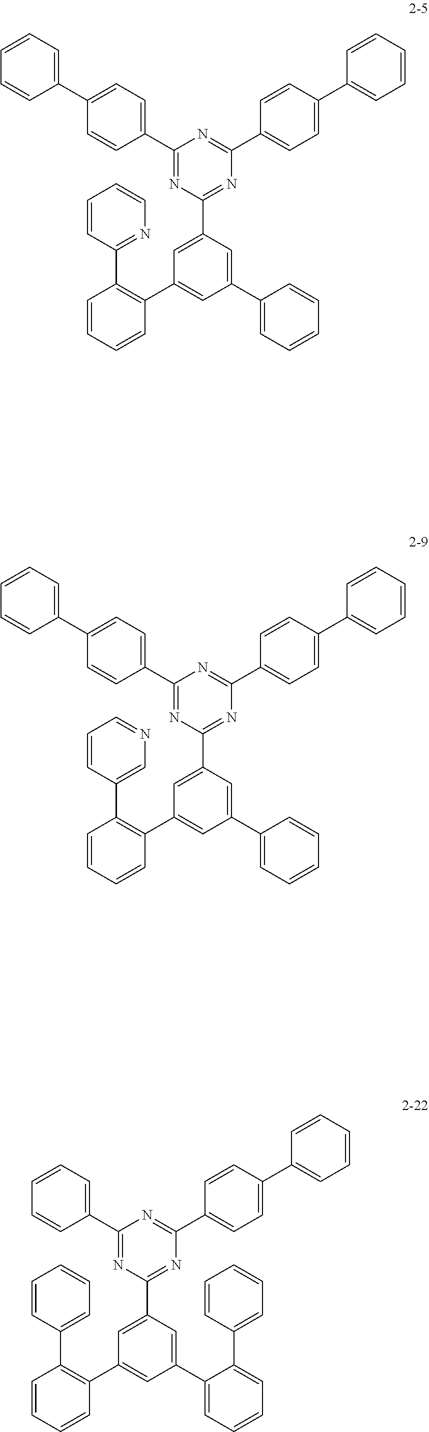 [SMILES: c1ccc(-c2ccc(-c3nc(-c4ccc(-c5ccccc5)cc4)nc(-c4cc(-c5ccccc5)cc(-c5ccccc5-c5ccccn5)c4)n3)cc2)cc1 ; c1ccc(-c2ccc(-c3nc(-c4ccc(-c5ccccc5)cc4)nc(-c4cc(-c5ccccc5)cc(-c5ccccc5-c5cccnc5)c4)n3)cc2)cc1 ; c1ccc(-c2ccc(-c3nc(-c4ccccc4)nc(-c4cc(-c5ccccc5-c5ccccc5)cc(-c5ccccc5-c5ccccc5)c4)n3)cc2)cc1]

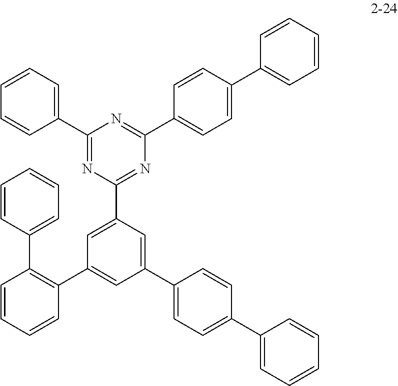 [SMILES: c1ccc(-c2ccc(-c3cc(-c4nc(-c5ccccc5)nc(-c5ccc(-c6ccccc6)cc5)n4)cc(-c4ccccc4-c4ccccc4)c3)cc2)cc1]

[16]

The organic electroluminescent element according to [1] to [15], wherein the second electron transport layer contains the compound represented by formula (2) and lithium quinolate (Liq).

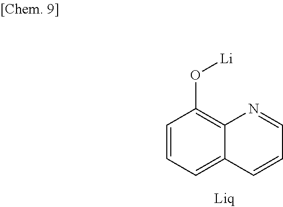

### Advantageous Effects of Invention

An organic electroluminescent element that is an aspect of the present disclosure combines superior drive voltage characteristics and current efficiency characteristics. With this element, furthermore, the efficiency change and the efficiency change after continuous operation can be reduced, and an element suitable for the reproduction of color tones for display devices and lighting devices can be achieved.

### BRIEF DESCRIPTION OF DRAWINGS

FIG. 1 is a schematic cross-sectional view illustrating an example of a layer configuration of an organic electroluminescent element according to an aspect of the present disclosure.

### DESCRIPTION OF EMBODIMENTS

In the following, an organic electroluminescent element according to an aspect of the present disclosure will be described in detail.

### <Organic Electroluminescent Element>

An organic electroluminescent element according to an aspect of the present disclosure is
an organic electroluminescent element comprising:
a positive electrode;
a negative electrode opposite the positive electrode;
a light-emitting layer disposed between the positive electrode and the negative electrode; and
a hole transport region disposed between the positive electrode and the light-emitting layer, wherein:
the organic electroluminescent element includes an electron transport region disposed between the light-emitting layer and the negative electrode;
the electron transport region contains a triazine compound represented by formula (2); and
the organic electroluminescent element satisfies at least one or more of condition (i) or (ii) below.
(i) The light-emitting layer contains a boron compound represented by formula (1).
(ii) The hole transport region contains a radialene compound represented by formula (3).

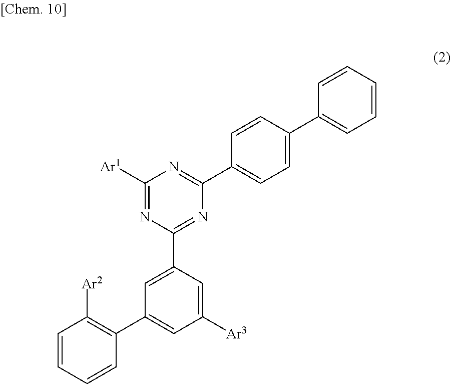

In formula (2),
Ar¹ represents a phenyl group, naphthyl group or biphenylyl group optionally substituted with a C1 to C6 alkyl group, and
Ar² represents a phenyl group, pyridyl group, phenylpyridyl group, pyridylphenyl group or biphenylyl group optionally substituted with a C1 to C4 alkyl group.
Ar³ represents a phenyl group or biphenylyl group optionally substituted with a C1 to C4 alkyl group.

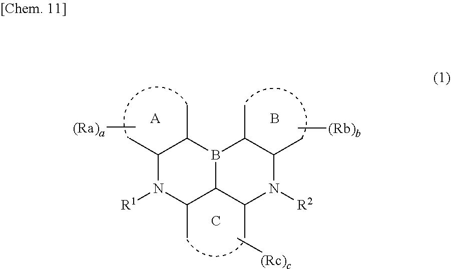

In formula (1),
ring A, ring B and ring C each independently represent
an aromatic hydrocarbon ring in which the number of ring-constituting atoms is from 6 to 20 or
an aromatic heterocycle in which the number of ring-constituting atoms is from 5 to 20,
each optionally having a substituent;
Ra, Rb and Rc each independently represent
   deuterium,
   a cyano group,
   a C1 to C20 alkyl group,
   a substituted or unsubstituted amino group,
   an aromatic hydrocarbon group in which the number of ring-constituting atoms is from 6 to 20 or
   a heteroaromatic group in which the number of ring-constituting atoms is from 5 to 20;
each of a, b and c is independently an integer of 0 to 4,
ring A and ring B are optionally linked together via an oxygen atom, a sulfur atom, N—R′, Ra or Rb,
R¹, R² and R′ each independently represent
   a C1 to C20 alkyl group,
   an aromatic hydrocarbon group in which the number of ring-constituting atoms is from 6 to 20 or
   a heteroaromatic group in which the number of ring-constituting atoms is from 5 to 20,
   each optionally having a substituent; and

R¹ optionally forms a ring structure by being linked to ring A or ring C via an oxygen atom, a sulfur atom, a substituted or unsubstituted nitrogen atom, Ra or Rc, and
R² optionally forms a ring structure by being linked to ring B or ring C via an oxygen atom, a sulfur atom, a substituted or unsubstituted nitrogen atom, Rb or Rc.

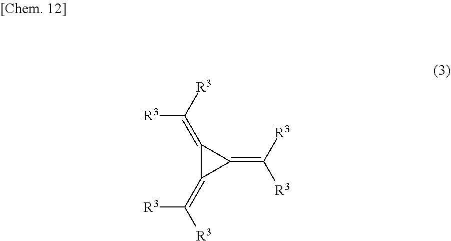

In formula (3),
R³ represents a cyano group, a halogen atom, a fluoroalkyl group, an acyl group, a sulfonyl group, a phosphoryl group, a substituted or unsubstituted aromatic hydrocarbon group or a substituted or unsubstituted aromatic heterocyclic group, independently at each occurrence.

### [Structure of the Organic Electroluminescent Element]

The region between the positive electrode and the light-emitting layer is defined as a hole transport region. The hole transport region has the function of transmitting holes injected from the positive electrode or holes generated inside the hole transport region to the light-emitting layer. Interposing this hole transport region between the positive electrode and the light-emitting layer leads to the injection of more holes into the light-emitting layer with a lower electric field.

The hole transport region may be formed by multiple layers. The hole transport region is preferably in a multilayer structure including two or more layers, more preferably in a multilayer structure including four or fewer layers, particularly preferably in a three-layer multilayer structure. When the hole transport region forms a three-layer multilayer structure, the layers are defined as a first hole transport layer, a second hole transport layer and a third hole transport layer, in order from the positive electrode side. Preferably, these layers each independently have the function(s) of one or more selected from the group consisting of a hole injection layer, a hole-generating layer, a hole transport layer and an electron-blocking layer.

The region between the negative electrode and the light-emitting layer is defined as an electron transport region. The electron transport region has the function of transmitting electrons injected from the negative electrode or electrons generated inside the electron transport region to the light-emitting layer. Interposing this electron transport region between the negative electrode and the light-emitting layer leads to the injection of more electrons into the light-emitting layer with a lower electric field.

The electron transport region may be formed by multiple layers. The electron transport region is preferably in a multilayer structure including two or more layers, more preferably in a multilayer structure including four or fewer layers, particularly preferably in a multilayer structure including three or fewer layers. When the electron transport region forms a three-layer multilayer structure, the layers are defined as a first electron transport layer, a second electron transport layer and a third electron transport layer, in order from the positive electrode side. Preferably, these layers each independently have the function(s) of one or more selected from the group consisting of a hole-blocking layer, an electron transport layer and an electron injection layer.

The organic electroluminescent element, furthermore, may include a charge-generating layer between the positive electrode and the negative electrode. The organic electroluminescent element may include an additional light-emitting layer and an additional electron transport region, for example, as another unit.

The structure of the organic electroluminescent element according to an aspect of the present disclosure is not particularly limited. Examples, however, include structures 1) to 6) presented below.
1): Positive electrode/light-emitting layer/electron transport region/negative electrode
2): Positive electrode/hole transport layer/light-emitting layer/electron transport layer/negative electrode
3): Positive electrode/hole injection layer/hole transport layer/light-emitting layer/electron transport layer/electron injection layer/negative electrode
4): Positive electrode/hole injection layer/hole-generating layer/hole transport layer/light-emitting layer/electron transport layer/negative electrode
5): Positive electrode/hole injection layer/hole transport layer/electron-blocking layer/light-emitting layer/hole-blocking layer/electron transport layer/electron injection layer/negative electrode
6): Positive electrode/hole transport region/light-emitting layer/electron transport region/charge-generating layer/hole transport region/light-emitting layer/electron transport region/negative electrode

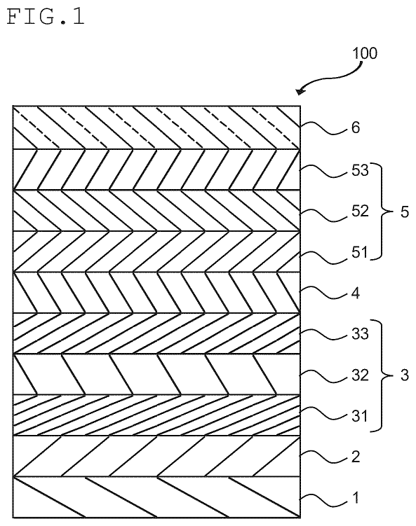

In the following, an organic electroluminescent element according to an aspect of the present disclosure will be described in further detail with reference to FIG. 1. FIG. 1 is a schematic cross-sectional view illustrating an example of a layer configuration of an organic electroluminescent element according to an aspect of the present disclosure.

An organic electroluminescence element according to an aspect of the present disclosure may be in a bottom-emission element configuration or a top-emission element configuration or may be in another known element configuration.

The organic electroluminescent element 100 includes a substrate 1, a positive electrode 2, a hole transport region 3, a light-emitting layer 4, an electron transport region 5 and a negative electrode 6 in this order. The hole transport region 3 may form a multilayer structure of a first hole transport layer 31, a second hole transport layer 32 and a third hole transport layer 33 in order from the positive electrode side. The electron transport region 5 may form a multilayer structure of a first electron transport layer 51, a second electron transport layer 52 and a third electron transport layer 53 in order from the positive electrode 2 side.

A subset of layers and/or regions in these layers and/or regions, however, may be omitted, or, conversely, other layers may be added. For example, a charge-generating layer may be provided between the electron transport region 5 and the negative electrode 6, or the hole transport region 3 may be omitted, with the light-emitting layer 4 directly provided on the positive electrode 2. The element, furthermore, may be in a configuration in which it includes a single layer combining functions of multiple layers, like an electron injection and transport layer, which combines the function of an electron injection layer and the function of an electron transport layer in a single layer, instead of the multiple layers. In addition, a single-layer hole transport layer 5 and a single-layer electron transport layer 7, for example, may each be composed of multiple layers.

### [Substrate 1]

There is no specific limitation on the substrate, and examples include a glass plate, a quartz plate, a plastic plate and a plastic film. In the case of a configuration in which the emitted light is extracted from the substrate 1 side, furthermore, the substrate 1 is transparent to the wavelength of the light emitted from the light-emitting layer 4. Alternatively, in the case of a configuration in which the emitted light is extracted from the negative electrode 6 side, the foundation 1 may include a reflector plate that reflects the light emitted from the light-emitting layer 4 toward the negative electrode 6 side.

Examples of plastic films having optical transparency include films made of materials such as polyethylene terephthalate (PET), polyethylene naphthalate (PEN), polyether sulfone (PES), polyether imide, polyether ether ketone, polyphenylene sulfide, polyarylate, polyimide, polycarbonate (PC), cellulose triacetate (TAC) and cellulose acetate propionate (CAP).

### [Positive Electrode 2]

Between the substrate 1 and the hole transport region 3, a positive electrode 2 is provided. The positive electrode 2 can serve a function as a positive electrode in passing an electric current through the organic electroluminescent element that is an aspect of the present disclosure.

In the case of a configuration in which the emitted light is extracted from the substrate 1 side, the positive electrode 2 is formed from a material that is permeable or substantially permeable to the emitted light or to a thickness that allows the emitted light to pass through in ten minutes. Alternatively, in the case of a configuration in which the issuance is extracted from the negative electrode 6 side, the positive electrode 2 may have the function of reflecting the light emitted from the light-emitting layer 4 toward the negative electrode 6 side. Specifically, the positive electrode 2 may be formed from a material that can reflect the emission wavelength from the light-emitting layer 4 sufficiently, or a multilayer structure of a material with high reflectance and an appropriate material with high transmittance may be formed.

In the case of a configuration in which the emitted light is extracted from the foundation 1 side, materials for use in the positive electrode 2 are not particularly limited. Examples, however, include indium tin oxide (ITO), indium zinc oxide (IZO), tin oxide, aluminum-doped tin oxide, magnesium indium oxide, nickel tungsten oxide and other metal oxides, metal nitrides, such as gallium nitride, metal selenides, such as zinc selenide, and metal sulfides, such as zinc sulfide.

It should be noted that for an organic electroluminescent element in a configuration in which the light is extracted solely from the negative electrode 6 side, the transmission characteristics of the positive electrode are not important. An example of a material for use in the positive electrode in that case, therefore, is gold, silver, iridium, molybdenum, palladium, platinum or an alloy of them.

### [Hole Transport Region 3]

Between the positive electrode 2 and the later-described light-emitting layer 4, a hole transport region 3 is provided. It may form a multilayer structure in which a first hole transport layer 31, a second hole transport layer 32 and a third hole transport layer 33 are provided in this order from the positive electrode 2 side.

The first hole transport layer 31, second hole transport layer 32 and third hole transport layer 33 forming the hole transport region 3 each independently have the function(s) of one or more selected from the group consisting of a hole injection layer, a hole-generating layer, a hole transport layer and an electron-blocking layer. For the functions of these layers, the details will be described below.

A hole injection layer has the function of efficiently injecting holes from the positive electrode 2 and also takes on the function of efficiently injecting holes into an adjacent layer.

Specific examples of materials for a hole injection layer include materials such as triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline copolymers, oligomers of electrically conductive polymers (thiophene oligomers in particular), porphyrin compounds, aromatic tertiary amine compounds and styrylamine compounds. Of these, porphyrin compounds, aromatic tertiary amine compounds and styrylamine compounds are particularly preferred, and aromatic tertiary amine compounds are preferred in particular. In the hole injection layer, furthermore, the later-described radialene compound (3) may be contained. In addition, the material may be a composition containing a material as listed above and the later-described radialene compound (3).

A hole-generating layer takes on the function of acquiring electrons from an occupied orbital of a compound forming an adjacent layer through the application of an electric field, thereby generating holes inside the adjacent layer (synonymous with injecting holes into the adjacent layer), and transporting the acquired electrons toward the positive electrode 2 side.

Specific examples of materials for a hole-generating layer include dipyrazino[2,3-f:2′,3′-h]quinoxaline-2,3,6,7,10,11-hexacarbonitrile (HAT-CN), 7,7,8,8-tetracyanoquinodimethane (TCNQ), 2,3,5,6-tetrafluoro-7,7,8,8-tetracyanoquinodimethane (F4-TCNQ), 11,11,12,12-tetracyano-2,6-naphthoquinodimethane (TNAP) and 15,15,16,16-tetracyanoanthraquinodimethane (TCAQ). In the hole-generating layer, furthermore, the later-described radialene compound (3) may be contained. In addition, the material may be a composition containing a material as listed above and the later-described radialene compound (3).

A hole transport layer takes on the function of transporting holes injected from the positive electrode 2, a hole injection layer or a hole-generating layer and injecting holes into an adjacent layer.

An electron-blocking layer takes on the function of blocking the injection of electrons into itself from an adjacent layer. By blocking electron injection, particularly from the light-emitting layer, it allows for an increased total number of electric charges contributing to light emission within the light-emitting layer and, in turn, contributes to enhancing the emission efficiency of the organic electroluminescent element.

Specific examples of materials for a hole transport layer and an electron-blocking layer include materials such as triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline copolymers, oligomers of electrically conductive polymers (thiophene oligomers in particular), porphyrin compounds, aromatic tertiary amine compounds and styrylamine compounds. Of these, porphyrin compounds, aromatic tertiary amine compounds and styrylamine compounds are particularly preferred, and aromatic tertiary amine compounds are preferred in particular. In the hole transport layer or electron transport layer, furthermore, the later-described radialene compound (3) may be contained.

The first hole transport layer 31 preferably takes on the function(s) of a hole injection layer and/or a hole-generating layer, more preferably the function of a hole injection layer.

The second hole transport layer 32 preferably takes on the function(s) of a hole-generating layer, a hole transport layer and/or an electron-blocking layer, more preferably the function of a hole transport layer.

The third hole transport layer 33 preferably takes on the function(s) of a hole transport layer and/or an electron-blocking layer, more preferably the function of an electron-blocking layer.

Specific examples of aromatic tertiary amine compounds and styrylamine compounds for use in a hole injection layer, a hole transport layer and an electron-blocking layer include compounds such as N,N,N′,N′-tetraphenyl-4,4′-diaminophenyl, N,N′-diphenyl-N,N′-bis(3-methylphenyl)-[1,1′-biphenyl]-4,4′-diamine (TPD), 2,2-bis(4-di-p-tolylaminophenyl)propane, 1,1-bis(4-di-p-tolylaminophenyl)cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenyl ether, 4,4′-bis(diphenylamino)quadriphenyl, N,N,N-tri(p-tolyl)amine, 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostyrylbenzene, N-phenylcarbazole, 4,4′-bis[N-(1-naphthyl)-N-phenylamino]biphenyl (NPD), 4,4′,4″-tris[N-(3-methylphenyl)-N-phenylamino]triphenylamine (MTDATA) and the compounds below.

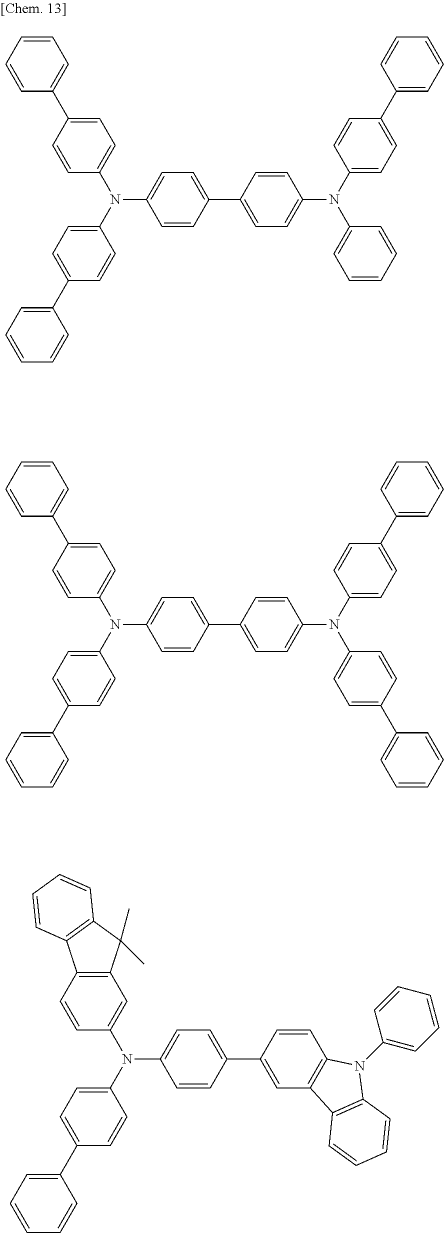 [SMILES: c1ccc(-c2ccc(N(c3ccccc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

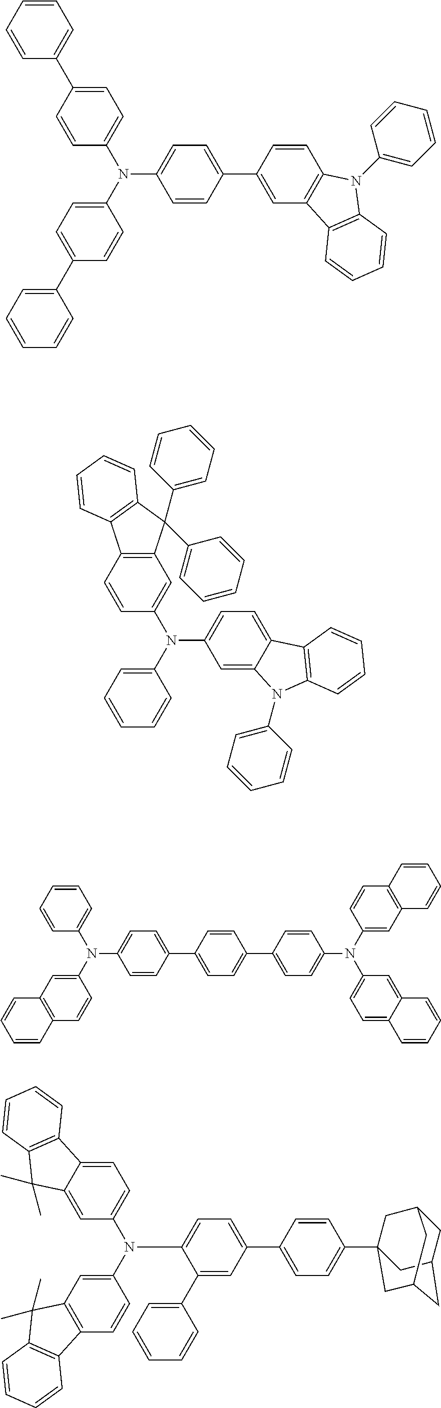

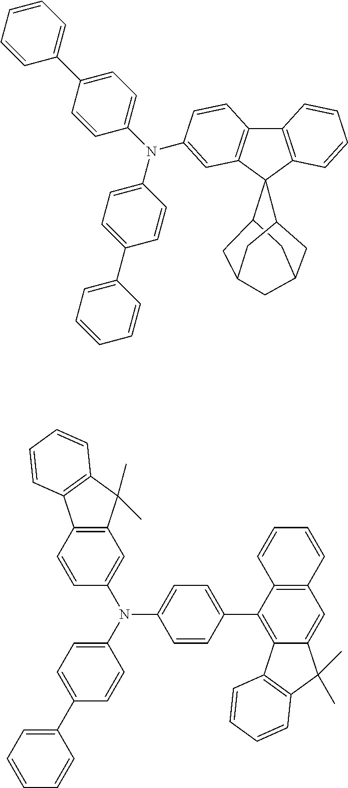 [SMILES: c1ccc(-c2ccc(N(c3ccc(-c4ccccc4)cc3)c3ccc4c(c3)C3(c5ccccc5-4)C4CC5CC(C4)CC3C5)cc2)cc1 ; CC1(C)c2ccccc2-c2ccc(N(c3ccc(-c4ccccc4)cc3)c3ccc(-c4c5c(cc6ccccc46)C(C)(C)c4ccccc4-5)cc3)cc21]

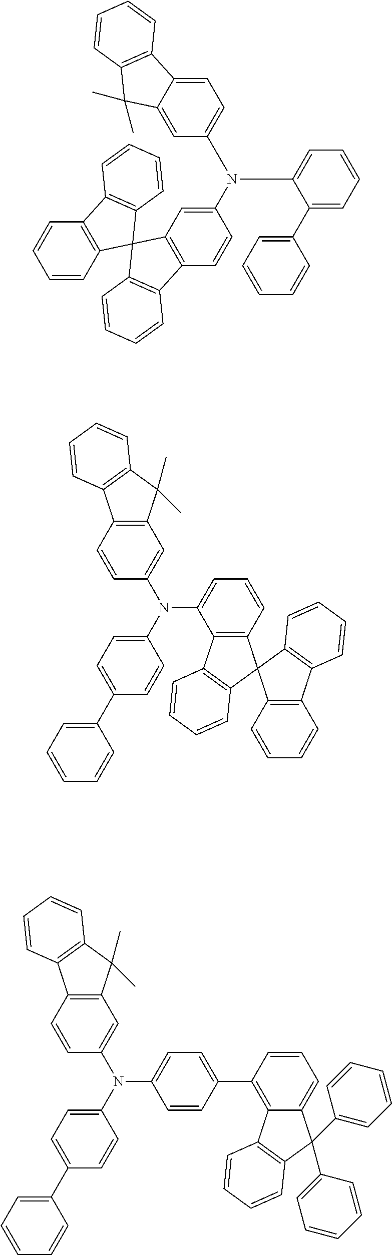 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; CC1(C)c2ccccc2-c2ccc(N(c3ccc(-c4ccccc4)cc3)c3cccc4c3-c3ccccc3C43c4ccccc4-c4ccccc43)cc21 ; CC1(C)c2ccccc2-c2ccc(N(c3ccc(-c4ccccc4)cc3)c3ccc(-c4cccc5c4-c4ccccc4C5(c4ccccc4)c4ccccc4)cc3)cc21]

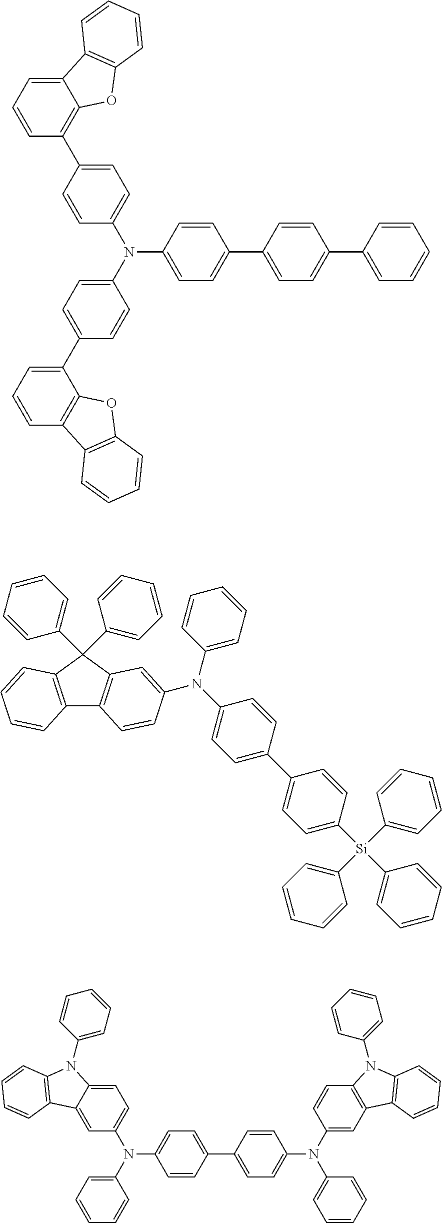 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

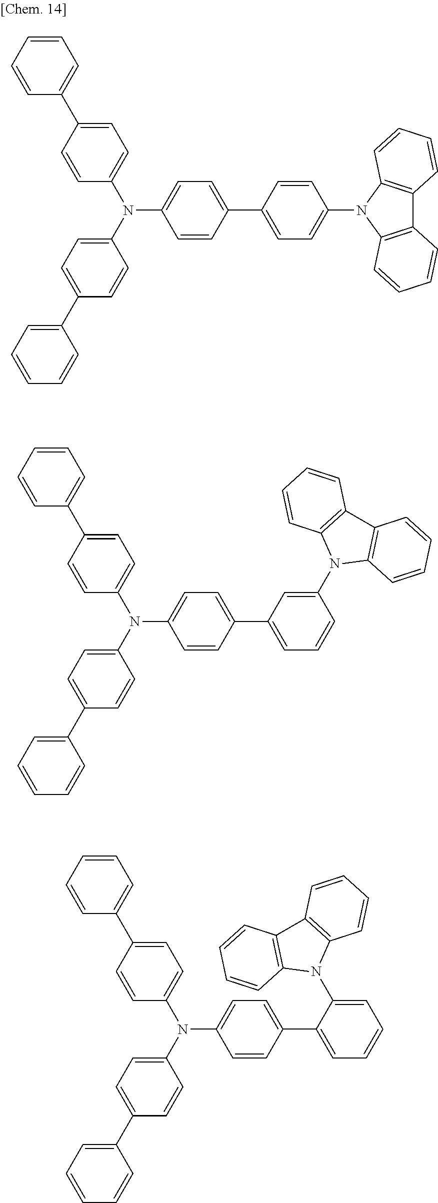 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4ccccc4)cc3)c3ccc(-c4ccccc4-n4c5ccccc5c5ccccc54)cc3)cc2)cc1]

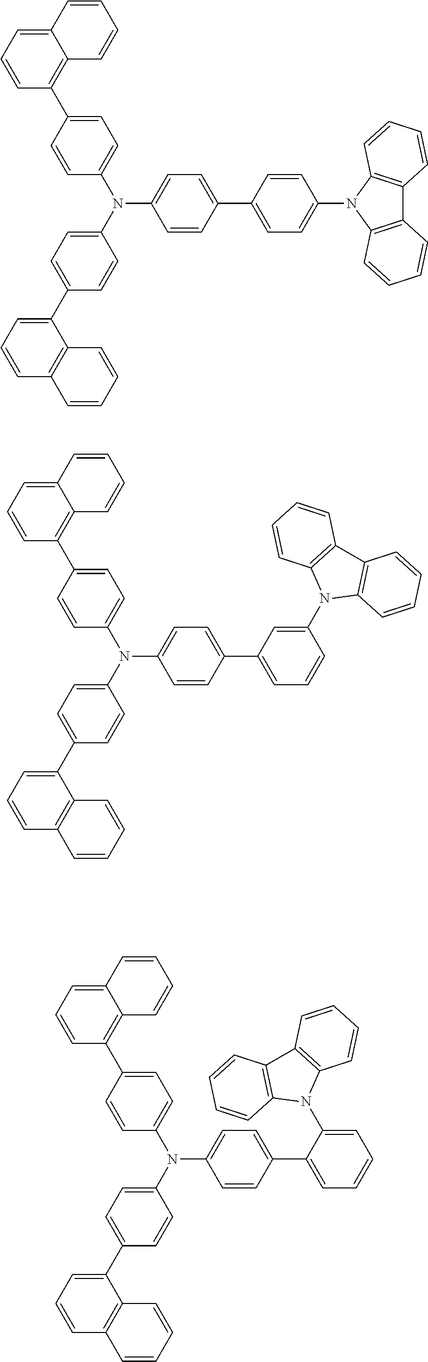 [SMILES: c1ccc2c(-c3ccc(N(c4ccc(-c5ccc(-n6c7ccccc7c7ccccc76)cc5)cc4)c4ccc(-c5cccc6ccccc56)cc4)cc3)cccc2c1 ; c1cc(-c2ccc(N(c3ccc(-c4cccc5ccccc45)cc3)c3ccc(-c4cccc5ccccc45)cc3)cc2)cc(-n2c3ccccc3c3ccccc32)c1 ; c1ccc(-n2c3ccccc3c3ccccc32)c(-c2ccc(N(c3ccc(-c4cccc5ccccc45)cc3)c3ccc(-c4cccc5ccccc45)cc3)cc2)c1]

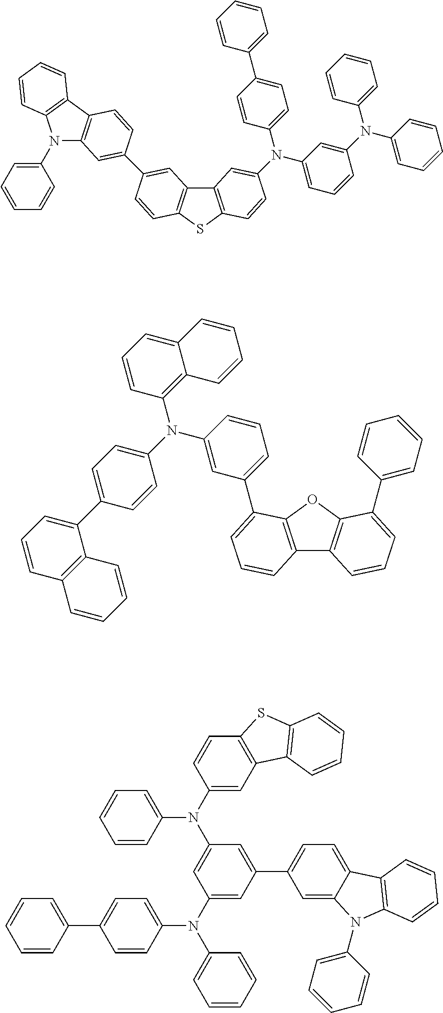 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(-c2cccc3c2oc2c(-c4cccc(N(c5ccc(-c6cccc7ccccc67)cc5)c5cccc6ccccc56)c4)cccc23)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1]

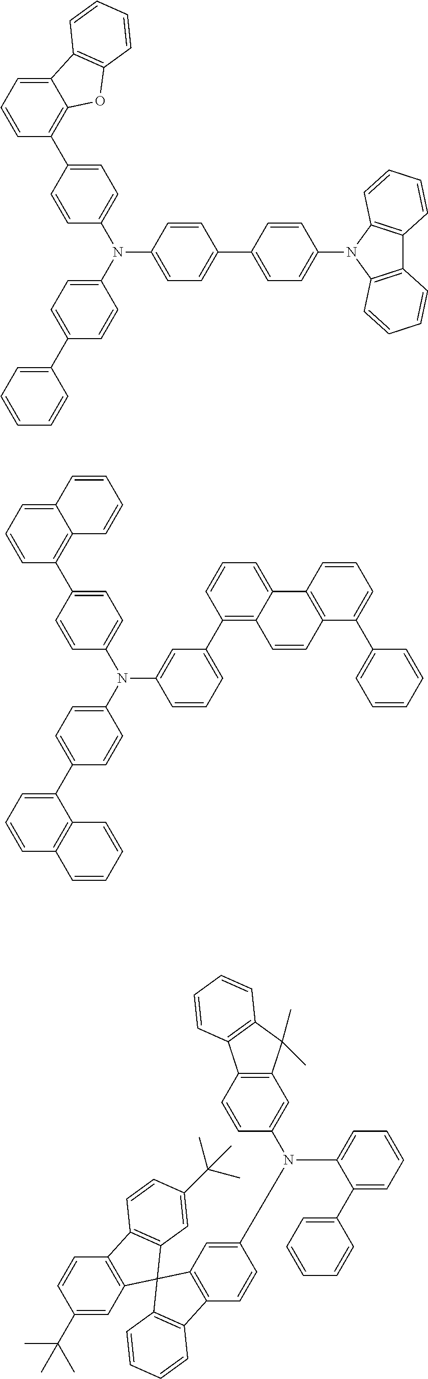 [SMILES: c1ccc(-c2ccc(N(c3ccc(-c4ccc(-n5c6ccccc6c6ccccc65)cc4)cc3)c3ccc(-c4cccc5c4oc4ccccc45)cc3)cc2)cc1 ; c1ccc(-c2cccc3c2ccc2c(-c4cccc(N(c5ccc(-c6cccc7ccccc67)cc5)c5ccc(-c6cccc7ccccc67)cc5)c4)cccc23)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

### <Radialene Compound>

The radialene compound contained in the hole transport region according to an aspect of the present disclosure is indicated by formula (3):

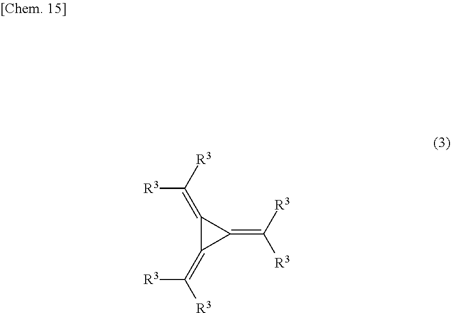

In formula (3),
R³ represents a cyano group, a halogen atom, a fluoroalkyl group, an acyl group, a sulfonyl group, a phosphoryl group, a substituted or unsubstituted aromatic hydrocarbon group or a substituted or unsubstituted aromatic heterocyclic group, independently at each occurrence.

A radialene compound indicated by formula (3) may be hereinafter referred to as a radialene compound (3). Preferred forms of the radialene compound (3), the definitions of substituents and their preferred specific examples are as follows.

For a halogen atom represented by R³, examples include a fluorine atom, a chlorine atom, a bromine atom and an iodine atom. Of these, a fluorine atom and a chlorine atom are preferred, and a fluorine atom is more preferred.

An acyl group represented by R³ is preferably a C2 to C10 acyl group including the carbon in the carbonyl moiety, more preferably a C2 to C10 acyl group substituted with a fluorine atom or atoms, even more preferably a C2 to C10 acyl group in which all hydrogen atoms have been replaced with fluorine atoms, particularly preferably a C2 to C6 acyl group in which all hydrogen atoms have been replaced with fluorine atoms.

A fluoroalkyl group represented by R³ is a C1 to C20 alkyl group in which one or more hydrogen atoms in the alkyl group have been replaced with fluorine atoms, preferably a C1 to C20 fluoroalkyl group in which all hydrogen atoms have been replaced with fluorine atoms, more preferably a C1 to C10 fluoroalkyl group, even more preferably a C1 to C6 fluoroalkyl group, particularly preferably a C1 to C3 fluoroalkyl group.

A substituted or unsubstituted aromatic hydrocarbon group represented by R³ is preferably an aromatic hydrocarbon group that may be substituted with one or more cyano groups, halogen atoms, acyl groups, sulfonyl groups or phosphoryl groups, in which the number of ring-constituting atoms is from 6 to 20 and that may be linked or annulated, more preferably an aromatic hydrocarbon group that may be substituted with cyano group(s) and/or halogen atom(s), in which the number of ring-constituting atoms is from 6 to 20 and that may be linked or annulated, particularly preferably a phenyl group or biphenylyl group substituted with cyano group(s) and/or fluorine atom(s).

A substituted or unsubstituted aromatic heterocyclic group represented by R³ is preferably an aromatic heterocyclic group that may be substituted with one or more cyano groups, halogen atoms, acyl groups, sulfonyl groups or phosphoryl groups, in which the number of ring-constituting atoms is from 6 to 20 and that may be linked or annulated, more preferably an aromatic heterocyclic group that may be substituted with cyano group(s) and/or halogen atom(s), in which the number of ring-constituting atoms is from 6 to 20 and that may be linked or annulated, particularly preferably a pyridyl group, pyrimidyl group, pyrazyl group or triazinyl group substituted with cyano group(s) and/or fluorine atom(s).

[Formula (3-a)]

The radialene compound (3) is preferably in a structure represented by formula (3-a).

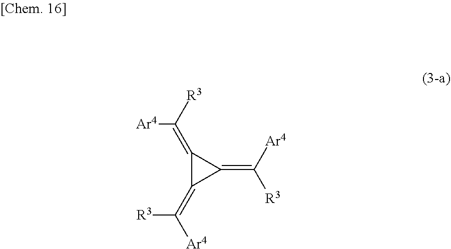

In formula (3-a),
Ar⁴ represents a substituted or unsubstituted aromatic hydrocarbon group or a substituted or unsubstituted aromatic heterocyclic group, independently at each occurrence.

Each substituent that a substituted or unsubstituted aromatic hydrocarbon group or substituted or unsubstituted aromatic heterocyclic group represented by R³ and Ar⁴ has is independently selected from
deuterium, a cyano group, a halogen atom, a fluoroalkyl group, an acyl group, a sulfonyl group and a phosphoryl group.

Preferred forms of R³ are the same as in formula (3).

A substituted or unsubstituted aromatic hydrocarbon group represented by Ar⁴ is preferably an aromatic hydrocarbon group that may be substituted with one or more cyano groups, halogen atoms, acyl groups, sulfonyl groups or phosphoryl groups, in which the number of ring-constituting atoms is from 6 to 20 and that may be linked or annulated, more preferably an aromatic hydrocarbon group that may be substituted with cyano group(s) and/or halogen atom(s), in which the number of ring-constituting atoms is from 6 to 20 and that may be linked or annulated, particularly preferably a phenyl group or biphenylyl group substituted with cyano group(s) and/or fluorine atom(s).

A substituted or unsubstituted aromatic heterocyclic group represented by Ar⁴ is preferably an aromatic heterocyclic group that may be substituted with one or more cyano groups, halogen atoms, acyl groups, sulfonyl groups or phosphoryl groups, in which the number of ring-constituting atoms is from 6 to 20 and that may be linked or annulated, more preferably an aromatic heterocyclic group that may be substituted with cyano group(s) and/or halogen atom(s), in which the number of ring-constituting atoms is from 6 to 20 and that may be linked or annulated, particularly preferably a pyridyl group, pyrimidyl group, pyrazyl group or triazinyl group substituted with cyano group(s) and/or fluorine atom(s).

[Formula (3-b)]

Radialene compounds (3) and (3-a) are preferably in a structure represented by formula (3-b).

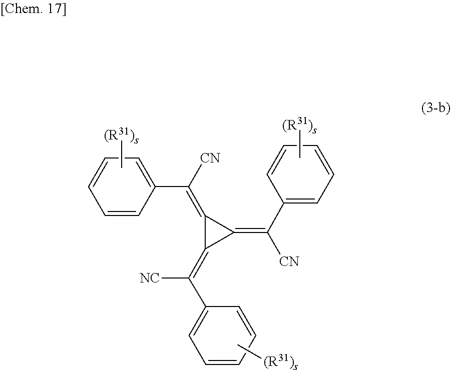

In formula (3-b),
R³¹ is selected from deuterium, a cyano group, a halogen atom, a fluoroalkyl group, an acyl group, a sulfonyl group and a phosphoryl group, independently at each occurrence.
s is an integer of 1 to 5, independently at each occurrence.

Preferred forms of a halogen atom, a fluoroalkyl group and an acyl group represented by R³¹ are the same as for R³.

R³¹ is preferably selected from a cyano group, a fluorine atom and a fluoroalkyl group. More preferably, the R³¹s are cyano group(s) and/or fluorine atom(s), even more preferably cyano group(s) and fluorine atom(s).

[Preferred Examples of Radialene Compounds (3), (3-a) and (3-b)]

The radialene compound (3) that is an aspect of the present disclosure is more preferably a radialene compound represented by any one of compounds 3-1 to 3-148 below.

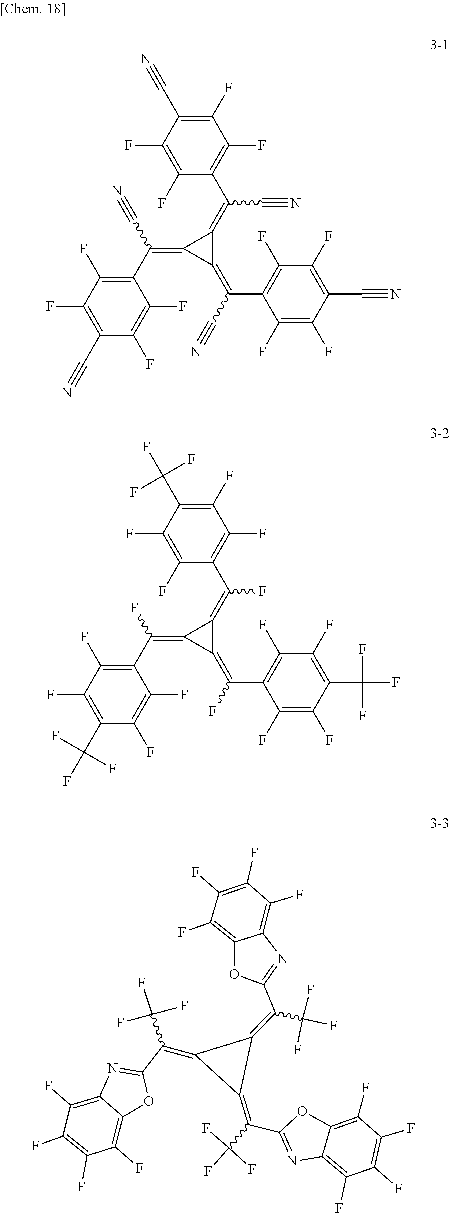 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; FC(=C1C(=C(F)c2c(F)c(F)c(C(F)(F)F)c(F)c2F)C1=C(F)c1c(F)c(F)c(C(F)(F)F)c(F)c1F)c1c(F)c(F)c(C(F)(F)F)c(F)c1F ; Fc1c(F)c(F)c2oc(C(=C3C(=C(c4nc5c(F)c(F)c(F)c(F)c5o4)C(F)(F)F)C3=C(c3nc4c(F)c(F)c(F)c(F)c4o3)C(F)(F)F)C(F)(F)F)nc2c1F]

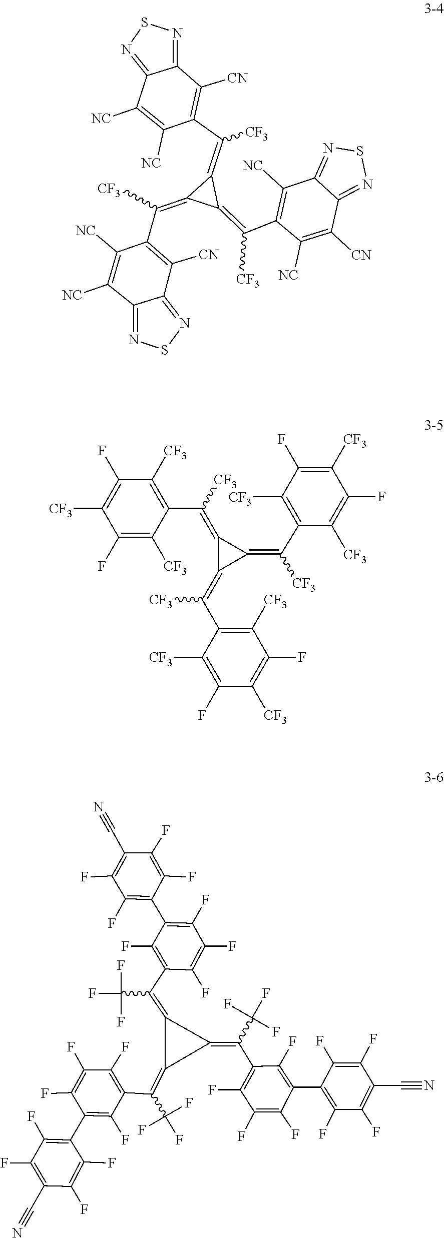 [SMILES: N#Cc1c(C(=C2C(=C(c3c(C#N)c(C#N)c4nsnc4c3C#N)C(F)(F)F)C2=C(c2c(C#N)c(C#N)c3nsnc3c2C#N)C(F)(F)F)C(F)(F)F)c(C#N)c2nsnc2c1C#N ; Fc1c(C(F)(F)F)c(F)c(C(F)(F)F)c(C(=C2C(=C(c3c(C(F)(F)F)c(F)c(C(F)(F)F)c(F)c3C(F)(F)F)C(F)(F)F)C2=C(c2c(C(F)(F)F)c(F)c(C(F)(F)F)c(F)c2C(F)(F)F)C(F)(F)F)C(F)(F)F)c1C(F)(F)F ; N#Cc1c(F)c(F)c(-c2c(F)c(F)c(F)c(C(=C3C(=C(c4c(F)c(F)c(F)c(-c5c(F)c(F)c(C#N)c(F)c5F)c4F)C(F)(F)F)C3=C(c3c(F)c(F)c(F)c(-c4c(F)c(F)c(C#N)c(F)c4F)c3F)C(F)(F)F)C(F)(F)F)c2F)c(F)c1F]

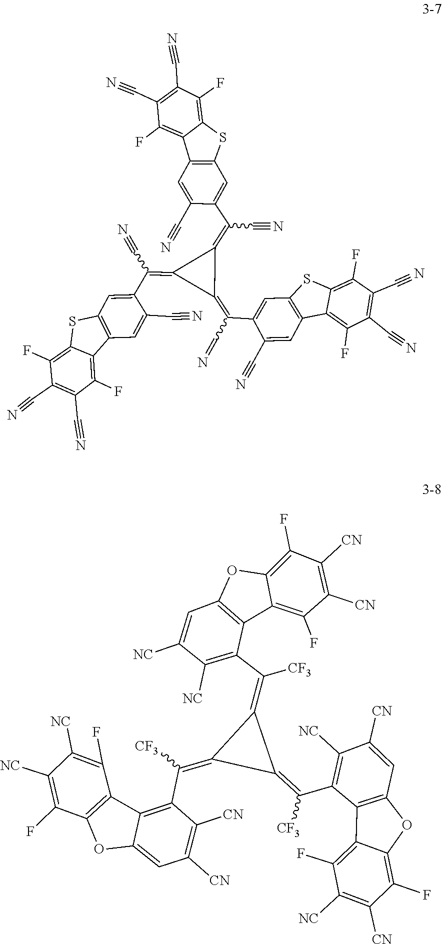

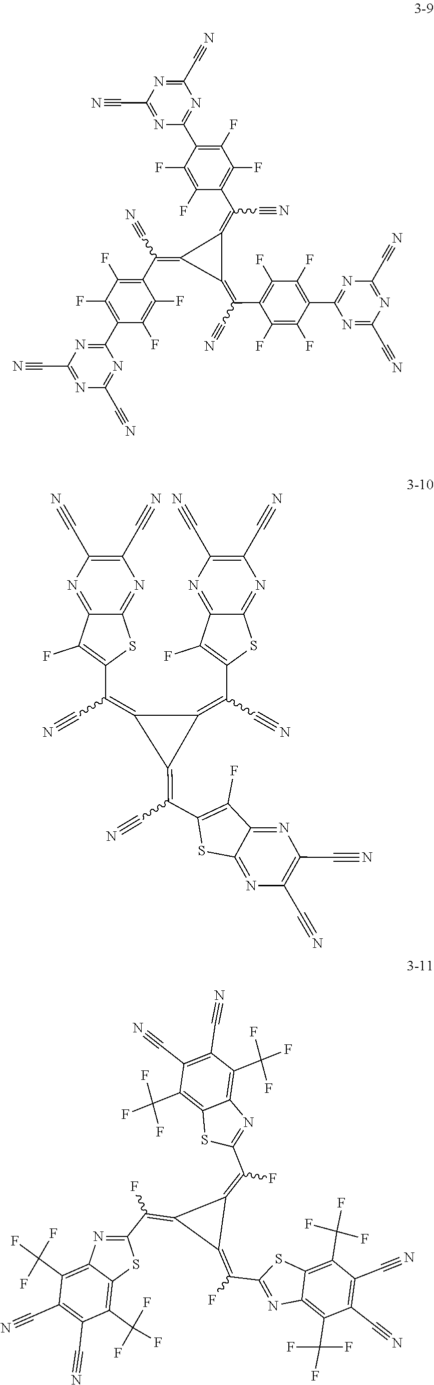 [SMILES: N#CC(=C1C(=C(C#N)c2c(F)c(F)c(-c3nc(C#N)nc(C#N)n3)c(F)c2F)C1=C(C#N)c1c(F)c(F)c(-c2nc(C#N)nc(C#N)n2)c(F)c1F)c1c(F)c(F)c(-c2nc(C#N)nc(C#N)n2)c(F)c1F ; N#CC(=C1C(=C(C#N)c2sc3nc(C#N)c(C#N)nc3c2F)C1=C(C#N)c1sc2nc(C#N)c(C#N)nc2c1F)c1sc2nc(C#N)c(C#N)nc2c1F ; N#Cc1c(C#N)c(C(F)(F)F)c2sc(C(F)=C3C(=C(F)c4nc5c(C(F)(F)F)c(C#N)c(C#N)c(C(F)(F)F)c5s4)C3=C(F)c3nc4c(C(F)(F)F)c(C#N)c(C#N)c(C(F)(F)F)c4s3)nc2c1C(F)(F)F]

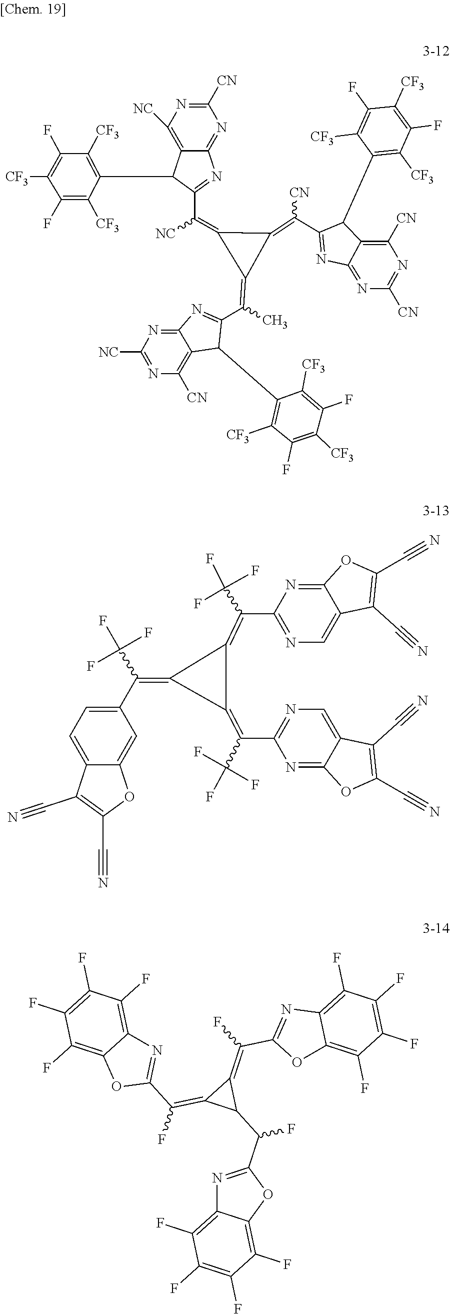

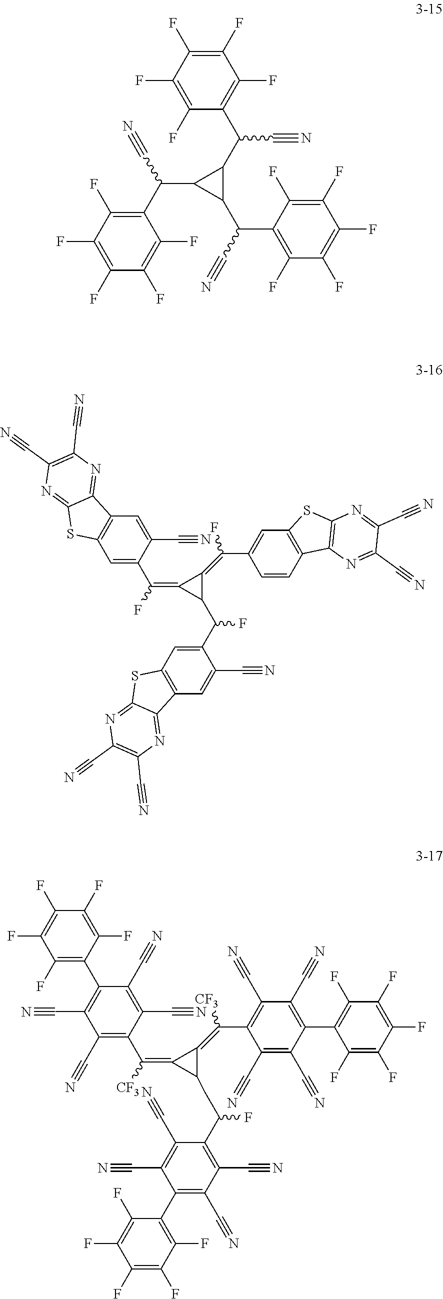 [SMILES: N#CC(c1c(F)c(F)c(F)c(F)c1F)C1C(C(C#N)c2c(F)c(F)c(F)c(F)c2F)C1C(C#N)c1c(F)c(F)c(F)c(F)c1F ; N#Cc1cc2c(cc1C(F)=C1C(=C(F)c3ccc4c(c3)sc3nc(C#N)c(C#N)nc34)C1C(F)c1cc3sc4nc(C#N)c(C#N)nc4c3cc1C#N)sc1nc(C#N)c(C#N)nc12 ; N#Cc1c(C#N)c(-c2c(F)c(F)c(F)c(F)c2F)c(C#N)c(C#N)c1C(=C1C(=C(c2c(C#N)c(C#N)c(-c3c(F)c(F)c(F)c(F)c3F)c(C#N)c2C#N)C(F)(F)F)C1CC(F)c1c(C#N)c(C#N)c(-c2c(F)c(F)c(F)c(F)c2F)c(C#N)c1C#N)C(F)(F)F]

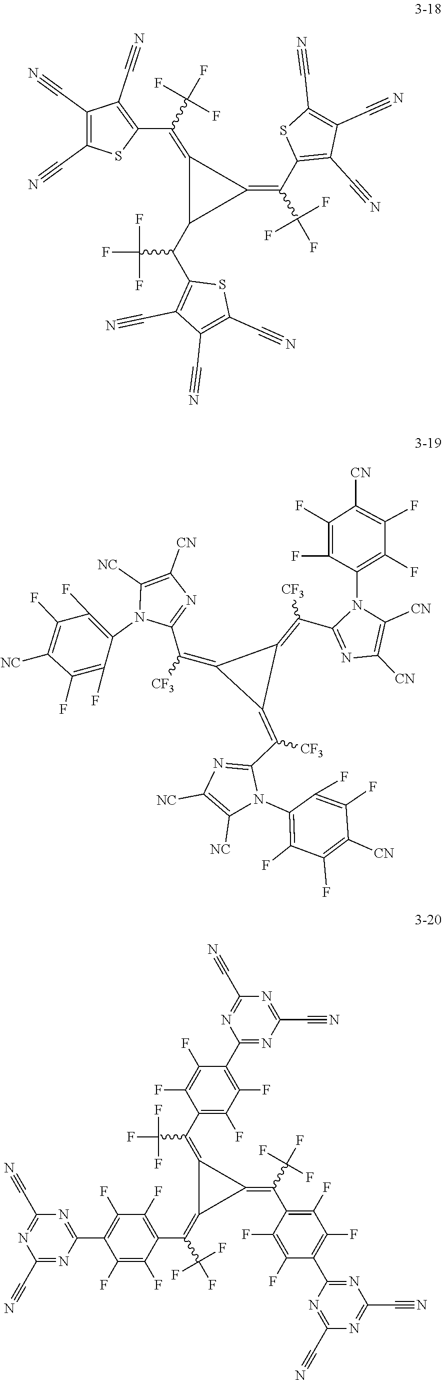 [SMILES: N#Cc1sc(C(=C2C(=C(c3sc(C#N)c(C#N)c3C#N)C(F)(F)F)C2C(c2sc(C#N)c(C#N)c2C#N)C(F)(F)F)C(F)(F)F)c(C#N)c1C#N ; N#Cc1nc(C(=C2C(=C(c3nc(C#N)c(C#N)n3-c3c(F)c(F)c(C#N)c(F)c3F)C(F)(F)F)C2=C(c2nc(C#N)c(C#N)n2-c2c(F)c(F)c(C#N)c(F)c2F)C(F)(F)F)C(F)(F)F)n(-c2c(F)c(F)c(C#N)c(F)c2F)c1C#N ; N#Cc1nc(C#N)nc(-c2c(F)c(F)c(C(=C3C(=C(c4c(F)c(F)c(-c5nc(C#N)nc(C#N)n5)c(F)c4F)C(F)(F)F)C3=C(c3c(F)c(F)c(-c4nc(C#N)nc(C#N)n4)c(F)c3F)C(F)(F)F)C(F)(F)F)c(F)c2F)n1]

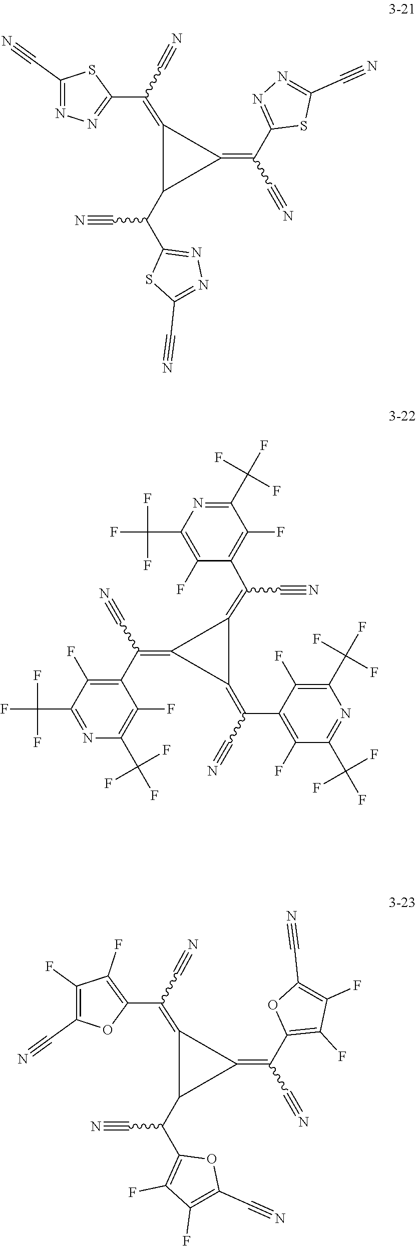 [SMILES: N#CC(=C1C(=C(C#N)c2nnc(C#N)s2)C1C(C#N)c1nnc(C#N)s1)c1nnc(C#N)s1 ; N#CC(=C1C(=C(C#N)c2c(F)c(C(F)(F)F)nc(C(F)(F)F)c2F)C1=C(C#N)c1c(F)c(C(F)(F)F)nc(C(F)(F)F)c1F)c1c(F)c(C(F)(F)F)nc(C(F)(F)F)c1F ; N#CC(=C1C(=C(C#N)c2oc(C#N)c(F)c2F)C1C(C#N)c1oc(C#N)c(F)c1F)c1oc(C#N)c(F)c1F]

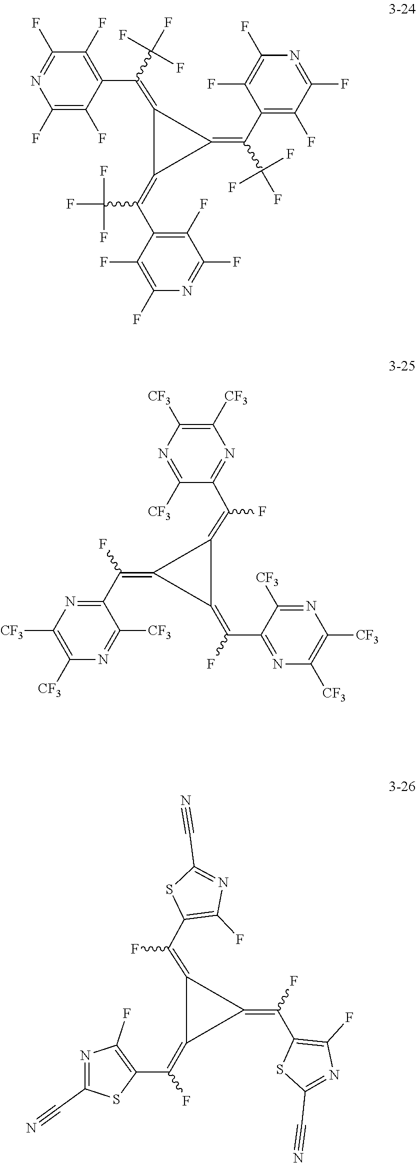 [SMILES: Fc1nc(F)c(F)c(C(=C2C(=C(c3c(F)c(F)nc(F)c3F)C(F)(F)F)C2=C(c2c(F)c(F)nc(F)c2F)C(F)(F)F)C(F)(F)F)c1F ; FC(=C1C(=C(F)c2nc(C(F)(F)F)c(C(F)(F)F)nc2C(F)(F)F)C1=C(F)c1nc(C(F)(F)F)c(C(F)(F)F)nc1C(F)(F)F)c1nc(C(F)(F)F)c(C(F)(F)F)nc1C(F)(F)F ; N#Cc1nc(F)c(C(F)=C2C(=C(F)c3sc(C#N)nc3F)C2=C(F)c2sc(C#N)nc2F)s1]

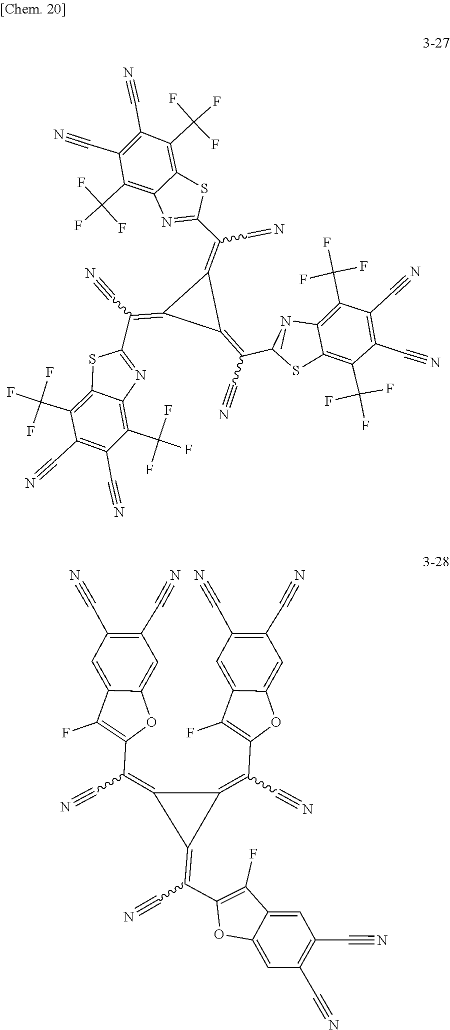

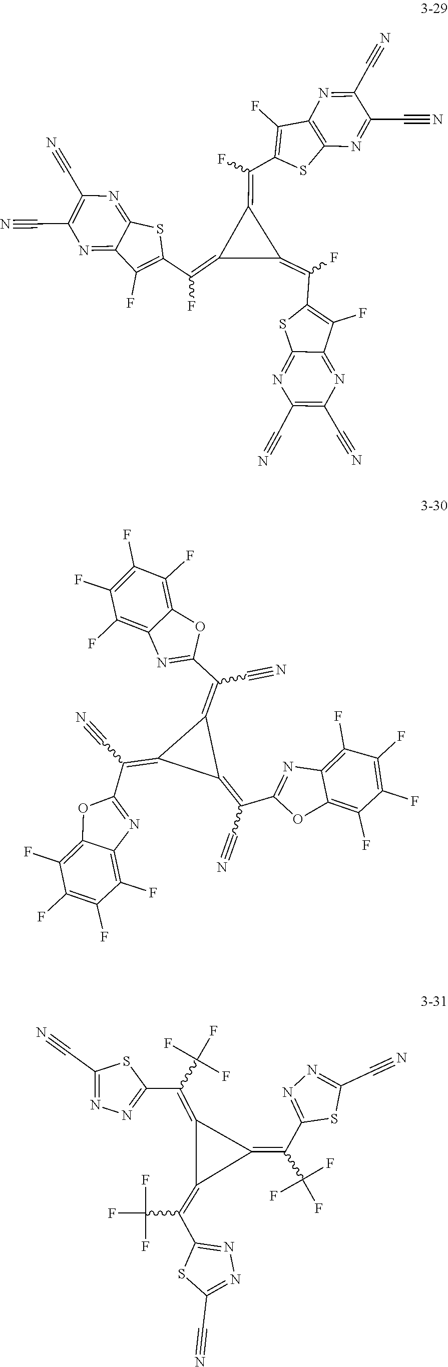 [SMILES: N#Cc1nc2sc(C(F)=C3C(=C(F)c4sc5nc(C#N)c(C#N)nc5c4F)C3=C(F)c3sc4nc(C#N)c(C#N)nc4c3F)c(F)c2nc1C#N ; N#CC(=C1C(=C(C#N)c2nc3c(F)c(F)c(F)c(F)c3o2)C1=C(C#N)c1nc2c(F)c(F)c(F)c(F)c2o1)c1nc2c(F)c(F)c(F)c(F)c2o1 ; N#Cc1nnc(C(=C2C(=C(c3nnc(C#N)s3)C(F)(F)F)C2=C(c2nnc(C#N)s2)C(F)(F)F)C(F)(F)F)s1]

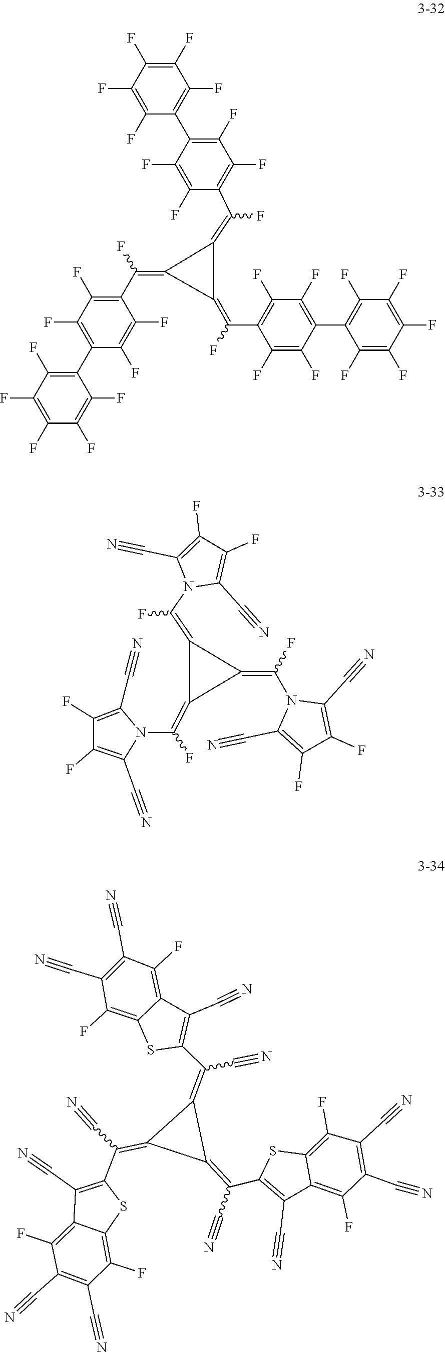 [SMILES: FC(=C1C(=C(F)c2c(F)c(F)c(-c3c(F)c(F)c(F)c(F)c3F)c(F)c2F)C1=C(F)c1c(F)c(F)c(-c2c(F)c(F)c(F)c(F)c2F)c(F)c1F)c1c(F)c(F)c(-c2c(F)c(F)c(F)c(F)c2F)c(F)c1F ; N#Cc1c(F)c(F)c(C#N)n1C(F)=C1C(=C(F)n2c(C#N)c(F)c(F)c2C#N)C1=C(F)n1c(C#N)c(F)c(F)c1C#N ; N#CC(=C1C(=C(C#N)c2sc3c(F)c(C#N)c(C#N)c(F)c3c2C#N)C1=C(C#N)c1sc2c(F)c(C#N)c(C#N)c(F)c2c1C#N)c1sc2c(F)c(C#N)c(C#N)c(F)c2c1C#N]

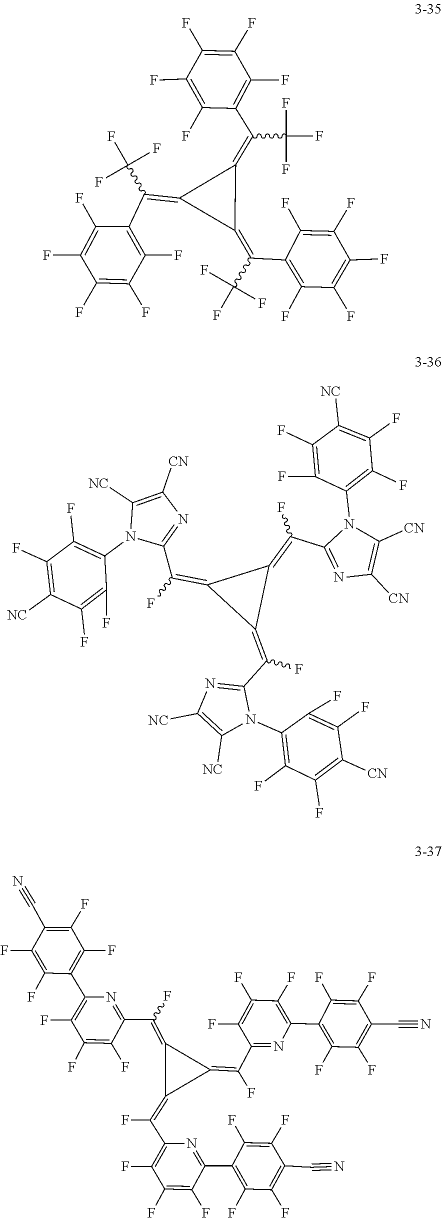 [SMILES: Fc1c(F)c(F)c(C(=C2C(=C(c3c(F)c(F)c(F)c(F)c3F)C(F)(F)F)C2=C(c2c(F)c(F)c(F)c(F)c2F)C(F)(F)F)C(F)(F)F)c(F)c1F ; N#Cc1nc(C(F)=C2C(=C(F)c3nc(C#N)c(C#N)n3-c3c(F)c(F)c(C#N)c(F)c3F)C2=C(F)c2nc(C#N)c(C#N)n2-c2c(F)c(F)c(C#N)c(F)c2F)n(-c2c(F)c(F)c(C#N)c(F)c2F)c1C#N ; N#Cc1c(F)c(F)c(-c2nc(C(F)=C3C(=C(F)c4nc(-c5c(F)c(F)c(C#N)c(F)c5F)c(F)c(F)c4F)C3=C(F)c3nc(-c4c(F)c(F)c(C#N)c(F)c4F)c(F)c(F)c3F)c(F)c(F)c2F)c(F)c1F]

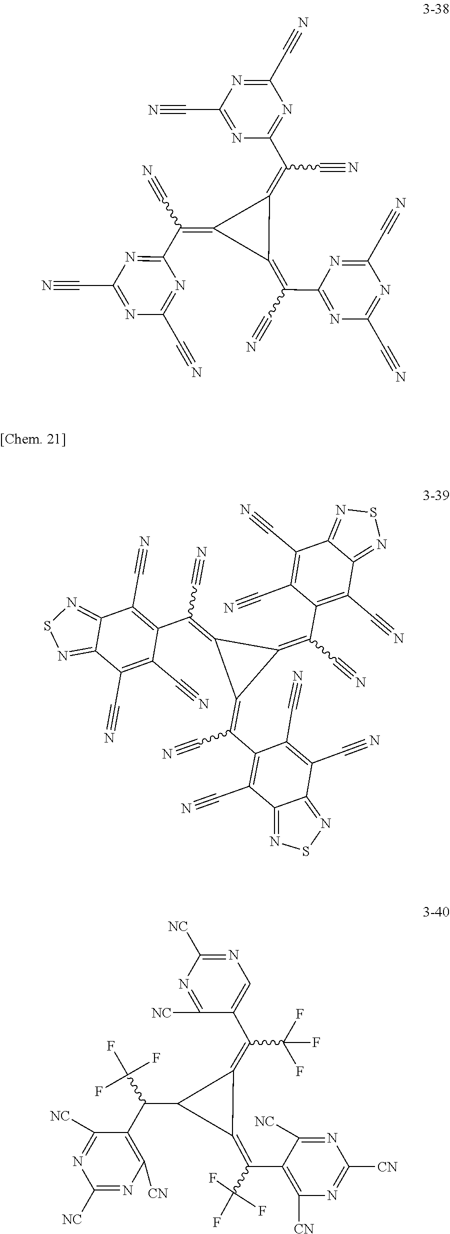 [SMILES: N#CC(=C1C(=C(C#N)c2nc(C#N)nc(C#N)n2)C1=C(C#N)c1nc(C#N)nc(C#N)n1)c1nc(C#N)nc(C#N)n1 ; N#CC(=C1C(=C(C#N)c2c(C#N)c(C#N)c3nsnc3c2C#N)C1=C(C#N)c1c(C#N)c(C#N)c2nsnc2c1C#N)c1c(C#N)c(C#N)c2nsnc2c1C#N ; N#Cc1ncc(C(=C2C(=C(c3c(C#N)nc(C#N)nc3C#N)C(F)(F)F)C2C(c2c(C#N)nc(C#N)nc2C#N)C(F)(F)F)C(F)(F)F)c(C#N)n1]

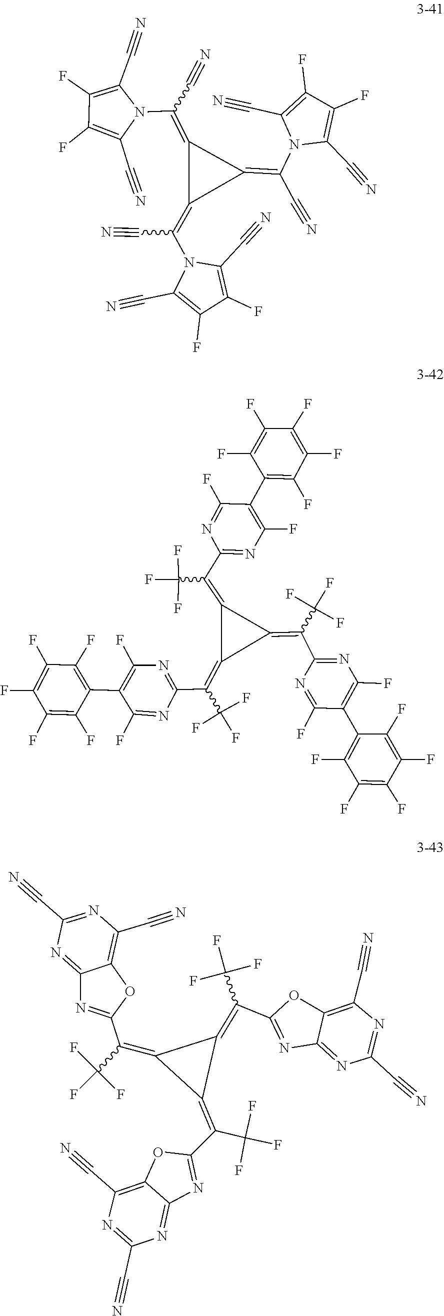 [SMILES: N#CC(=C1C(=C(C#N)n2c(C#N)c(F)c(F)c2C#N)C1=C(C#N)n1c(C#N)c(F)c(F)c1C#N)n1c(C#N)c(F)c(F)c1C#N ; Fc1nc(C(=C2C(=C(c3nc(F)c(-c4c(F)c(F)c(F)c(F)c4F)c(F)n3)C(F)(F)F)C2=C(c2nc(F)c(-c3c(F)c(F)c(F)c(F)c3F)c(F)n2)C(F)(F)F)C(F)(F)F)nc(F)c1-c1c(F)c(F)c(F)c(F)c1F ; N#Cc1nc(C#N)c2oc(C(=C3C(=C(c4nc5nc(C#N)nc(C#N)c5o4)C(F)(F)F)C3=C(c3nc4nc(C#N)nc(C#N)c4o3)C(F)(F)F)C(F)(F)F)nc2n1]

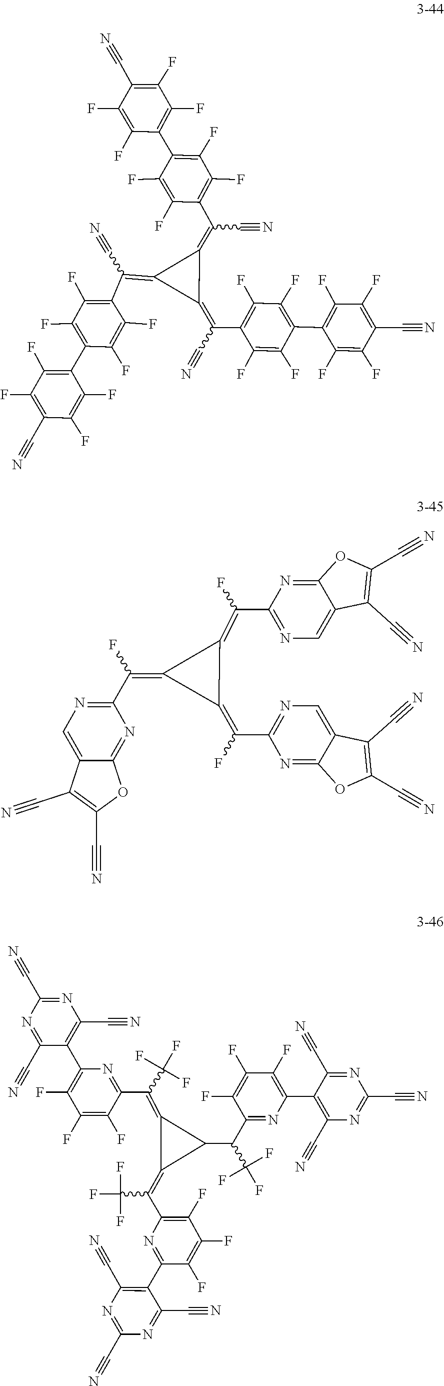 [SMILES: N#CC(=C1C(=C(C#N)c2c(F)c(F)c(-c3c(F)c(F)c(C#N)c(F)c3F)c(F)c2F)C1=C(C#N)c1c(F)c(F)c(-c2c(F)c(F)c(C#N)c(F)c2F)c(F)c1F)c1c(F)c(F)c(-c2c(F)c(F)c(C#N)c(F)c2F)c(F)c1F ; N#Cc1oc2nc(C(F)=C3C(=C(F)c4ncc5c(C#N)c(C#N)oc5n4)C3=C(F)c3ncc4c(C#N)c(C#N)oc4n3)ncc2c1C#N ; N#Cc1nc(C#N)c(-c2nc(C(=C3C(=C(c4nc(-c5c(C#N)nc(C#N)nc5C#N)c(F)c(F)c4F)C(F)(F)F)C3C(c3nc(-c4c(C#N)nc(C#N)nc4C#N)c(F)c(F)c3F)C(F)(F)F)C(F)(F)F)c(F)c(F)c2F)c(C#N)n1]

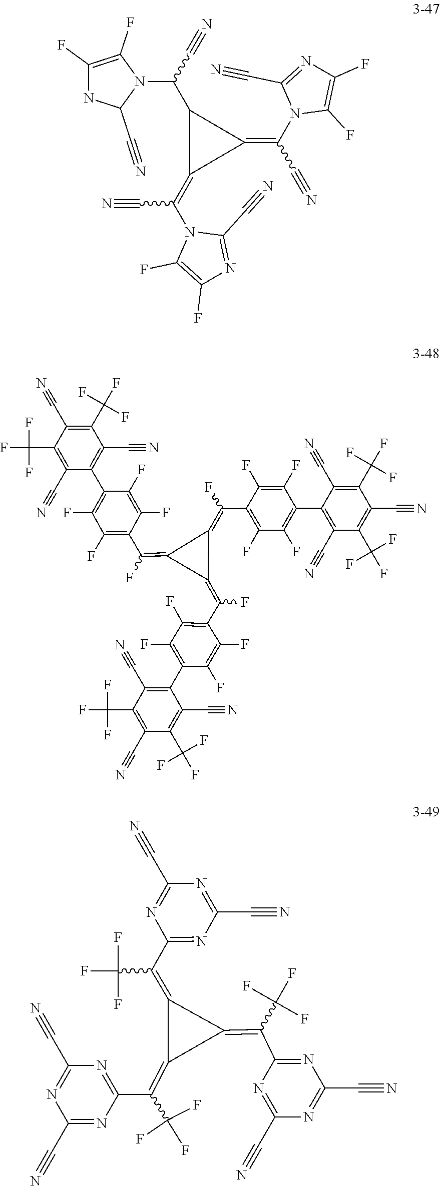 [SMILES: N#CC(=C1C(=C(C#N)n2c(C#N)nc(F)c2F)C1C(C#N)N1C(F)=C(F)NC1C#N)n1c(C#N)nc(F)c1F ; N#Cc1c(-c2c(F)c(F)c(C(F)=C3C(=C(F)c4c(F)c(F)c(-c5c(C#N)c(C(F)(F)F)c(C#N)c(C(F)(F)F)c5C#N)c(F)c4F)C3=C(F)c3c(F)c(F)c(-c4c(C#N)c(C(F)(F)F)c(C#N)c(C(F)(F)F)c4C#N)c(F)c3F)c(F)c2F)c(C#N)c(C(F)(F)F)c(C#N)c1C(F)(F)F ; N#Cc1nc(C#N)nc(C(=C2C(=C(c3nc(C#N)nc(C#N)n3)C(F)(F)F)C2=C(c2nc(C#N)nc(C#N)n2)C(F)(F)F)C(F)(F)F)n1]

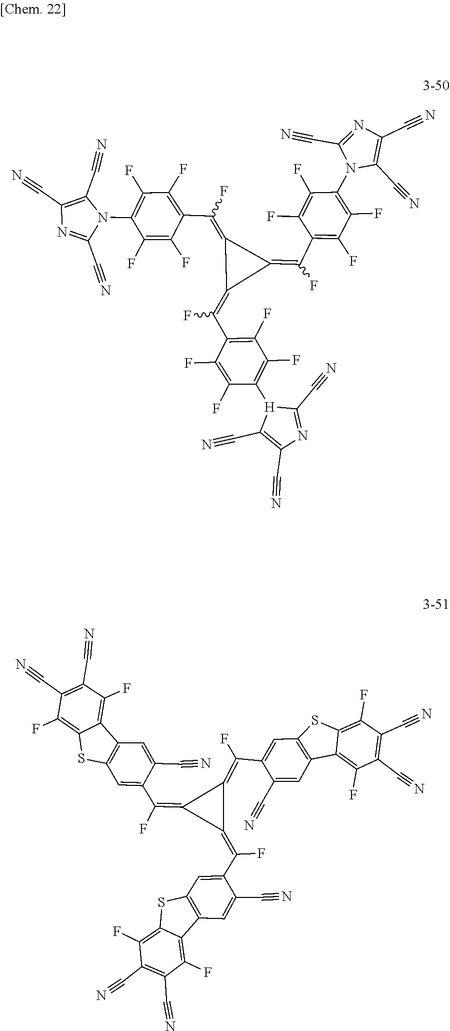

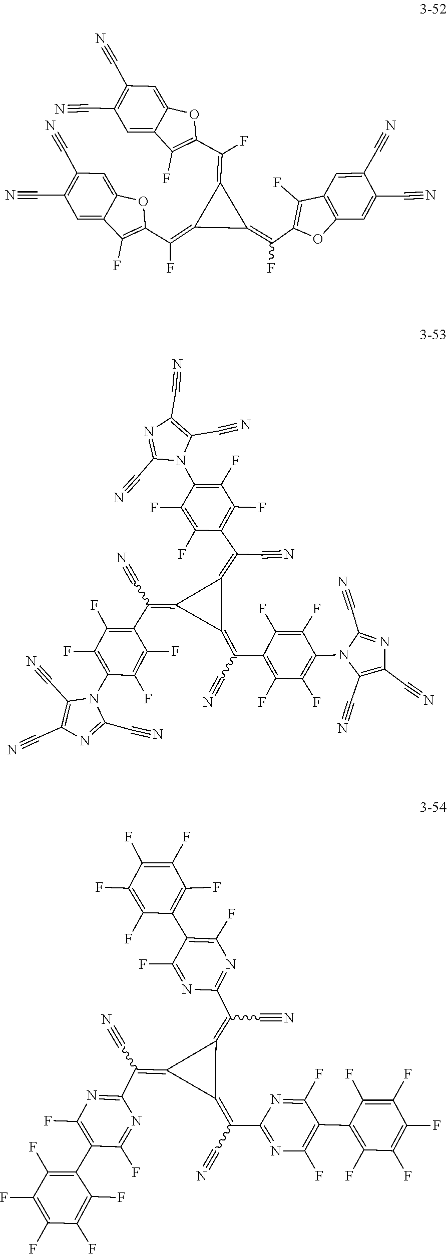 [SMILES: N#Cc1cc2oc(C(F)=C3C(=C(F)c4oc5cc(C#N)c(C#N)cc5c4F)C3=C(F)c3oc4cc(C#N)c(C#N)cc4c3F)c(F)c2cc1C#N ; N#CC(=C1C(=C(C#N)c2c(F)c(F)c(-n3c(C#N)nc(C#N)c3C#N)c(F)c2F)C1=C(C#N)c1c(F)c(F)c(-n2c(C#N)nc(C#N)c2C#N)c(F)c1F)c1c(F)c(F)c(-n2c(C#N)nc(C#N)c2C#N)c(F)c1F ; N#CC(=C1C(=C(C#N)c2nc(F)c(-c3c(F)c(F)c(F)c(F)c3F)c(F)n2)C1=C(C#N)c1nc(F)c(-c2c(F)c(F)c(F)c(F)c2F)c(F)n1)c1nc(F)c(-c2c(F)c(F)c(F)c(F)c2F)c(F)n1]

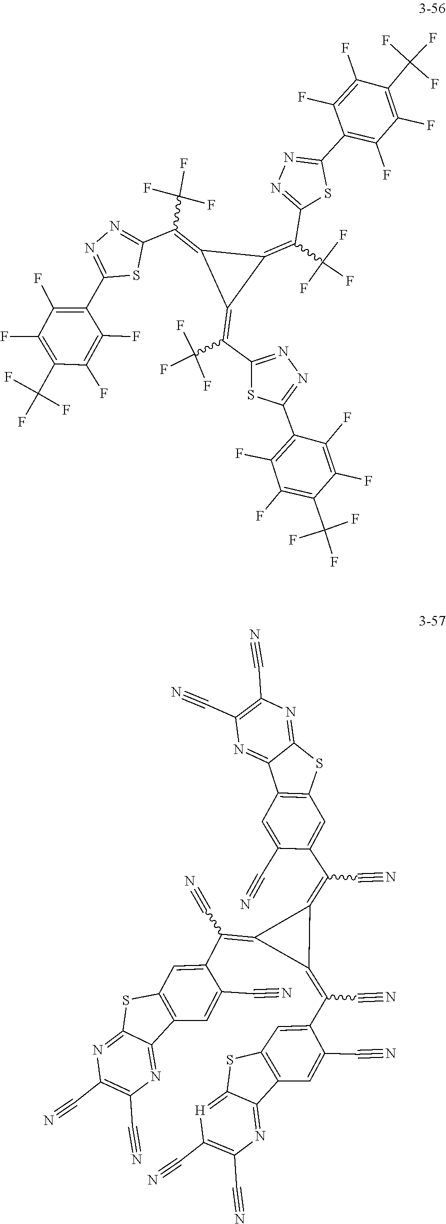

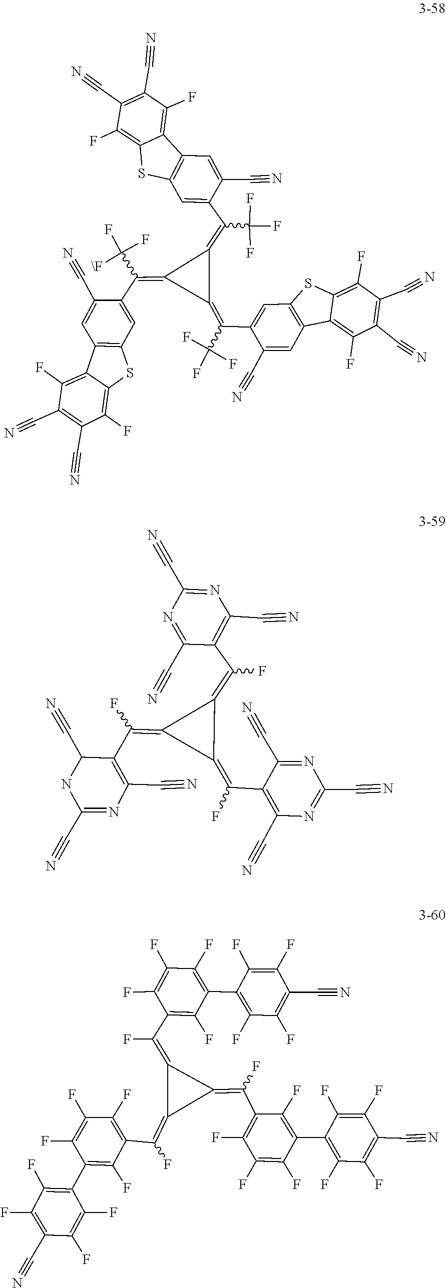

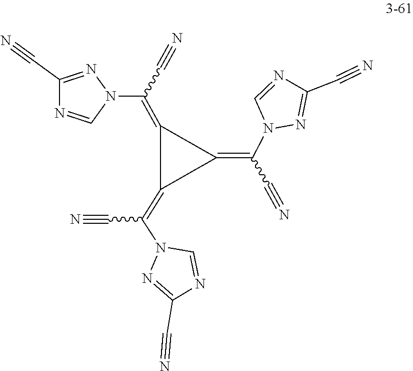 [SMILES: N#CC(=C1C(=C(C#N)n2cnc(C#N)n2)C1=C(C#N)n1cnc(C#N)n1)n1cnc(C#N)n1]

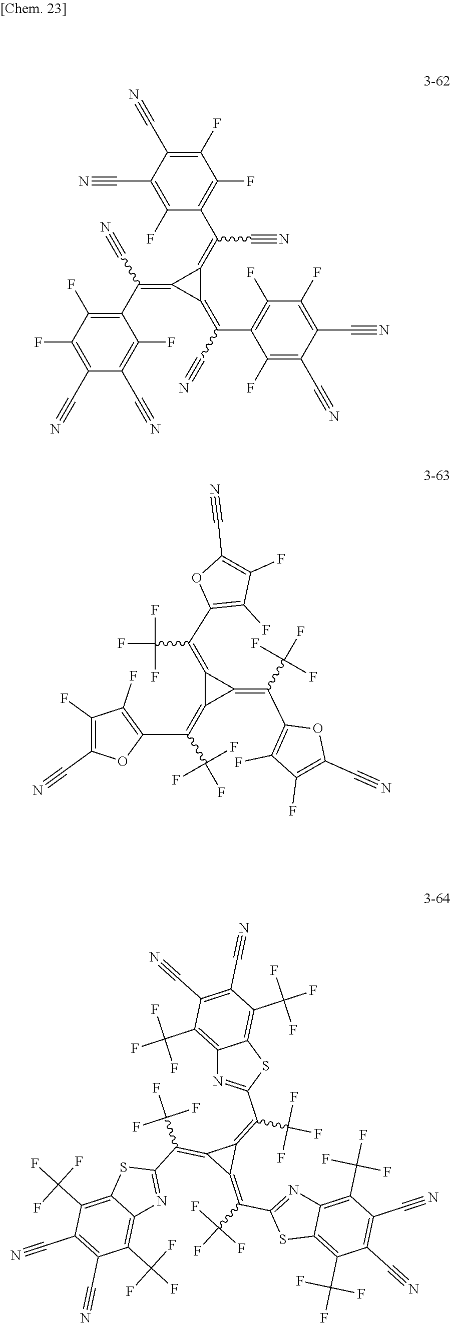 [SMILES: N#CC(=C1C(=C(C#N)c2c(F)c(F)c(C#N)c(C#N)c2F)C1=C(C#N)c1c(F)c(F)c(C#N)c(C#N)c1F)c1c(F)c(F)c(C#N)c(C#N)c1F ; N#Cc1oc(C(=C2C(=C(c3oc(C#N)c(F)c3F)C(F)(F)F)C2=C(c2oc(C#N)c(F)c2F)C(F)(F)F)C(F)(F)F)c(F)c1F ; N#Cc1c(C#N)c(C(F)(F)F)c2sc(C(=C3C(=C(c4nc5c(C(F)(F)F)c(C#N)c(C#N)c(C(F)(F)F)c5s4)C(F)(F)F)C3=C(c3nc4c(C(F)(F)F)c(C#N)c(C#N)c(C(F)(F)F)c4s3)C(F)(F)F)C(F)(F)F)nc2c1C(F)(F)F]

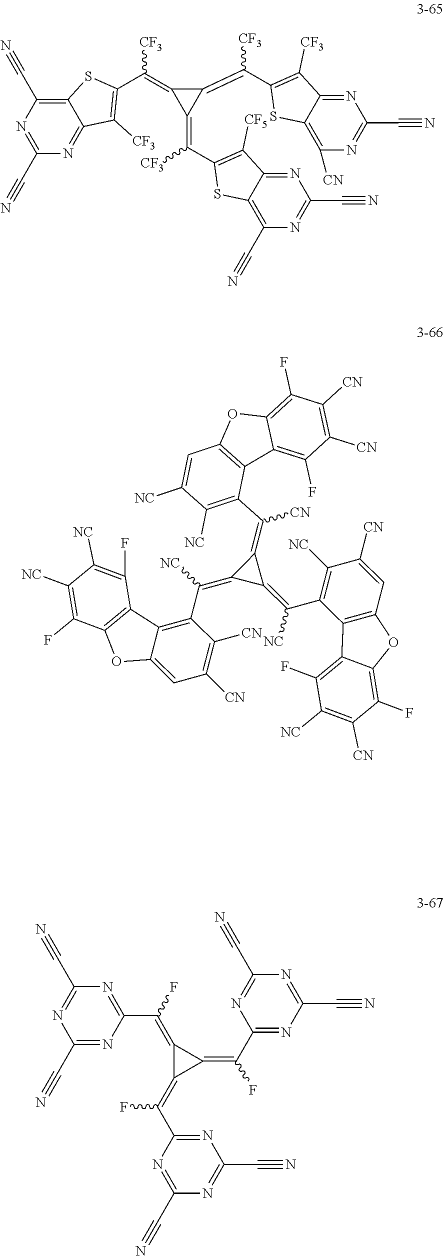

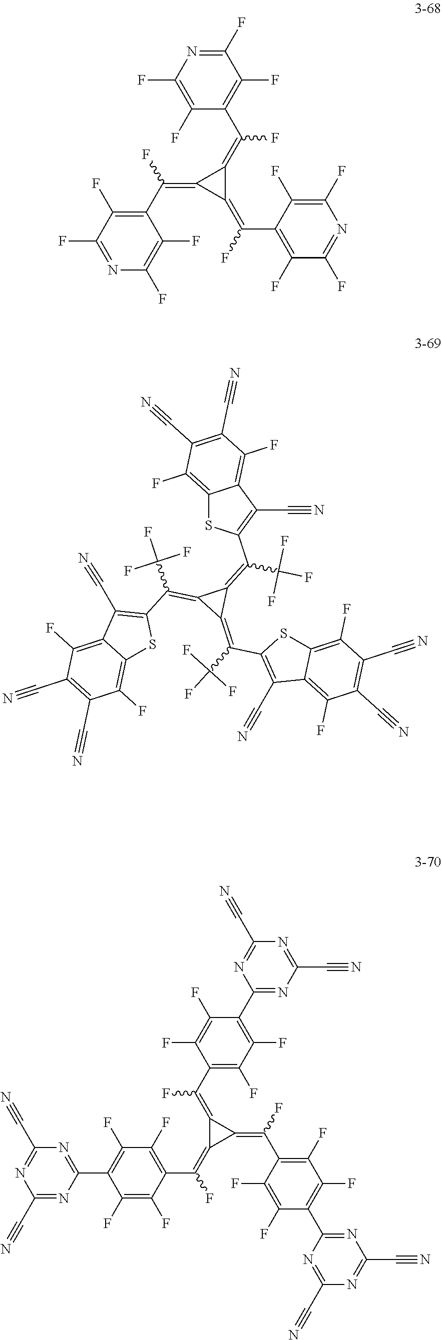 [SMILES: FC(=C1C(=C(F)c2c(F)c(F)nc(F)c2F)C1=C(F)c1c(F)c(F)nc(F)c1F)c1c(F)c(F)nc(F)c1F ; N#Cc1c(C#N)c(F)c2c(C#N)c(C(=C3C(=C(c4sc5c(F)c(C#N)c(C#N)c(F)c5c4C#N)C(F)(F)F)C3=C(c3sc4c(F)c(C#N)c(C#N)c(F)c4c3C#N)C(F)(F)F)C(F)(F)F)sc2c1F ; N#Cc1nc(C#N)nc(-c2c(F)c(F)c(C(F)=C3C(=C(F)c4c(F)c(F)c(-c5nc(C#N)nc(C#N)n5)c(F)c4F)C3=C(F)c3c(F)c(F)c(-c4nc(C#N)nc(C#N)n4)c(F)c3F)c(F)c2F)n1]

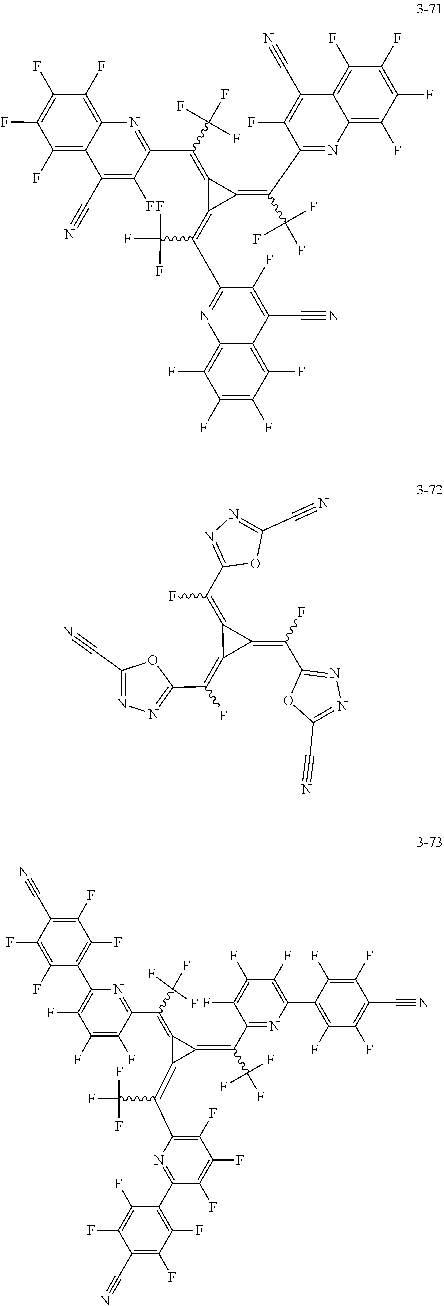 [SMILES: N#Cc1c(F)c(C(=C2C(=C(c3nc4c(F)c(F)c(F)c(F)c4c(C#N)c3F)C(F)(F)F)C2=C(c2nc3c(F)c(F)c(F)c(F)c3c(C#N)c2F)C(F)(F)F)C(F)(F)F)nc2c(F)c(F)c(F)c(F)c12 ; N#Cc1nnc(C(F)=C2C(=C(F)c3nnc(C#N)o3)C2=C(F)c2nnc(C#N)o2)o1 ; N#Cc1c(F)c(F)c(-c2nc(C(=C3C(=C(c4nc(-c5c(F)c(F)c(C#N)c(F)c5F)c(F)c(F)c4F)C(F)(F)F)C3=C(c3nc(-c4c(F)c(F)c(C#N)c(F)c4F)c(F)c(F)c3F)C(F)(F)F)C(F)(F)F)c(F)c(F)c2F)c(F)c1F]

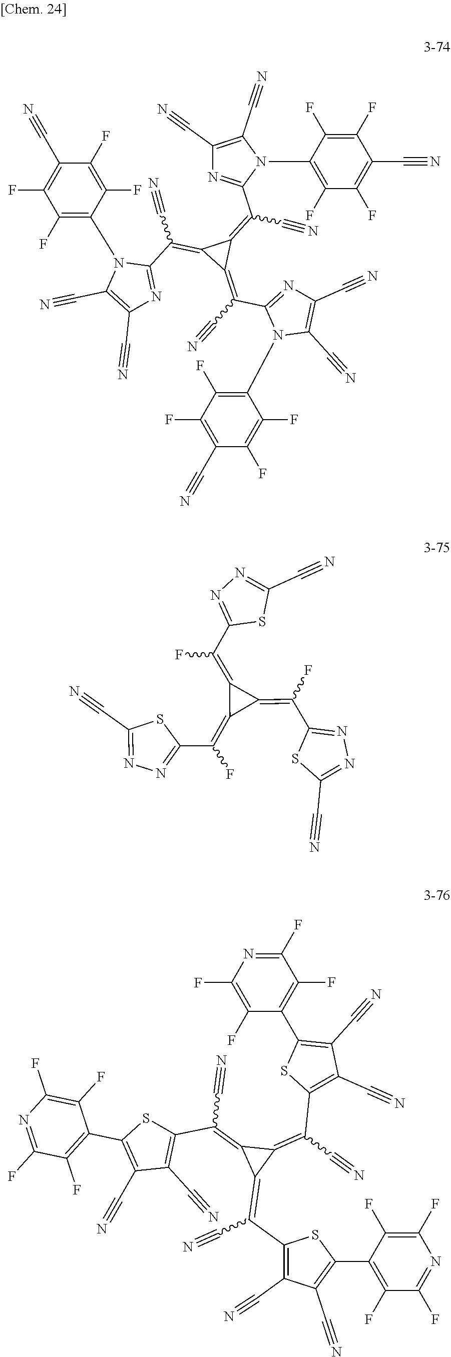 [SMILES: N#CC(=C1C(=C(C#N)c2nc(C#N)c(C#N)n2-c2c(F)c(F)c(C#N)c(F)c2F)C1=C(C#N)c1nc(C#N)c(C#N)n1-c1c(F)c(F)c(C#N)c(F)c1F)c1nc(C#N)c(C#N)n1-c1c(F)c(F)c(C#N)c(F)c1F ; N#Cc1nnc(C(F)=C2C(=C(F)c3nnc(C#N)s3)C2=C(F)c2nnc(C#N)s2)s1 ; N#CC(=C1C(=C(C#N)c2sc(-c3c(F)c(F)nc(F)c3F)c(C#N)c2C#N)C1=C(C#N)c1sc(-c2c(F)c(F)nc(F)c2F)c(C#N)c1C#N)c1sc(-c2c(F)c(F)nc(F)c2F)c(C#N)c1C#N]

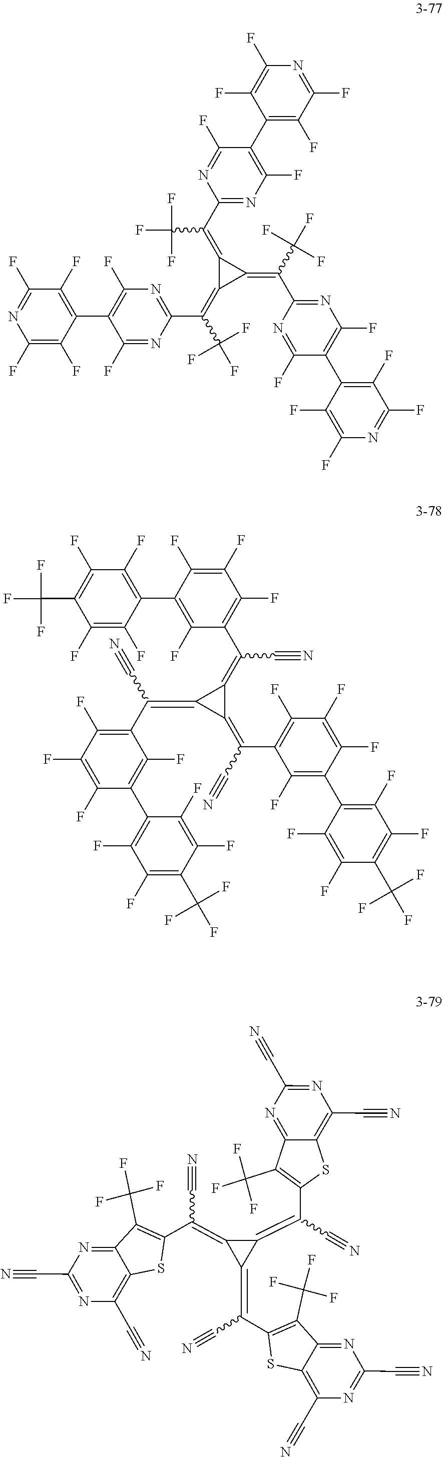 [SMILES: Fc1nc(F)c(F)c(-c2c(F)nc(C(=C3C(=C(c4nc(F)c(-c5c(F)c(F)nc(F)c5F)c(F)n4)C(F)(F)F)C3=C(c3nc(F)c(-c4c(F)c(F)nc(F)c4F)c(F)n3)C(F)(F)F)C(F)(F)F)nc2F)c1F ; N#CC(=C1C(=C(C#N)c2c(F)c(F)c(F)c(-c3c(F)c(F)c(C(F)(F)F)c(F)c3F)c2F)C1=C(C#N)c1c(F)c(F)c(F)c(-c2c(F)c(F)c(C(F)(F)F)c(F)c2F)c1F)c1c(F)c(F)c(F)c(-c2c(F)c(F)c(C(F)(F)F)c(F)c2F)c1F ; N#CC(=C1C(=C(C#N)c2sc3c(C#N)nc(C#N)nc3c2C(F)(F)F)C1=C(C#N)c1sc2c(C#N)nc(C#N)nc2c1C(F)(F)F)c1sc2c(C#N)nc(C#N)nc2c1C(F)(F)F]

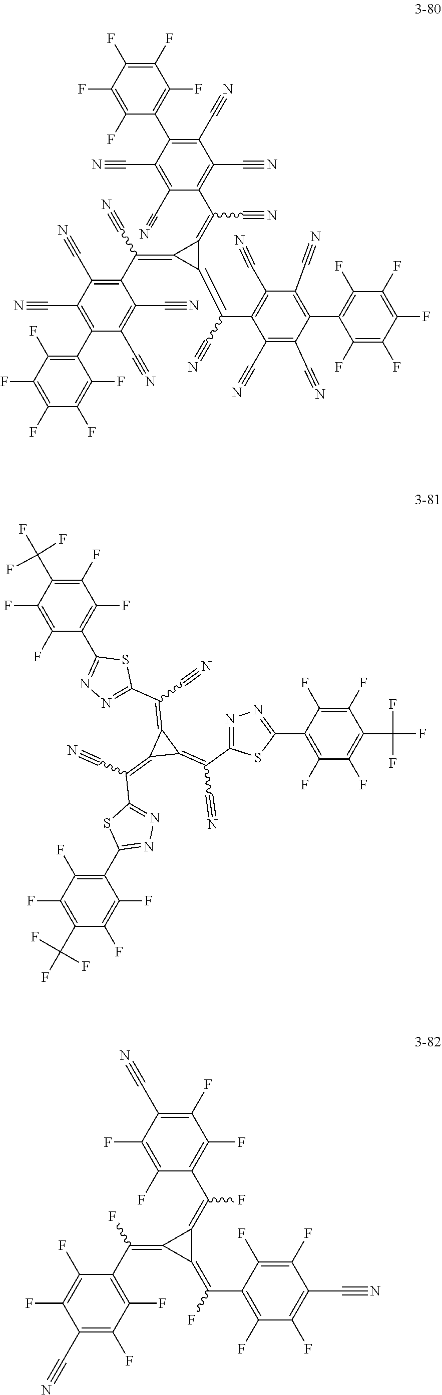 [SMILES: N#CC(=C1C(=C(C#N)c2c(C#N)c(C#N)c(-c3c(F)c(F)c(F)c(F)c3F)c(C#N)c2C#N)C1=C(C#N)c1c(C#N)c(C#N)c(-c2c(F)c(F)c(F)c(F)c2F)c(C#N)c1C#N)c1c(C#N)c(C#N)c(-c2c(F)c(F)c(F)c(F)c2F)c(C#N)c1C#N ; N#CC(=C1C(=C(C#N)c2nnc(-c3c(F)c(F)c(C(F)(F)F)c(F)c3F)s2)C1=C(C#N)c1nnc(-c2c(F)c(F)c(C(F)(F)F)c(F)c2F)s1)c1nnc(-c2c(F)c(F)c(C(F)(F)F)c(F)c2F)s1 ; N#Cc1c(F)c(F)c(C(F)=C2C(=C(F)c3c(F)c(F)c(C#N)c(F)c3F)C2=C(F)c2c(F)c(F)c(C#N)c(F)c2F)c(F)c1F]

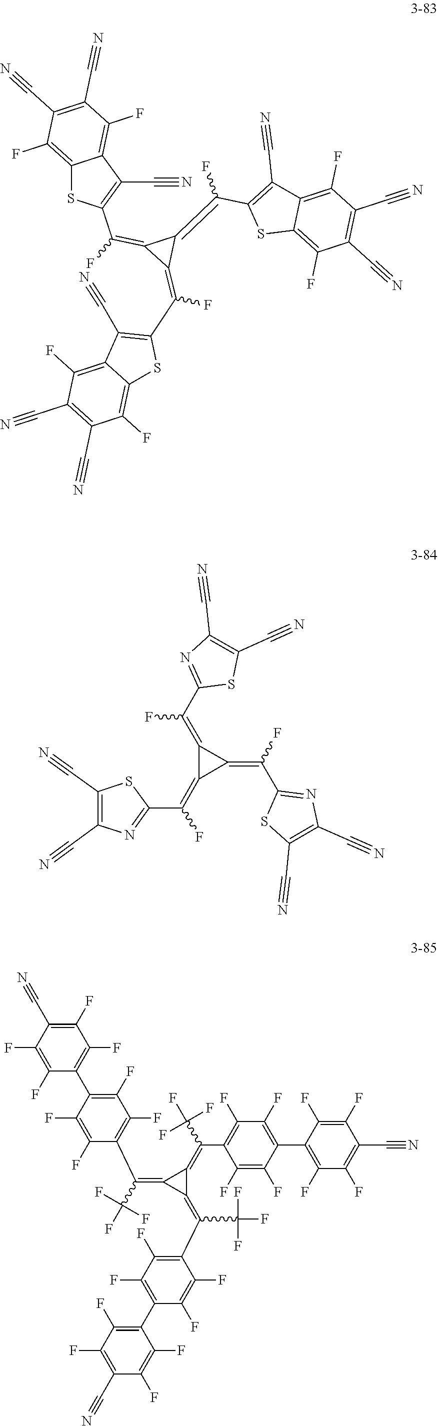 [SMILES: N#Cc1c(C#N)c(F)c2c(C#N)c(C(F)=C3C(=C(F)c4sc5c(F)c(C#N)c(C#N)c(F)c5c4C#N)C3=C(F)c3sc4c(F)c(C#N)c(C#N)c(F)c4c3C#N)sc2c1F ; N#Cc1nc(C(F)=C2C(=C(F)c3nc(C#N)c(C#N)s3)C2=C(F)c2nc(C#N)c(C#N)s2)sc1C#N ; N#Cc1c(F)c(F)c(-c2c(F)c(F)c(C(=C3C(=C(c4c(F)c(F)c(-c5c(F)c(F)c(C#N)c(F)c5F)c(F)c4F)C(F)(F)F)C3=C(c3c(F)c(F)c(-c4c(F)c(F)c(C#N)c(F)c4F)c(F)c3F)C(F)(F)F)C(F)(F)F)c(F)c2F)c(F)c1F]

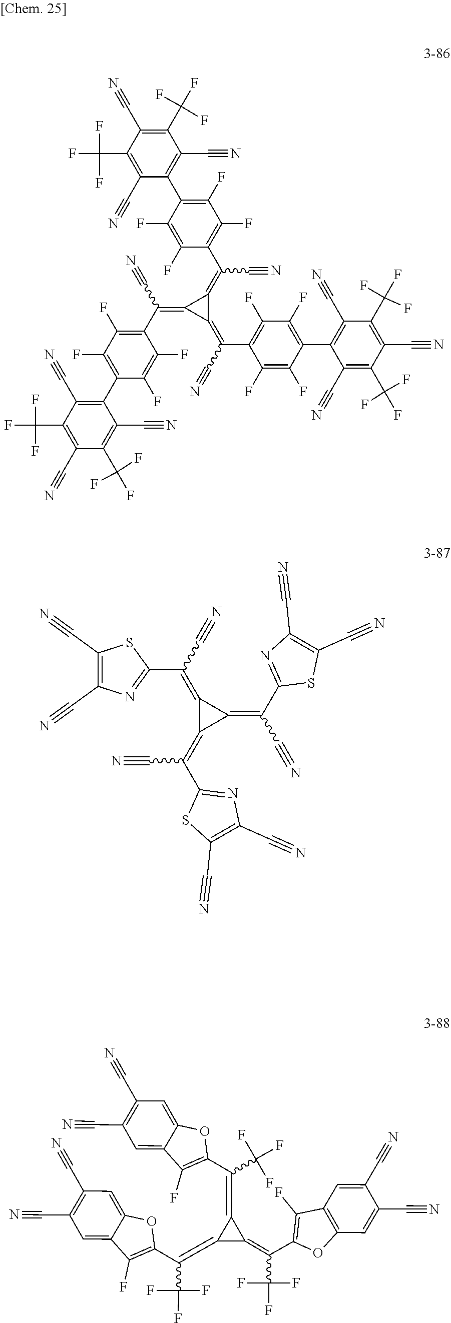

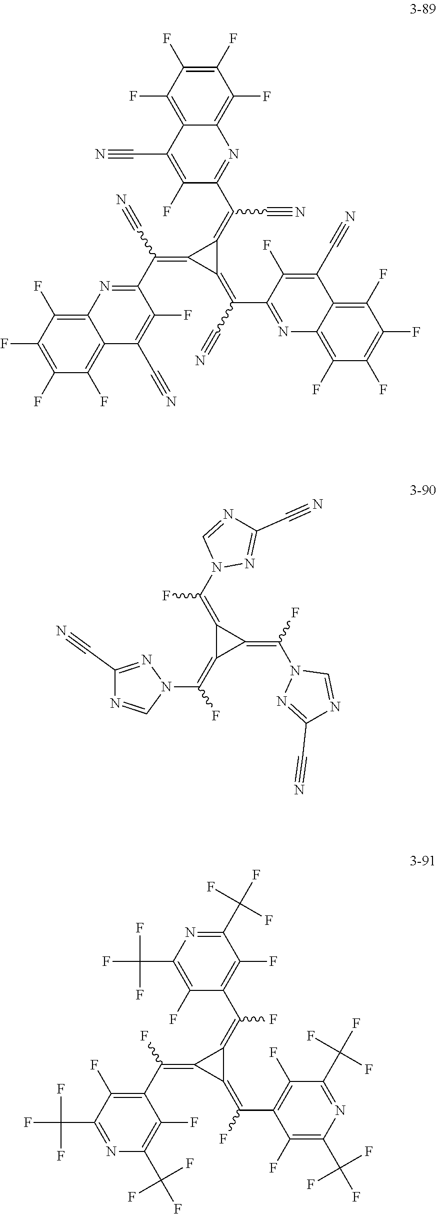 [SMILES: N#CC(=C1C(=C(C#N)c2nc3c(F)c(F)c(F)c(F)c3c(C#N)c2F)C1=C(C#N)c1nc2c(F)c(F)c(F)c(F)c2c(C#N)c1F)c1nc2c(F)c(F)c(F)c(F)c2c(C#N)c1F ; N#Cc1ncn(C(F)=C2C(=C(F)n3cnc(C#N)n3)C2=C(F)n2cnc(C#N)n2)n1 ; FC(=C1C(=C(F)c2c(F)c(C(F)(F)F)nc(C(F)(F)F)c2F)C1=C(F)c1c(F)c(C(F)(F)F)nc(C(F)(F)F)c1F)c1c(F)c(C(F)(F)F)nc(C(F)(F)F)c1F]

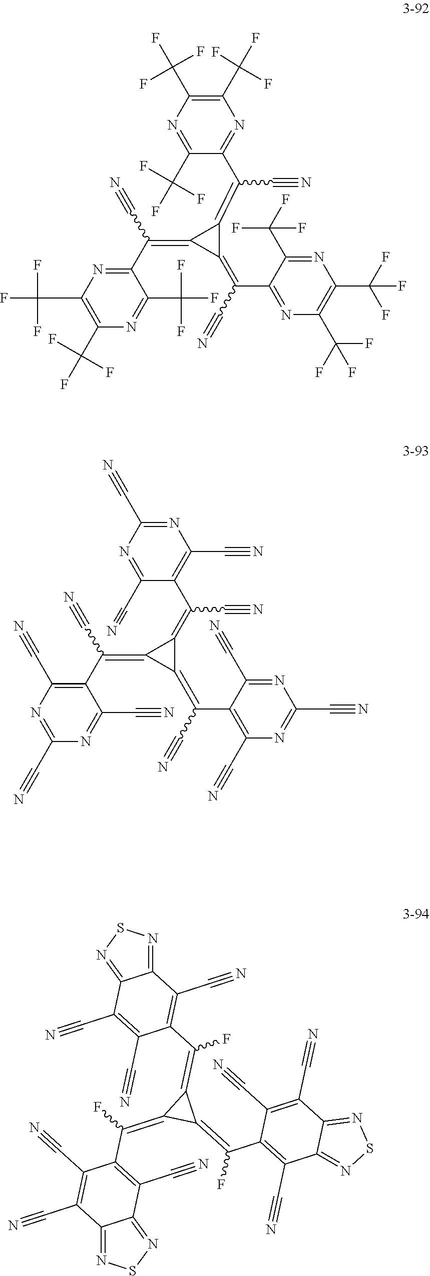 [SMILES: N#CC(=C1C(=C(C#N)c2nc(C(F)(F)F)c(C(F)(F)F)nc2C(F)(F)F)C1=C(C#N)c1nc(C(F)(F)F)c(C(F)(F)F)nc1C(F)(F)F)c1nc(C(F)(F)F)c(C(F)(F)F)nc1C(F)(F)F ; N#CC(=C1C(=C(C#N)c2c(C#N)nc(C#N)nc2C#N)C1=C(C#N)c1c(C#N)nc(C#N)nc1C#N)c1c(C#N)nc(C#N)nc1C#N ; N#Cc1c(C(F)=C2C(=C(F)c3c(C#N)c(C#N)c4nsnc4c3C#N)C2=C(F)c2c(C#N)c(C#N)c3nsnc3c2C#N)c(C#N)c2nsnc2c1C#N]

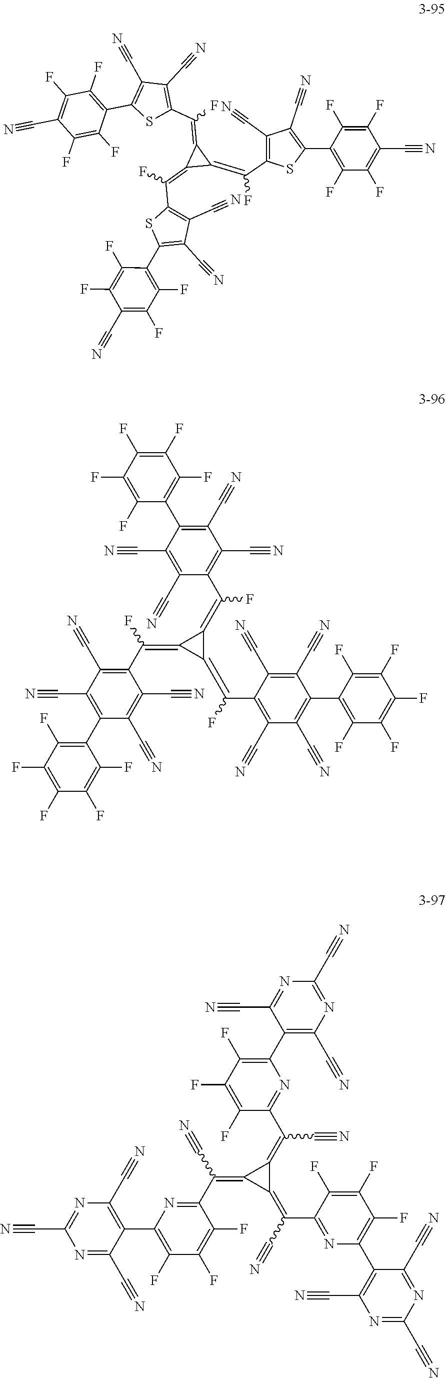

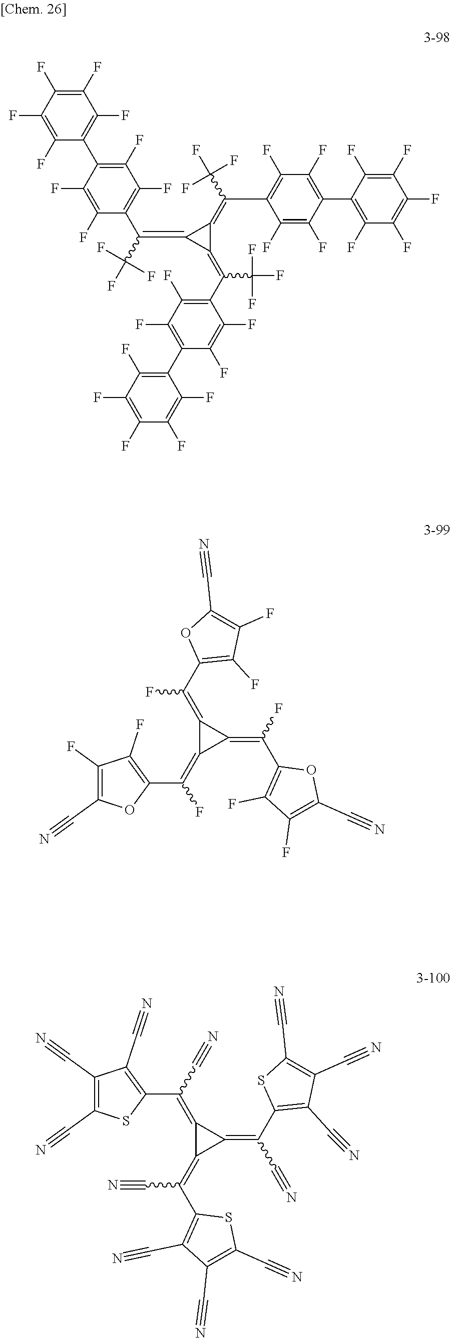 [SMILES: Fc1c(F)c(F)c(-c2c(F)c(F)c(C(=C3C(=C(c4c(F)c(F)c(-c5c(F)c(F)c(F)c(F)c5F)c(F)c4F)C(F)(F)F)C3=C(c3c(F)c(F)c(-c4c(F)c(F)c(F)c(F)c4F)c(F)c3F)C(F)(F)F)C(F)(F)F)c(F)c2F)c(F)c1F ; N#Cc1oc(C(F)=C2C(=C(F)c3oc(C#N)c(F)c3F)C2=C(F)c2oc(C#N)c(F)c2F)c(F)c1F ; N#CC(=C1C(=C(C#N)c2sc(C#N)c(C#N)c2C#N)C1=C(C#N)c1sc(C#N)c(C#N)c1C#N)c1sc(C#N)c(C#N)c1C#N]

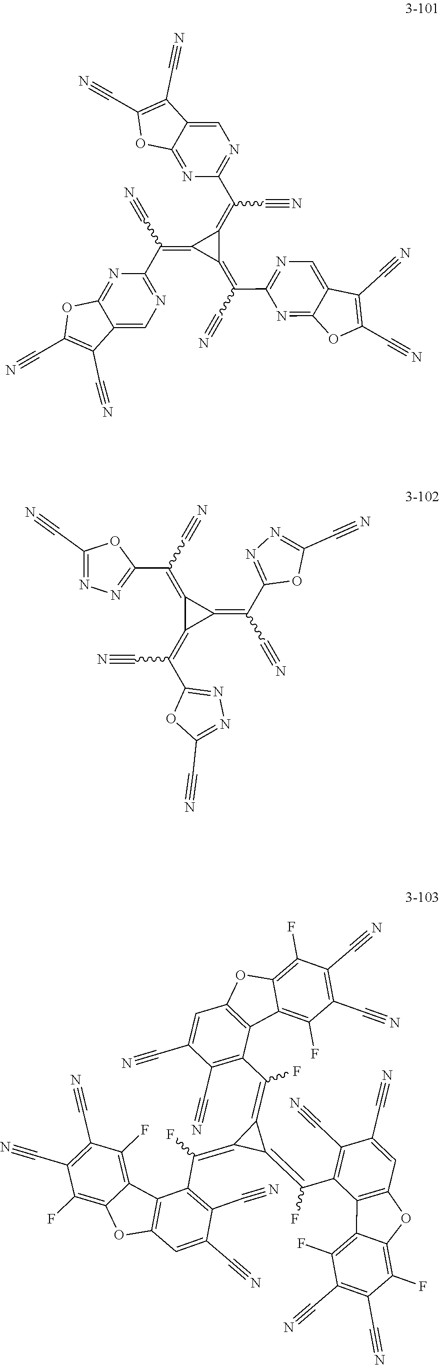 [SMILES: N#CC(=C1C(=C(C#N)c2ncc3c(C#N)c(C#N)oc3n2)C1=C(C#N)c1ncc2c(C#N)c(C#N)oc2n1)c1ncc2c(C#N)c(C#N)oc2n1 ; N#CC(=C1C(=C(C#N)c2nnc(C#N)o2)C1=C(C#N)c1nnc(C#N)o1)c1nnc(C#N)o1 ; N#Cc1cc2oc3c(F)c(C#N)c(C#N)c(F)c3c2c(C(F)=C2C(=C(F)c3c(C#N)c(C#N)cc4oc5c(F)c(C#N)c(C#N)c(F)c5c34)C2=C(F)c2c(C#N)c(C#N)cc3oc4c(F)c(C#N)c(C#N)c(F)c4c23)c1C#N]

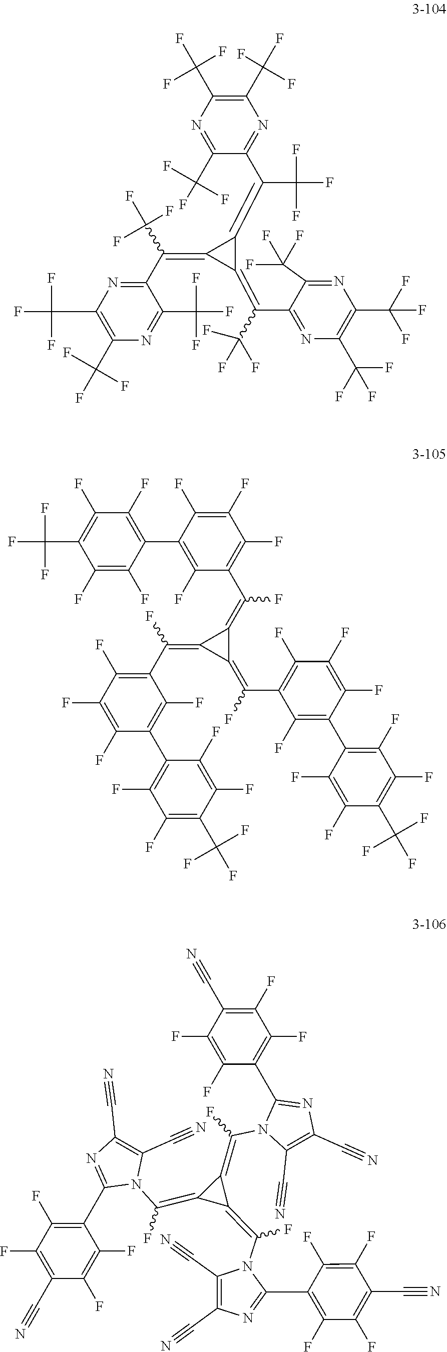 [SMILES: FC(F)(F)C(=C1C(=C(c2nc(C(F)(F)F)c(C(F)(F)F)nc2C(F)(F)F)C(F)(F)F)C1=C(c1nc(C(F)(F)F)c(C(F)(F)F)nc1C(F)(F)F)C(F)(F)F)c1nc(C(F)(F)F)c(C(F)(F)F)nc1C(F)(F)F ; FC(=C1C(=C(F)c2c(F)c(F)c(F)c(-c3c(F)c(F)c(C(F)(F)F)c(F)c3F)c2F)C1=C(F)c1c(F)c(F)c(F)c(-c2c(F)c(F)c(C(F)(F)F)c(F)c2F)c1F)c1c(F)c(F)c(F)c(-c2c(F)c(F)c(C(F)(F)F)c(F)c2F)c1F ; N#Cc1nc(-c2c(F)c(F)c(C#N)c(F)c2F)n(C(F)=C2C(=C(F)n3c(-c4c(F)c(F)c(C#N)c(F)c4F)nc(C#N)c3C#N)C2=C(F)n2c(-c3c(F)c(F)c(C#N)c(F)c3F)nc(C#N)c2C#N)c1C#N]

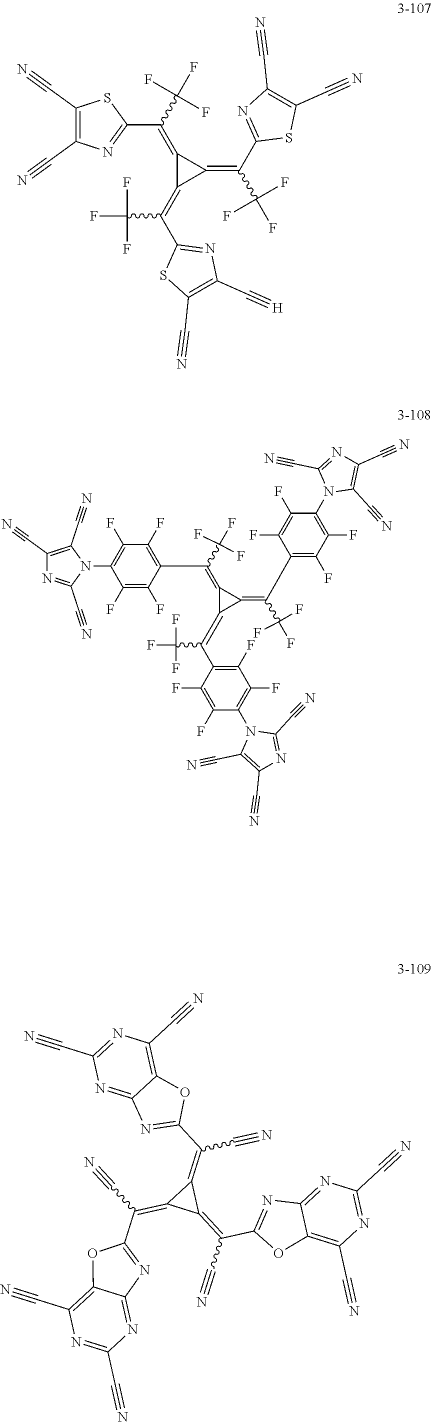

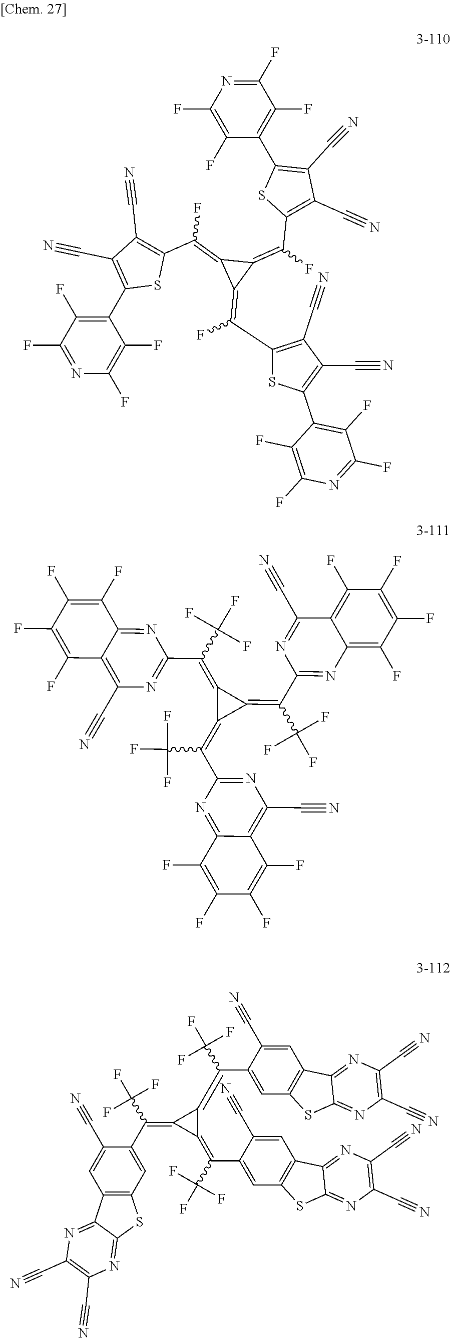 [SMILES: N#Cc1c(C(F)=C2C(=C(F)c3sc(-c4c(F)c(F)nc(F)c4F)c(C#N)c3C#N)C2=C(F)c2sc(-c3c(F)c(F)nc(F)c3F)c(C#N)c2C#N)sc(-c2c(F)c(F)nc(F)c2F)c1C#N ; N#Cc1nc(C(=C2C(=C(c3nc(C#N)c4c(F)c(F)c(F)c(F)c4n3)C(F)(F)F)C2=C(c2nc(C#N)c3c(F)c(F)c(F)c(F)c3n2)C(F)(F)F)C(F)(F)F)nc2c(F)c(F)c(F)c(F)c12 ; N#Cc1cc2c(cc1C(=C1C(=C(c3cc4sc5nc(C#N)c(C#N)nc5c4cc3C#N)C(F)(F)F)C1=C(c1cc3sc4nc(C#N)c(C#N)nc4c3cc1C#N)C(F)(F)F)C(F)(F)F)sc1nc(C#N)c(C#N)nc12]

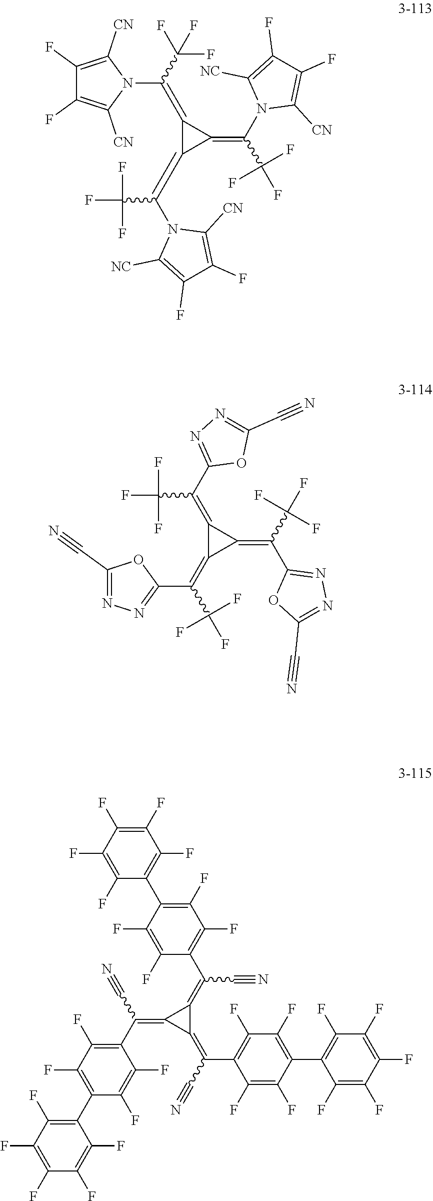 [SMILES: N#Cc1c(F)c(F)c(C#N)n1C(=C1C(=C(n2c(C#N)c(F)c(F)c2C#N)C(F)(F)F)C1=C(n1c(C#N)c(F)c(F)c1C#N)C(F)(F)F)C(F)(F)F ; N#Cc1nnc(C(=C2C(=C(c3nnc(C#N)o3)C(F)(F)F)C2=C(c2nnc(C#N)o2)C(F)(F)F)C(F)(F)F)o1 ; N#CC(=C1C(=C(C#N)c2c(F)c(F)c(-c3c(F)c(F)c(F)c(F)c3F)c(F)c2F)C1=C(C#N)c1c(F)c(F)c(-c2c(F)c(F)c(F)c(F)c2F)c(F)c1F)c1c(F)c(F)c(-c2c(F)c(F)c(F)c(F)c2F)c(F)c1F]

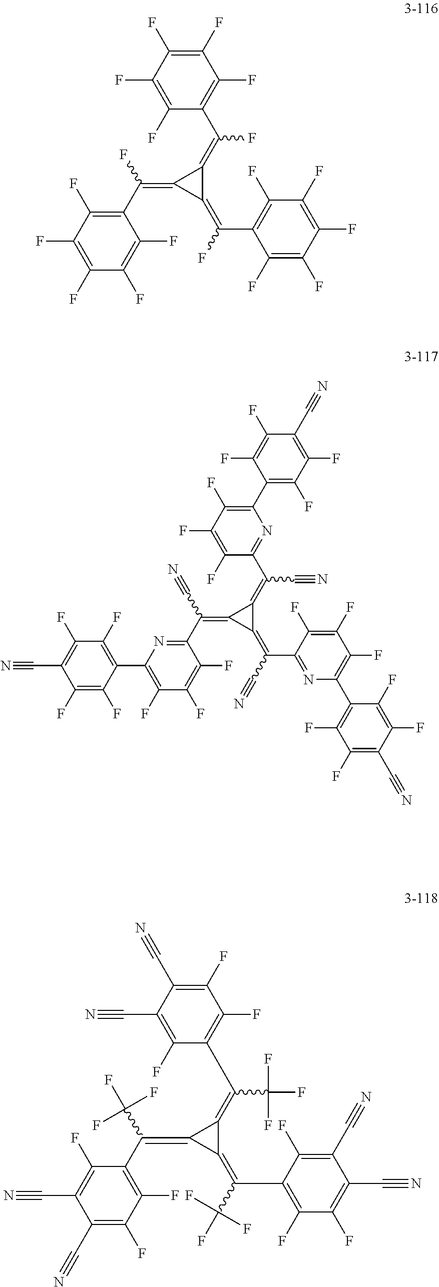 [SMILES: FC(=C1C(=C(F)c2c(F)c(F)c(F)c(F)c2F)C1=C(F)c1c(F)c(F)c(F)c(F)c1F)c1c(F)c(F)c(F)c(F)c1F ; N#CC(=C1C(=C(C#N)c2nc(-c3c(F)c(F)c(C#N)c(F)c3F)c(F)c(F)c2F)C1=C(C#N)c1nc(-c2c(F)c(F)c(C#N)c(F)c2F)c(F)c(F)c1F)c1nc(-c2c(F)c(F)c(C#N)c(F)c2F)c(F)c(F)c1F ; N#Cc1c(F)c(F)c(C(=C2C(=C(c3c(F)c(F)c(C#N)c(C#N)c3F)C(F)(F)F)C2=C(c2c(F)c(F)c(C#N)c(C#N)c2F)C(F)(F)F)C(F)(F)F)c(F)c1C#N]

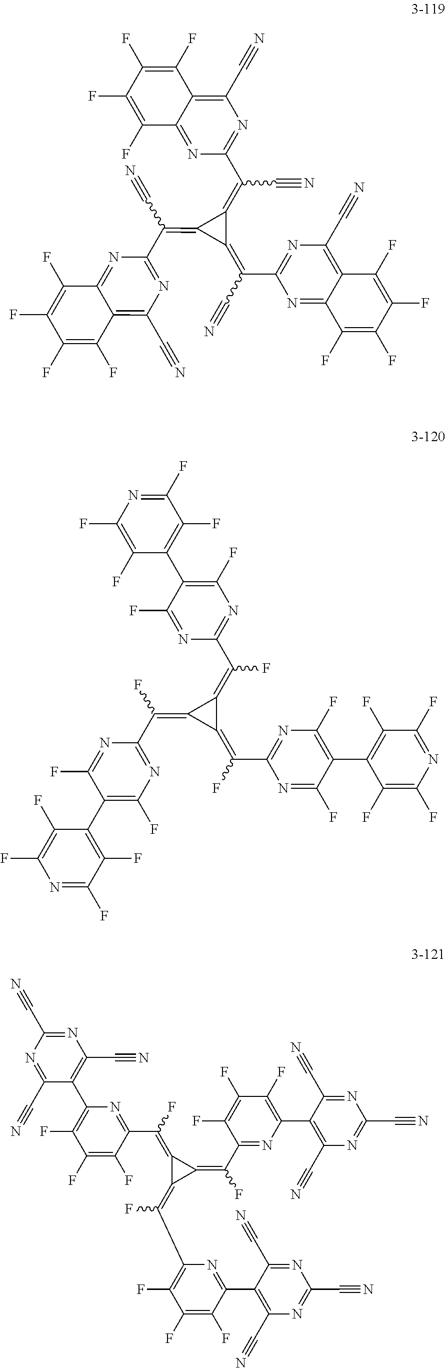

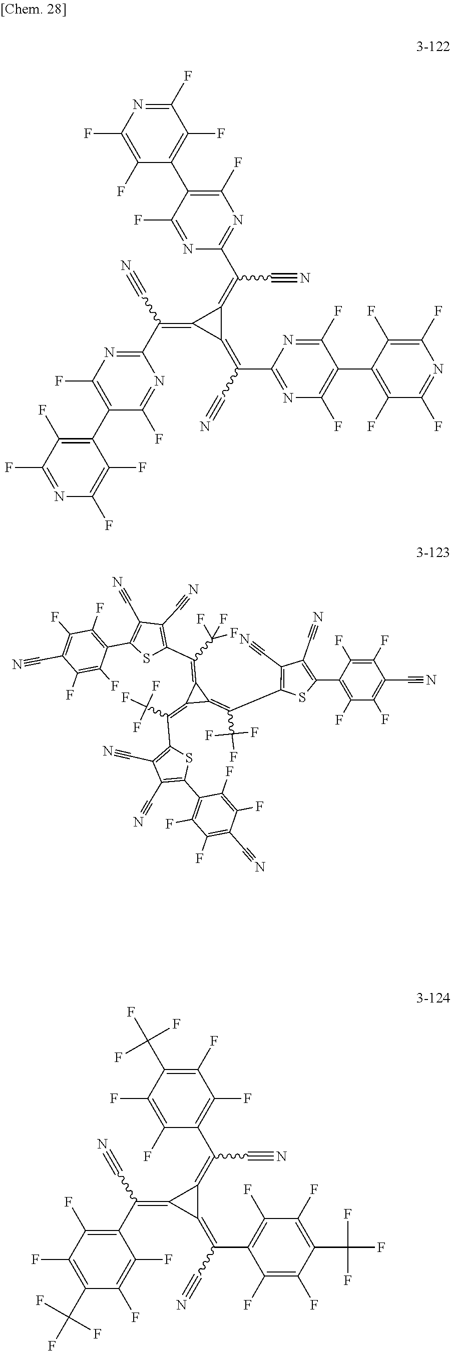 [SMILES: N#CC(=C1C(=C(C#N)c2nc(F)c(-c3c(F)c(F)nc(F)c3F)c(F)n2)C1=C(C#N)c1nc(F)c(-c2c(F)c(F)nc(F)c2F)c(F)n1)c1nc(F)c(-c2c(F)c(F)nc(F)c2F)c(F)n1 ; N#Cc1c(F)c(F)c(-c2sc(C(=C3C(=C(c4sc(-c5c(F)c(F)c(C#N)c(F)c5F)c(C#N)c4C#N)C(F)(F)F)C3=C(c3sc(-c4c(F)c(F)c(C#N)c(F)c4F)c(C#N)c3C#N)C(F)(F)F)C(F)(F)F)c(C#N)c2C#N)c(F)c1F ; N#CC(=C1C(=C(C#N)c2c(F)c(F)c(C(F)(F)F)c(F)c2F)C1=C(C#N)c1c(F)c(F)c(C(F)(F)F)c(F)c1F)c1c(F)c(F)c(C(F)(F)F)c(F)c1F]

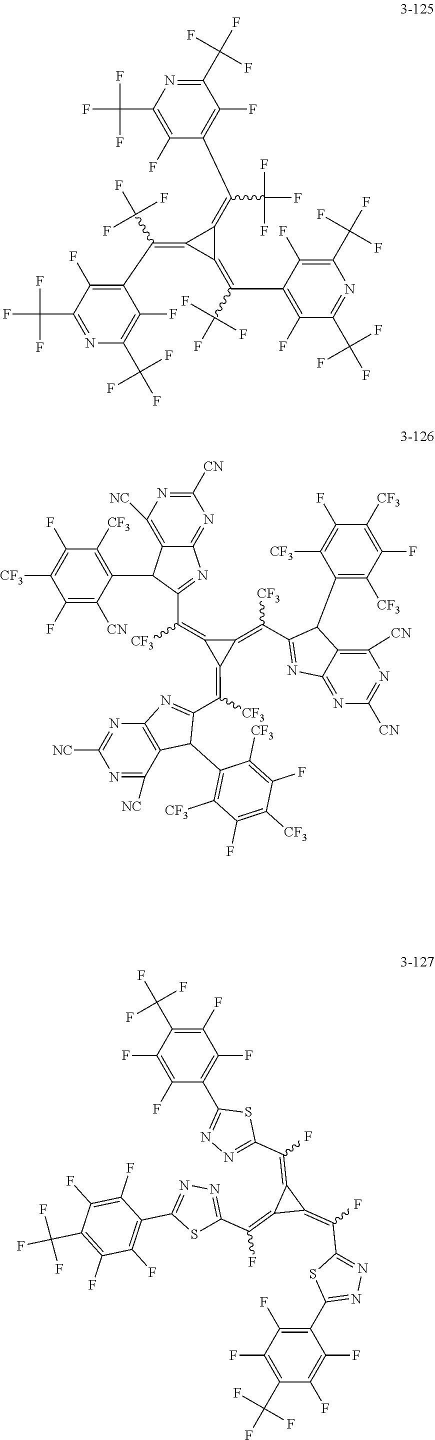

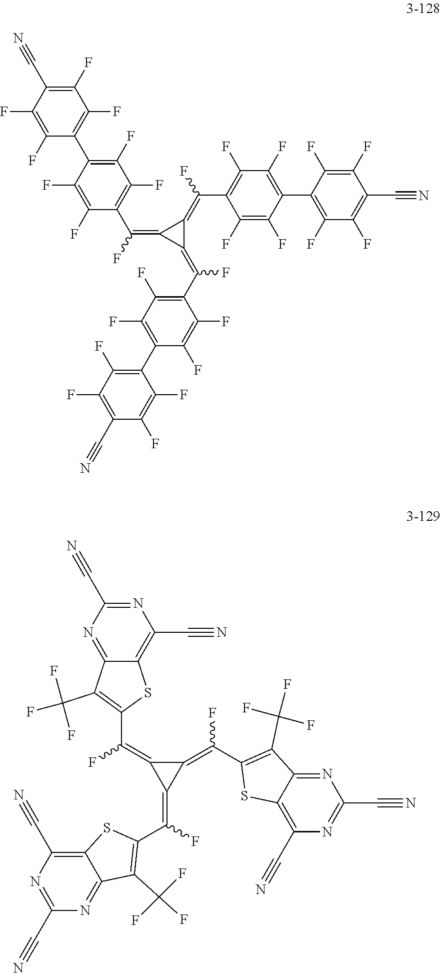 [SMILES: N#Cc1c(F)c(F)c(-c2c(F)c(F)c(C(F)=C3C(=C(F)c4c(F)c(F)c(-c5c(F)c(F)c(C#N)c(F)c5F)c(F)c4F)C3=C(F)c3c(F)c(F)c(-c4c(F)c(F)c(C#N)c(F)c4F)c(F)c3F)c(F)c2F)c(F)c1F ; N#Cc1nc(C#N)c2sc(C(F)=C3C(=C(F)c4sc5c(C#N)nc(C#N)nc5c4C(F)(F)F)C3=C(F)c3sc4c(C#N)nc(C#N)nc4c3C(F)(F)F)c(C(F)(F)F)c2n1]

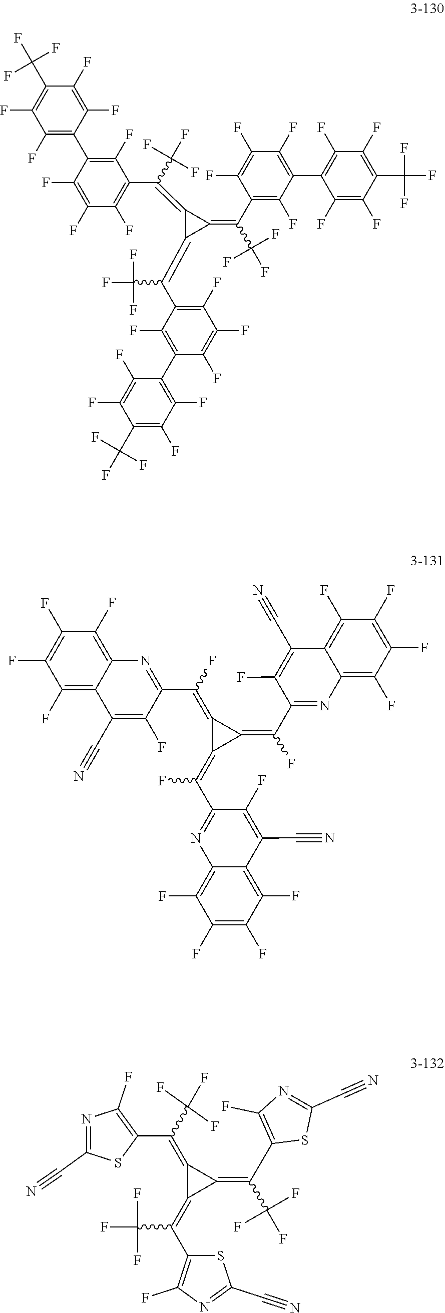 [SMILES: Fc1c(F)c(C(=C2C(=C(c3c(F)c(F)c(F)c(-c4c(F)c(F)c(C(F)(F)F)c(F)c4F)c3F)C(F)(F)F)C2=C(c2c(F)c(F)c(F)c(-c3c(F)c(F)c(C(F)(F)F)c(F)c3F)c2F)C(F)(F)F)C(F)(F)F)c(F)c(-c2c(F)c(F)c(C(F)(F)F)c(F)c2F)c1F ; N#Cc1c(F)c(C(F)=C2C(=C(F)c3nc4c(F)c(F)c(F)c(F)c4c(C#N)c3F)C2=C(F)c2nc3c(F)c(F)c(F)c(F)c3c(C#N)c2F)nc2c(F)c(F)c(F)c(F)c12 ; N#Cc1nc(F)c(C(=C2C(=C(c3sc(C#N)nc3F)C(F)(F)F)C2=C(c2sc(C#N)nc2F)C(F)(F)F)C(F)(F)F)s1]

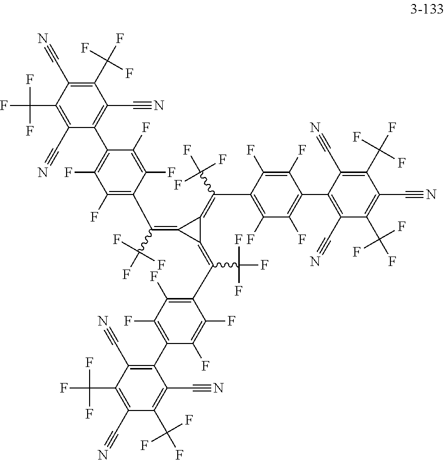 [SMILES: N#Cc1c(-c2c(F)c(F)c(C(=C3C(=C(c4c(F)c(F)c(-c5c(C#N)c(C(F)(F)F)c(C#N)c(C(F)(F)F)c5C#N)c(F)c4F)C(F)(F)F)C3=C(c3c(F)c(F)c(-c4c(C#N)c(C(F)(F)F)c(C#N)c(C(F)(F)F)c4C#N)c(F)c3F)C(F)(F)F)C(F)(F)F)c(F)c2F)c(C#N)c(C(F)(F)F)c(C#N)c1C(F)(F)F]

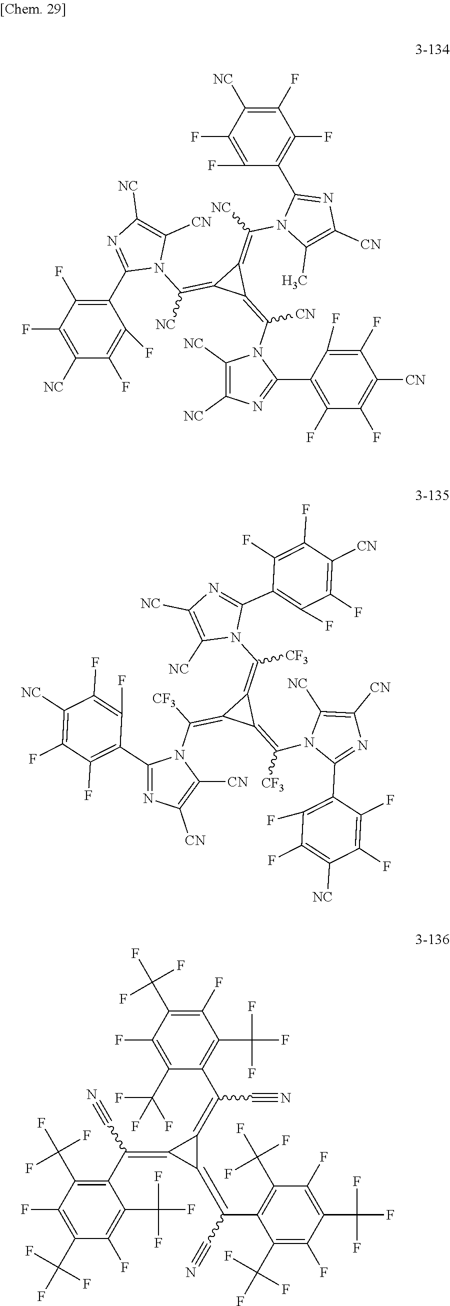 [SMILES: Cc1c(C#N)nc(-c2c(F)c(F)c(C#N)c(F)c2F)n1C(C#N)=C1C(=C(C#N)n2c(-c3c(F)c(F)c(C#N)c(F)c3F)nc(C#N)c2C#N)C1=C(C#N)n1c(-c2c(F)c(F)c(C#N)c(F)c2F)nc(C#N)c1C#N ; N#Cc1nc(-c2c(F)c(F)c(C#N)c(F)c2F)n(C(=C2C(=C(n3c(-c4c(F)c(F)c(C#N)c(F)c4F)nc(C#N)c3C#N)C(F)(F)F)C2=C(n2c(-c3c(F)c(F)c(C#N)c(F)c3F)nc(C#N)c2C#N)C(F)(F)F)C(F)(F)F)c1C#N ; N#CC(=C1C(=C(C#N)c2c(C(F)(F)F)c(F)c(C(F)(F)F)c(F)c2C(F)(F)F)C1=C(C#N)c1c(C(F)(F)F)c(F)c(C(F)(F)F)c(F)c1C(F)(F)F)c1c(C(F)(F)F)c(F)c(C(F)(F)F)c(F)c1C(F)(F)F]

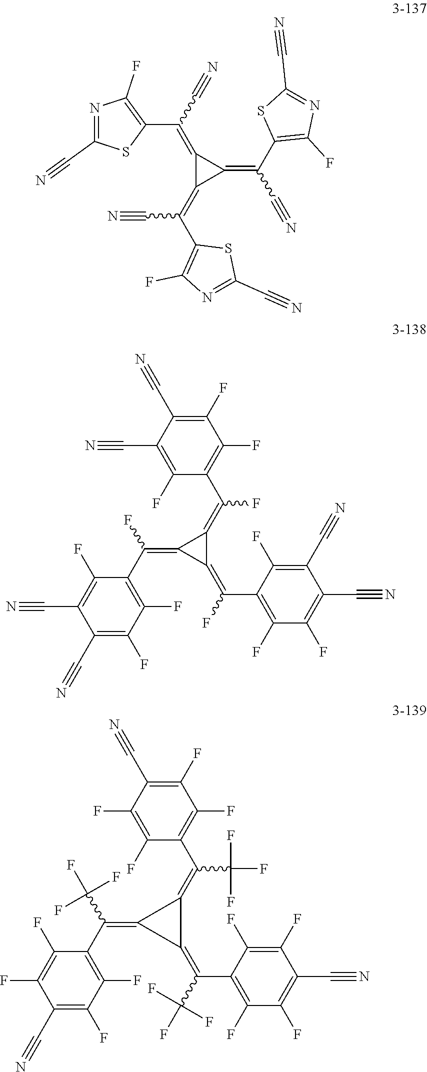 [SMILES: N#CC(=C1C(=C(C#N)c2sc(C#N)nc2F)C1=C(C#N)c1sc(C#N)nc1F)c1sc(C#N)nc1F ; N#Cc1c(F)c(F)c(C(F)=C2C(=C(F)c3c(F)c(F)c(C#N)c(C#N)c3F)C2=C(F)c2c(F)c(F)c(C#N)c(C#N)c2F)c(F)c1C#N ; N#Cc1c(F)c(F)c(C(=C2C(=C(c3c(F)c(F)c(C#N)c(F)c3F)C(F)(F)F)C2=C(c2c(F)c(F)c(C#N)c(F)c2F)C(F)(F)F)C(F)(F)F)c(F)c1F]

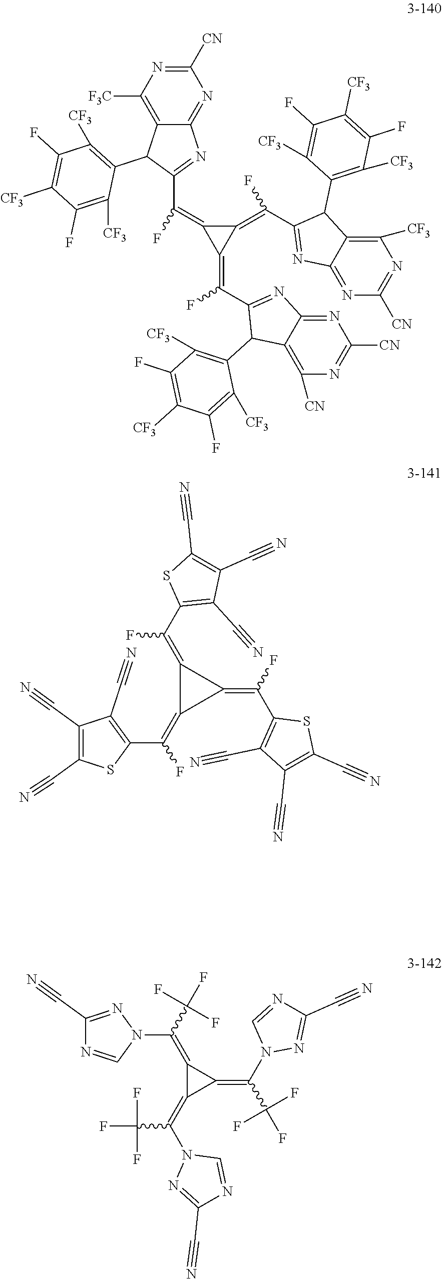 [SMILES: N#Cc1nc(C#N)c2c(n1)N=C(C(F)=C1C(=C(F)C3=Nc4nc(C#N)nc(C(F)(F)F)c4C3c3c(C(F)(F)F)c(F)c(C(F)(F)F)c(F)c3C(F)(F)F)C1=C(F)C1=Nc3nc(C#N)nc(C(F)(F)F)c3C1c1c(C(F)(F)F)c(F)c(C(F)(F)F)c(F)c1C(F)(F)F)C2c1c(C(F)(F)F)c(F)c(C(F)(F)F)c(F)c1C(F)(F)F ; N#Cc1sc(C(F)=C2C(=C(F)c3sc(C#N)c(C#N)c3C#N)C2=C(F)c2sc(C#N)c(C#N)c2C#N)c(C#N)c1C#N ; N#Cc1ncn(C(=C2C(=C(n3cnc(C#N)n3)C(F)(F)F)C2=C(n2cnc(C#N)n2)C(F)(F)F)C(F)(F)F)n1]

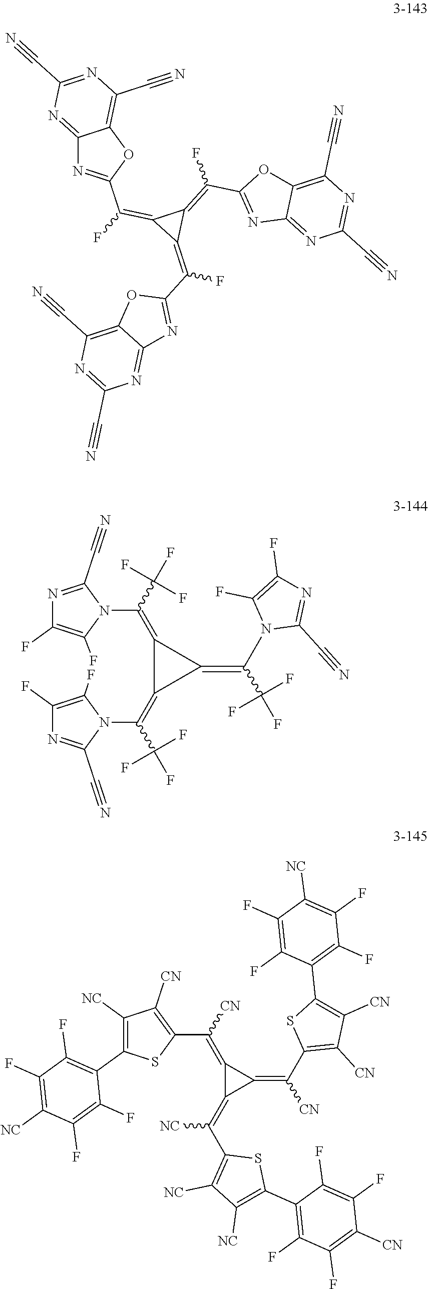 [SMILES: N#Cc1nc(C#N)c2oc(C(F)=C3C(=C(F)c4nc5nc(C#N)nc(C#N)c5o4)C3=C(F)c3nc4nc(C#N)nc(C#N)c4o3)nc2n1 ; N#Cc1nc(F)c(F)n1C(=C1C(=C(n2c(C#N)nc(F)c2F)C(F)(F)F)C1=C(n1c(C#N)nc(F)c1F)C(F)(F)F)C(F)(F)F ; N#CC(=C1C(=C(C#N)c2sc(-c3c(F)c(F)c(C#N)c(F)c3F)c(C#N)c2C#N)C1=C(C#N)c1sc(-c2c(F)c(F)c(C#N)c(F)c2F)c(C#N)c1C#N)c1sc(-c2c(F)c(F)c(C#N)c(F)c2F)c(C#N)c1C#N]

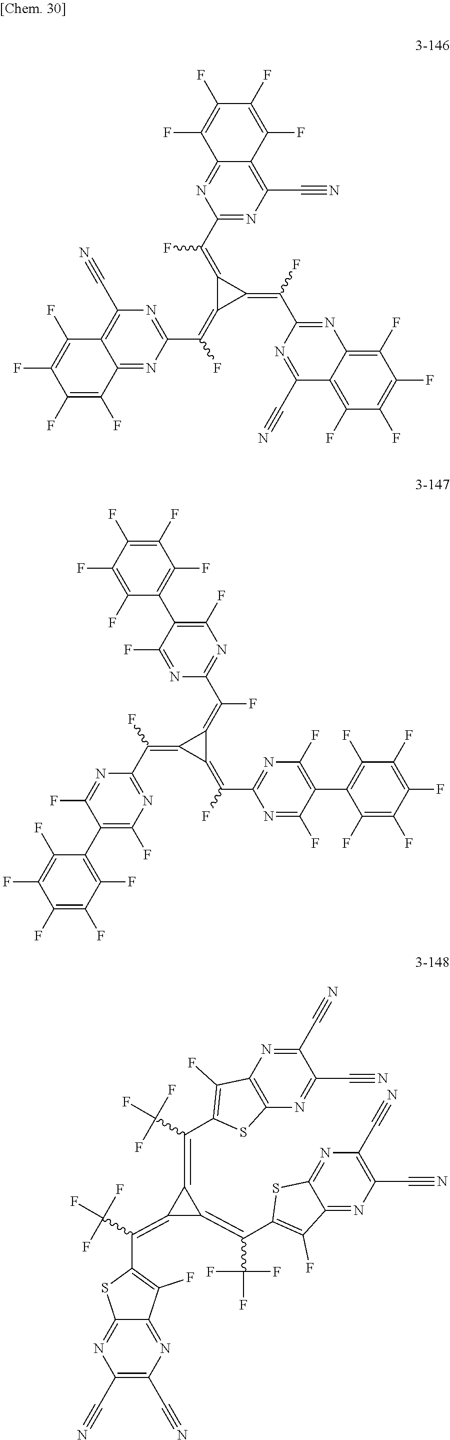 [SMILES: N#Cc1nc(C(F)=C2C(=C(F)c3nc(C#N)c4c(F)c(F)c(F)c(F)c4n3)C2=C(F)c2nc(C#N)c3c(F)c(F)c(F)c(F)c3n2)nc2c(F)c(F)c(F)c(F)c12 ; FC(=C1C(=C(F)c2nc(F)c(-c3c(F)c(F)c(F)c(F)c3F)c(F)n2)C1=C(F)c1nc(F)c(-c2c(F)c(F)c(F)c(F)c2F)c(F)n1)c1nc(F)c(-c2c(F)c(F)c(F)c(F)c2F)c(F)n1 ; N#Cc1nc2sc(C(=C3C(=C(c4sc5nc(C#N)c(C#N)nc5c4F)C(F)(F)F)C3=C(c3sc4nc(C#N)c(C#N)nc4c3F)C(F)(F)F)C(F)(F)F)c(F)c2nc1C#N]

In particular, the radialene compound indicated by formula 3-1 is preferred, especially because it leads to the coexistence of superior drive voltage characteristics, current efficiency characteristics and efficiency change characteristics of the element.

### [Application of the Radialene Compound (3) to the Organic Electroluminescent Element]

The radialene compound (3) according to an aspect of the present disclosure is used in the hole transport region. The hole transport region preferably forms a multilayer structure, which is composed of multiple layers. Preferably, of these layers, the layer containing the radialene compound (3) is used as a layer not contacting the light-emitting layer, more preferably as a layer that functions as a hole injection layer, hole-generating layer or hole transport layer as described above, even more preferably as a hole injection layer or hole-generating layer.

The layer containing the radialene compound (3) may be formed solely of the radialene compound (3) or may be formed of a composition of another compound and the radialene compound (3). It is preferred that the layer be formed of a composition with another compound, particularly because it leads to the demonstration of the superior drive voltage of the element. In that case, it is more preferred that the percentage of the radialene compound be from 0.1% to 20%, even more preferably from 0.5% to 10%, particularly preferably from 0.5% to 5%.

The layer formed by a composition of the radialene compound (3) and another compound may be formed by co-deposition, in which each individual one of the radialene compound (3) and the other compound is heated and simultaneously deposited. Alternatively, a mixture of the radialene compound (3) and the other compound may be heated and deposited. From the viewpoint of controlling the percentage of the radialene compound (3) within the resulting layer, it is preferred to form the layer by co-deposition.

The compound that is deposited together with the radialene compound (3) to form a composition is preferably a compound presented as a specific example for hole injection materials and hole transport materials as mentioned above, more preferably an aromatic tertiary amine compound.

### [Light-Emitting Layer 4]

Between the hole transport region 3 and the later-described electron transport region 5, a light-emitting layer 4 is provided.

Examples of materials for the light-emitting layer include phosphorescent materials, fluorescent materials and thermally activated delayed fluorescent materials. In the light-emitting layer, electron-hole pairs recombine to electrically generate excitons. When these excitons are deactivated, light is produced.

The light-emitting layer 4 may be made of a single small-molecule material or a single polymer material. It is, however, more common for the layer to be made of a host material doped with at least one guest compound. Examples of guest materials include fluorescent compounds, phosphorescent compounds and delayed fluorescent compounds. The emitted light originates primarily from the guest material and can exhibit any color. The light-emitting layer 4, furthermore, can contain a boron compound (1), the details of which will be described later herein.

Examples of host materials include compounds having a biphenyl group, a fluorenyl group, a triphenylsilyl group, a carbazole group, a pyrenyl group, a dibenzofuryl group, a dibenzothienyl group or an anthryl group. More specific examples include DPVBi (4,4′-bis(2,2-diphenylvinyl)-1,1′-biphenyl), BCzVBi (4,4′-bis(9-ethyl-3-carbazovinylene)1,1′-biphenyl), TBADN (2-tert-butyl-9,10-di(2-naphthyl)anthracene), ADN (9,10-di(2-naphthyl)anthracene), CBP (4,4′-bis(carbazol-9-yl)biphenyl), CDBP (4,4′-bis(carbazol-9-yl)-2,2′-dimethylbiphenyl), 2-(9-phenylcarbazole-3-yl)-9-[4-(4-phenylphenylquinazolin-2-yl)carbazole and 9,10-bis(biphenyl)anthracene.

Examples of fluorescent compounds include boron compounds (1), anthracene, pyrene, tetracene, xanthene, perylene, rubrene, coumarin, rhodamine, quinacridone, dicyanomethylenepyran compounds, thiopyran compounds, polymethine compounds, pyrylium, thiapyrylium compounds, fluorene compounds, periflanthene compounds, indenoperylene compounds, bis(azinyl)amine boron compounds, bis(azinyl) methane compounds and carbostyril compounds. The fluorescent dopant may be a combination of two or more selected from such compounds.

Examples of phosphorescent compounds include metal complexes, such as iridium complexes, platinum complexes, palladium complexes and osmium complexes.

Specific examples of fluorescent compounds and phosphorescent compounds include Alq3 (tris(8-hydroxyquinolinolato)aluminum), DPAVBi (4,4′-bis[4-(di-p-tolylamino)styryl]biphenyl), perylene, bis[2-(4-n-hexylphenyl)quinoline](acetylacetonate)iridium (III), Ir(PPy)₃ (tris(2-phenylpyridine)iridium (III)) and FIrPic (bis(3,5-difluoro-2-(2-pyridyl)phenyl-(2-carboxypyridyl)iridium (III))).

Thermally activated delayed fluorescent materials can be used as either host materials or guest materials as described above. Thermally activated delayed fluorescent materials, furthermore, can take on the role of efficiently transferring excitation energy to fluorescent compounds that form the light-emitting layer together with the thermally activated delayed fluorescent materials, without emitting light themselves.

Specific examples of thermally activated delayed fluorescent materials include boron compounds (1), 4Cz-IPN (2,4,5,6-tetra(9-carbazolyl)-isophthalonitrile), 5Cz-BN (2,3,4,5,6-penta(9-carbazolyl)-benzonitrile) and DACTII (2-[3,6-bis(diphenylamino)carbazol-9-ylphenyl]-4,6-diphenyl-1,3,5-triazine).

The light-emitting material, furthermore, is not limited to being contained solely in the light-emitting layer. For example, the light-emitting material may be contained in a layer adjacent to the light-emitting layer (the hole transport layer 5 or the electron transport layer 7). Through this, the current efficiency of the organic electroluminescent element can be further increased.

The light-emitting layer may be in a single-layer structure made of one or two or more materials or may be in a multilayer structure composed of multiple layers with the same composition or different compositions.

### <Boron Compound>

The boron compound according to an aspect of the present disclosure, contained in the light-emitting layer, is indicated by formula (1).

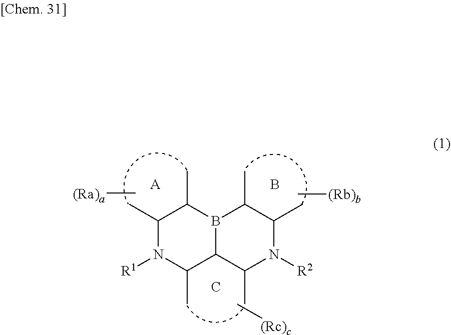

In formula (1),
ring A, ring B and ring C each independently represent
an aromatic hydrocarbon ring in which the number of ring-constituting atoms is from 6 to 20 or
an aromatic heterocycle in which the number of ring-constituting atoms is from 5 to 20,
each optionally having a substituent;
Ra, Rb and Rc each independently represent
   deuterium,
   a cyano group,
   a C1 to C20 alkyl group,
   an aromatic hydrocarbon group in which the number of ring-constituting atoms is from 6 to 20 or
   a heteroaromatic group in which the number of ring-constituting atoms is from 5 to 20;
each of a, b and c is independently an integer of 0 to 4,
ring A and ring B are optionally linked together via an oxygen atom, a sulfur atom, N—R′, Ra or Rb,
R¹, R² and R′ each independently represent
   a C1 to C20 alkyl group,
   an aromatic hydrocarbon group in which the number of ring-constituting atoms is from 6 to 20 or
   a heteroaromatic group in which the number of ring-constituting atoms is from 5 to 20,
   each optionally having a substituent; and

R¹ optionally forms a ring structure by being linked to ring A or ring C via an oxygen atom, a sulfur atom, a substituted or unsubstituted nitrogen atom, Ra or Rc, and
R² optionally forms a ring structure by being linked to ring B or ring C via an oxygen atom, a sulfur atom, a substituted or unsubstituted nitrogen atom, Rb or Rc.

A boron compound indicated by formula (1) may be hereinafter referred to as a compound (1). The ring structures formed in the compound (1), the definitions of substituents and their preferred specific examples are as follows.

### [Rings A to C]

Ring A, ring B and ring C are each independently selected from
an aromatic hydrocarbon ring in which the number of ring-constituting atoms is from 6 to 20 and
an aromatic heterocycle in which the number of ring-constituting atoms is from 5 to 20,
each optionally having a substituent.

Preferably, each of ring A, ring B and ring C is independently an aromatic hydrocarbon ring in which the number of ring-constituting atoms is from 6 to 18 or an aromatic heterocycle in which the number of ring-constituting atoms is from 5 to 18, optionally having a substituent, more preferably an aromatic hydrocarbon ring in which the number of ring-constituting atoms is from 6 to 12 or an aromatic heterocycle in which the number of ring-constituting atoms is from 5 to 12, even more preferably an aromatic hydrocarbon ring in which the number of ring-constituting atoms is from 6 to 9 or an aromatic heterocycle in which the number of ring-constituting atoms is from 5 to 9, more preferably benzene, benzofuran, benzothiophene, indole, benzimidazole, benzothiazole or benzoxazole, particularly preferably benzene.

Ring A and ring B may be linked together via an oxygen atom, a sulfur atom, N—R′ or the later-described Ra or Rb. Ring A and ring B are preferably in a state in which they are linked by N—R′ or not linked. More preferably, ring A and ring B are in a structure in which they are not linked.

### [Ra, Rb and Rc]

Ra, Rb and Rc are each independently selected from
deuterium,
a cyano group,
a C1 to C20 alkyl group,
an aromatic hydrocarbon group in which the number of ring-constituting atoms is from 6 to 20 and
a heteroaromatic group in which the number of ring-constituting atoms is from 5 to 20.

Preferably, each of Ra, Rb and Rc is independently deuterium, a C1 to C20 alkyl group or a heteroaromatic group in which the number of ring-constituting atoms is from 5 to 20, more preferably deuterium or a C1 to C20 alkyl group, even more preferably a C1 to C20 alkyl group.

[a, b and c]

Each of a, b and c is independently an integer of 0 to 4. Preferably, each is independently from 0 to 2. More preferably, each is independently 0 or 1, even more preferably 1.

[R¹, R² and R′]

R¹, R² and R′ each independently represent
a C1 to C20 alkyl group,
an aromatic hydrocarbon group in which the number of ring-constituting atoms is from 6 to 20 or
a heteroaromatic group in which the number of ring-constituting atoms is from 5 to 20,
each optionally having a substituent;

Preferably, each of R¹, R² and R′ is independently an aromatic hydrocarbon group in which the number of ring-constituting atoms is from 6 to 20, optionally having a substituent, or a heteroaromatic group in which the number of ring-constituting atoms is from 5 to 20, optionally having a substituent, more preferably an aromatic hydrocarbon group in which the number of ring-constituting atoms is from 6 to 20, optionally having a substituent, even more preferably an aromatic hydrocarbon group in which the number of ring-constituting atoms is from 6 to 20 substituted with an alkyl group, particularly preferably a phenyl group substituted with an alkyl group.

R¹ may form a ring structure by being linked to ring A or ring C via an oxygen atom, a sulfur atom, a substituted or unsubstituted nitrogen atom, Ra or Rc, and

R² may form a ring structure by being linked to ring B or ring C via an oxygen atom, a sulfur atom, a substituted or unsubstituted nitrogen atom, Rb or Rc.

### [Preferred Examples of Boron Compounds (1)]

A boron compound represented by any one of compounds 1-1 to 1-54 is more preferred because it leads to the coexistence of superior drive voltage characteristics and current efficiency characteristics.

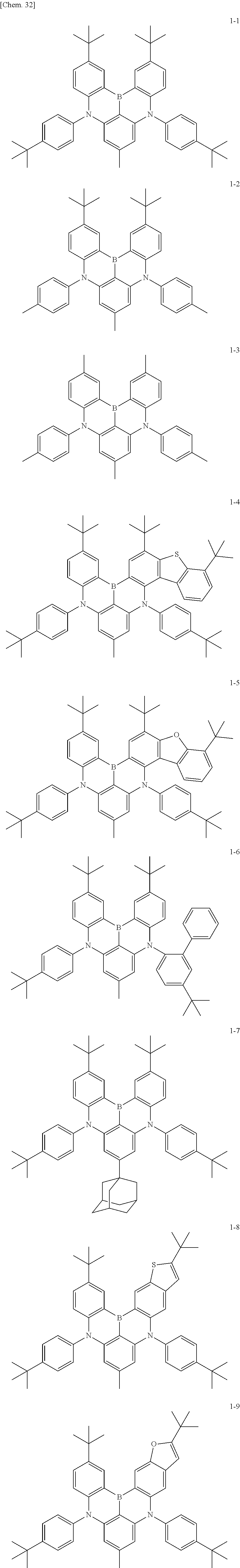

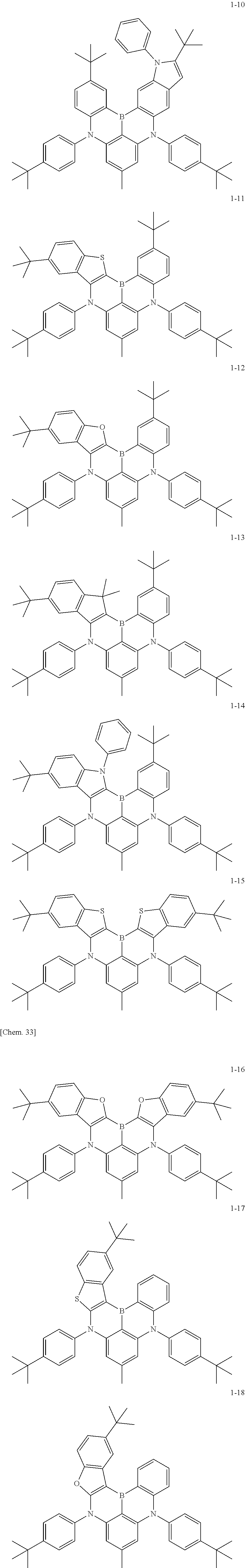

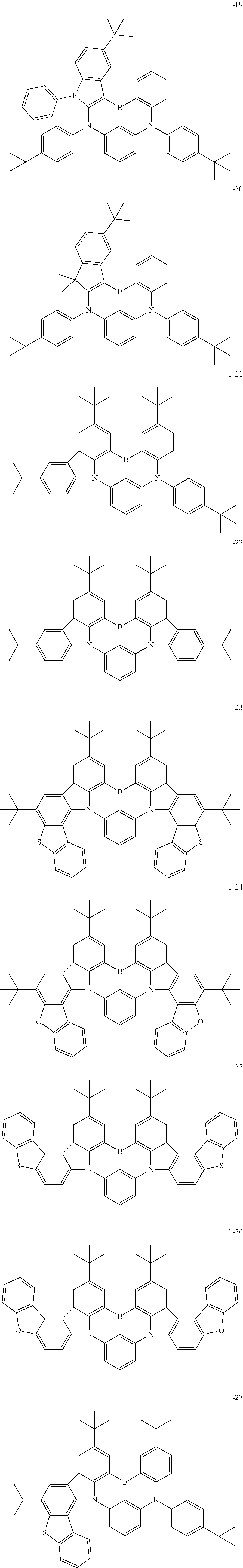

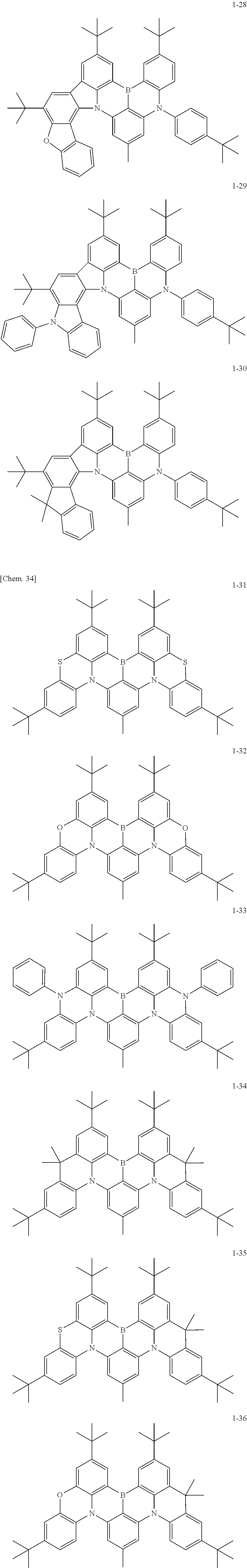

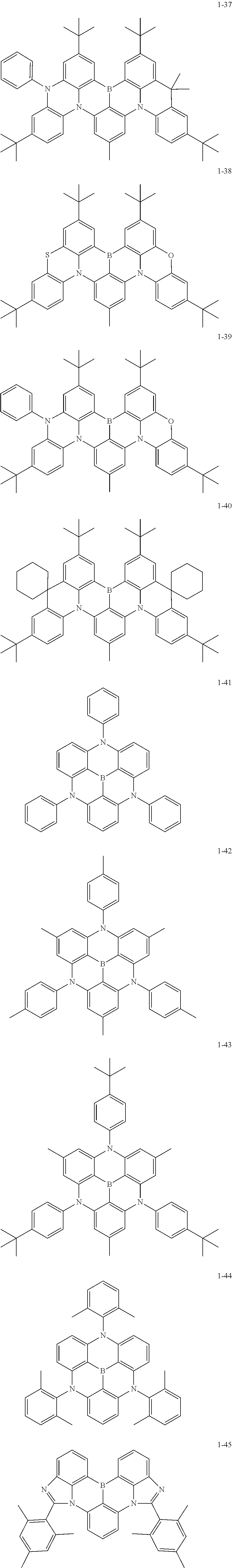

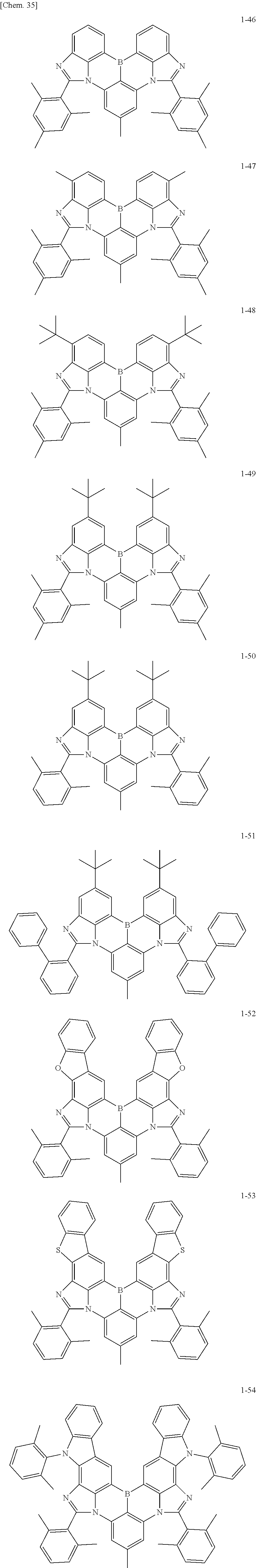

In particular, the boron compounds indicated by 1-1, 1-2, 1-11, 1-12, 1-17, 1-18, 1-21 and 1-22 are preferred, especially because they lead to the coexistence of superior drive voltage characteristics, current efficiency characteristics and efficiency change characteristics of the element. Compounds 1-1, 1-11 and 1-17 are more preferred, and 1-1 is particularly preferred.

### [Electron Transport Region 5]

Between the light-emitting layer 4 and the later-described negative electrode 6, an electron transport layer 5 is provided. It may form a multilayer structure in which a first electron transport layer 51, a second electron transport layer 52 and a third electron transport layer 53 are provided in this order from the positive electrode 2 side.

In the electron transport region 5, a triazine compound represented by formula (2) is contained. The triazine compound may be contained in multiple layers present in the electron transport region.

The first electron transport layer 51, second electron transport layer 52 and third electron transport layer 53 forming the electron transport region 5 each independently have the function(s) of one or more selected from the group consisting of an electron injection layer, an electron-generating layer, an electron transport layer and a hole-blocking layer. For the functions of these layers, the details will be described below.

An electron injection layer has the function of efficiently injecting electrons from the negative electrode 6 or a charge-generating layer and also takes on the function of efficiently injecting electrons into an adjacent layer.

Specific examples of materials for an electron injection layer include metals, such as lithium (Li), potassium (K), cesium (Cs) and ytterbium (Yb), inorganic salts, such as lithium fluoride (LiF), sodium fluoride (NaF), potassium fluoride (KF), lithium carbonate (Li2CO3), sodium carbonate (Na2CO3), potassium carbonate (K2CO3) and cesium carbonate (Cs2CO3), organometallic complexes, such as 8-hydroxyquinolinolatolithium (Liq) and bis(8-hydroxyquinolinolato)zinc, or compositions of such compounds and the triazine compound represented by formula (2).

An electron transport layer takes on the function of transporting electrons injected from a charge-generating layer, the negative electrode or an electron injection layer and injecting electrons into an adjacent layer.

A hole-blocking layer takes on the function of blocking the injection of holes into itself from an adjacent layer. By blocking hole injection, particularly from the light-emitting layer, it allows for an increased total number of electric charges contributing to light emission within the light-emitting layer and, in turn, contributes to enhancing the emission efficiency of the organic electroluminescent element.

An electron transport layer and a hole-blocking layer can contain the triazine compound represented by formula (2). As for specific examples of materials for an electron transport layer and a hole-blocking layer, electron transport materials known in the related art may be contained in addition to the triazine compound represented by formula (2). Examples of electron transport materials known in the related art include 8-hydroxyquinolinolatolithium (Liq), bis(8-hydroxyquinolinolato)zinc, bis(8-hydroxyquinolinolato)copper, bis(8-hydroxyquinolinolato)manganese, tris(8-hydroxyquinolinolato)aluminum, tris(2-methyl-8-hydroxyquinolinolato)aluminum, tris(8-hydroxyquinolinolato)gallium, bis(10-hydroxybenzo[h]quinolinolato)beryllium, bis(10-hydroxybenzo[h]quinolinolato)zinc, bis(2-methyl-8-quinolinolato)chlorogallium, bis(2-methyl-8-quinolinolato)(o-cresolato)gallium, bis(2-methyl-8-quinolinolato)-1-naphtholatoaluminum or bis(2-methyl-8-quinolinolato)-2-naphtholatogallium, 2-[3-(9-phenanthrenyl)-5-(3-pyridinyl)phenyl]-4,6-diphenyl-1,3,5-triazine and 2-(4,″-di-2-pyridinyl[1,1′:3′,1″-terphenyl]-5-yl)-4,6-diphenyl-1,3,5-triazine, BCP (2,9-dimethyl-4,7-diphenyl-1,10-phenanthroline), Bphen (4,7-diphenyl-1,10-phenanthroline), BAlq (bis(2-methyl-8-quinolinolato)-4-(phenylphenolato)aluminum) and bis(10-hydroxybenzo[h]quinolinolato)beryllium) and compositions of such compounds. The material, furthermore, may be a composition resulting from doping such a compound with Liq.

### <Triazine Compound>

The triazine compound according to an aspect of the present disclosure, contained in the electron transport region, is indicated by formula (2):

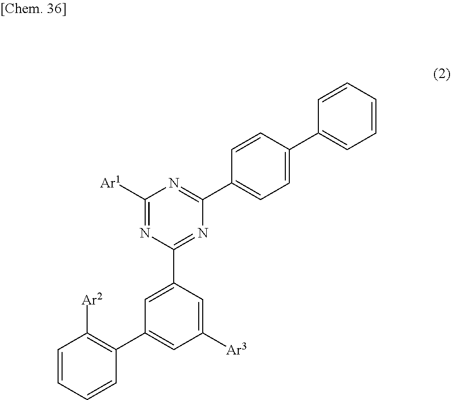

In formula (2),
Ar¹ represents a phenyl group, naphthyl group or biphenylyl group optionally substituted with a C1 to C6 alkyl group, and
Ar² represents a phenyl group, pyridyl group, phenylpyridyl group, pyridylphenyl group or biphenylyl group optionally substituted with a C1 to C4 alkyl group.
Ar³ represents a phenyl group or biphenylyl group optionally substituted with a C1 to C4 alkyl group.

A triazine compound indicated by formula (2) may be hereinafter referred to as a triazine compound (2). The definitions of substituents in the triazine compound (2) and their preferred specific examples are as follows.

### [Ar¹]

Ar¹ is selected from a phenyl group, a naphthyl group and a biphenylyl group.

Ar¹ is preferably a phenyl group or a biphenylyl group because it leads to the coexistence of superior drive voltage characteristics and current efficiency characteristics. A phenyl group or a biphenyl-4-yl group is more preferred.

### [Ar²]

Ar² is selected from a phenyl group, a pyridyl group, a phenylpyridyl group, a pyridylphenyl group and a biphenylyl group, each optionally substituted with a C1 to C4 alkyl group.

Ar² is preferably a phenyl group, a pyridyl group or a biphenylyl group because it leads to the coexistence of superior drive voltage characteristics and current efficiency characteristics. A phenyl group or a biphenylyl group is more preferred, and a phenyl group is particularly preferred.

### [Ar³]

Ar³ is selected from a phenyl group and a biphenylyl group, each optionally substituted with a C1 to C4 alkyl group.

Ar³ is preferably a phenyl group, a biphenyl-2-yl group or a biphenyl-4-yl group because it leads to the coexistence of superior drive voltage characteristics and current efficiency characteristics.

### [Preferred Examples of Triazine Compounds (2)]

The triazine compound (2) that is an aspect of the present disclosure is more preferably a triazine compound represented by any one of compounds 2-1 to 2-80 below.

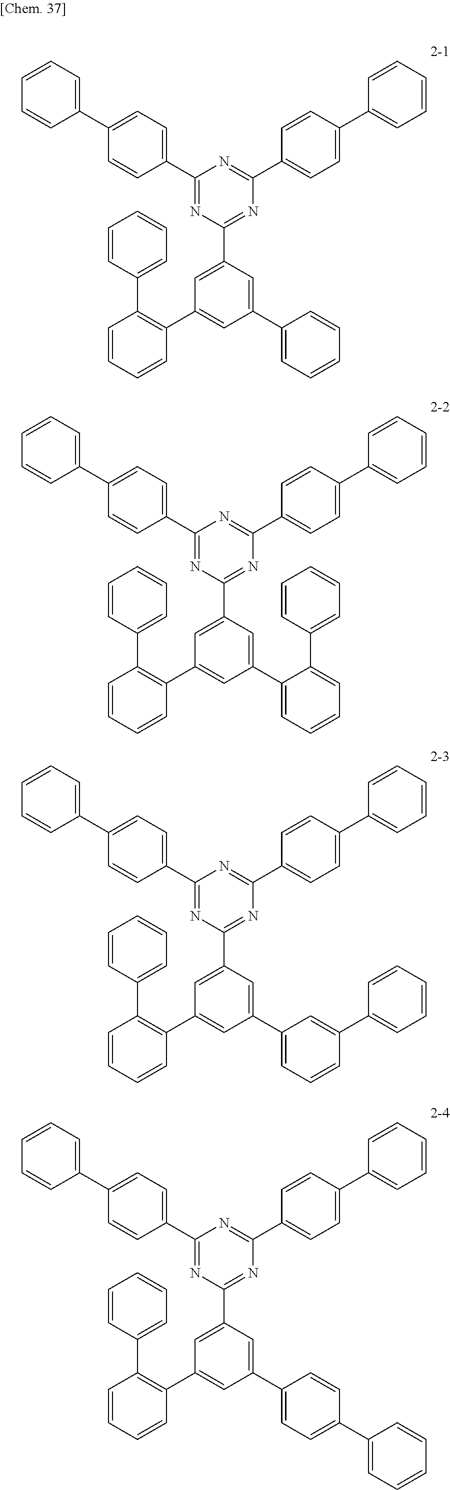 [SMILES: c1ccc(-c2ccc(-c3nc(-c4ccc(-c5ccccc5)cc4)nc(-c4cc(-c5ccccc5)cc(-c5ccccc5-c5ccccc5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5ccccc5)c4)n3)cc2)cc1 ; c1ccc(-c2ccc(-c3nc(-c4ccc(-c5ccccc5)cc4)nc(-c4cc(-c5cccc(-c6ccccc6)c5)cc(-c5ccccc5-c5ccccc5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4ccccc4)c3)cc2)cc1]

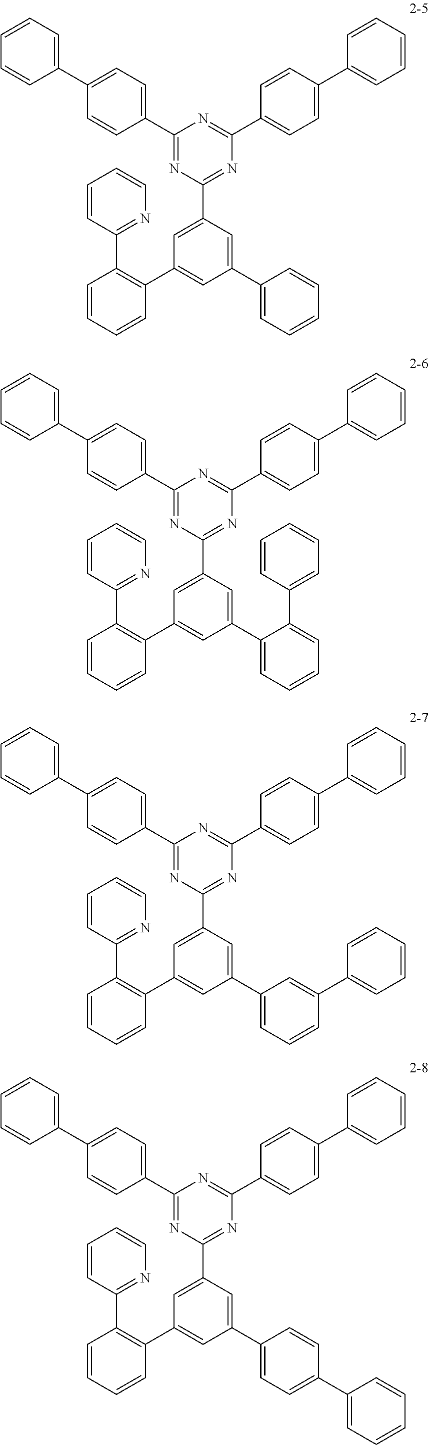 [SMILES: c1ccc(-c2ccc(-c3nc(-c4ccc(-c5ccccc5)cc4)nc(-c4cc(-c5ccccc5)cc(-c5ccccc5-c5ccccn5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5ccccn5)c4)n3)cc2)cc1 ; c1ccc(-c2ccc(-c3nc(-c4ccc(-c5ccccc5)cc4)nc(-c4cc(-c5cccc(-c6ccccc6)c5)cc(-c5ccccc5-c5ccccn5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4ccccn4)c3)cc2)cc1]

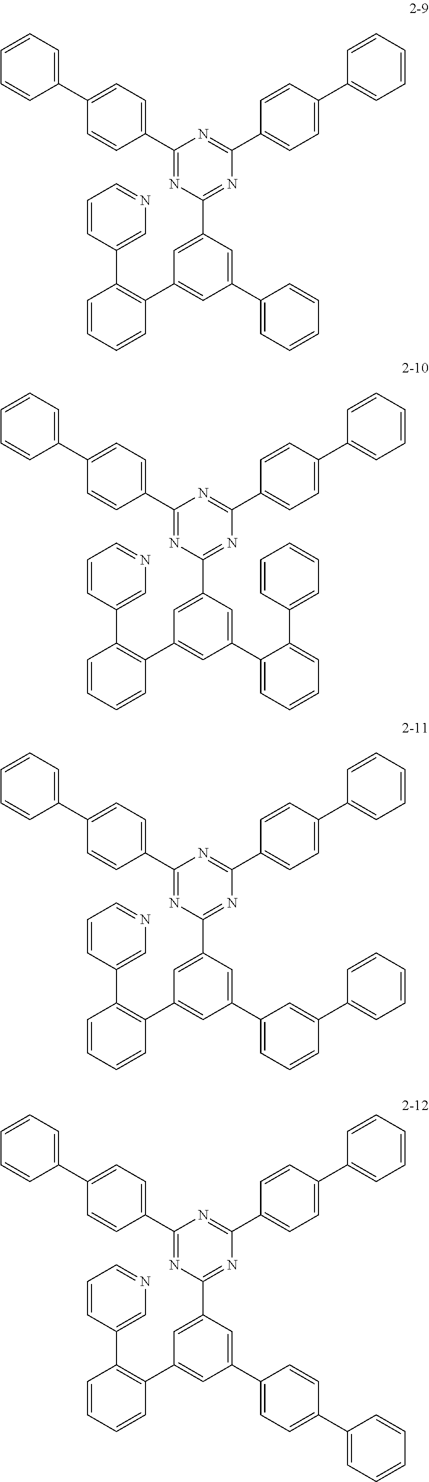 [SMILES: c1ccc(-c2ccc(-c3nc(-c4ccc(-c5ccccc5)cc4)nc(-c4cc(-c5ccccc5)cc(-c5ccccc5-c5cccnc5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5cccnc5)c4)n3)cc2)cc1 ; c1ccc(-c2ccc(-c3nc(-c4ccc(-c5ccccc5)cc4)nc(-c4cc(-c5cccc(-c6ccccc6)c5)cc(-c5ccccc5-c5cccnc5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4cccnc4)c3)cc2)cc1]

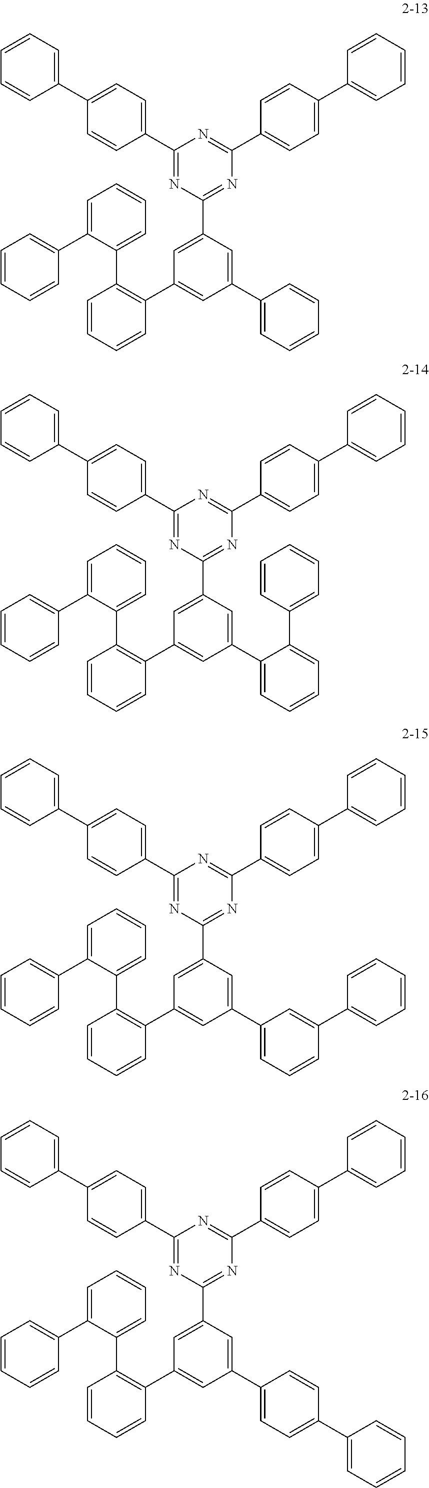 [SMILES: c1ccc(-c2ccc(-c3nc(-c4ccc(-c5ccccc5)cc4)nc(-c4cc(-c5ccccc5)cc(-c5ccccc5-c5ccccc5-c5ccccc5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5ccccc5-c5ccccc5)c4)n3)cc2)cc1 ; c1ccc(-c2ccc(-c3nc(-c4ccc(-c5ccccc5)cc4)nc(-c4cc(-c5cccc(-c6ccccc6)c5)cc(-c5ccccc5-c5ccccc5-c5ccccc5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4ccccc4-c4ccccc4)c3)cc2)cc1]

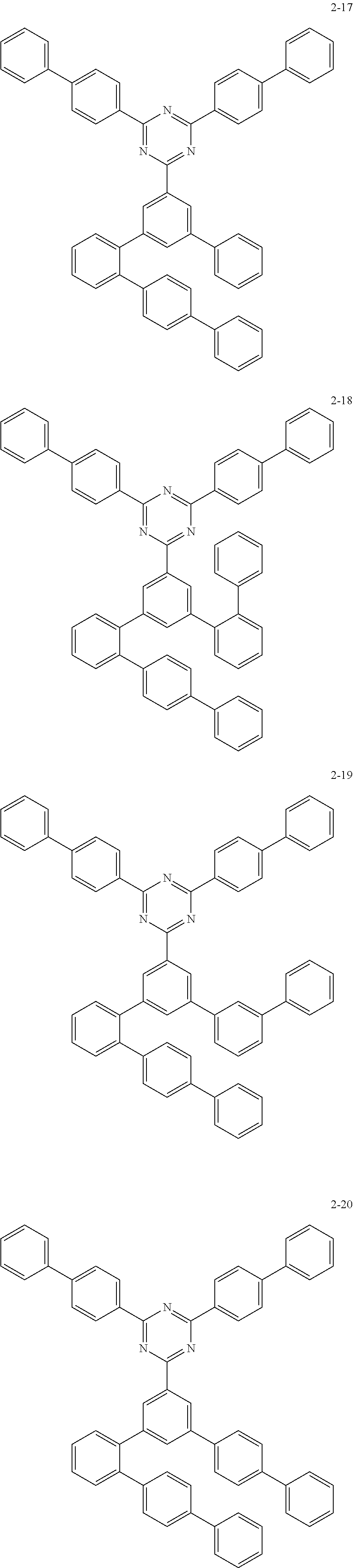 [SMILES: c1ccc(-c2ccc(-c3nc(-c4ccc(-c5ccccc5)cc4)nc(-c4cc(-c5ccccc5)cc(-c5ccccc5-c5ccc(-c6ccccc6)cc5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5ccc(-c6ccccc6)cc5)c4)n3)cc2)cc1 ; c1ccc(-c2ccc(-c3nc(-c4ccc(-c5ccccc5)cc4)nc(-c4cc(-c5cccc(-c6ccccc6)c5)cc(-c5ccccc5-c5ccc(-c6ccccc6)cc5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4ccc(-c5ccccc5)cc4)c3)cc2)cc1]

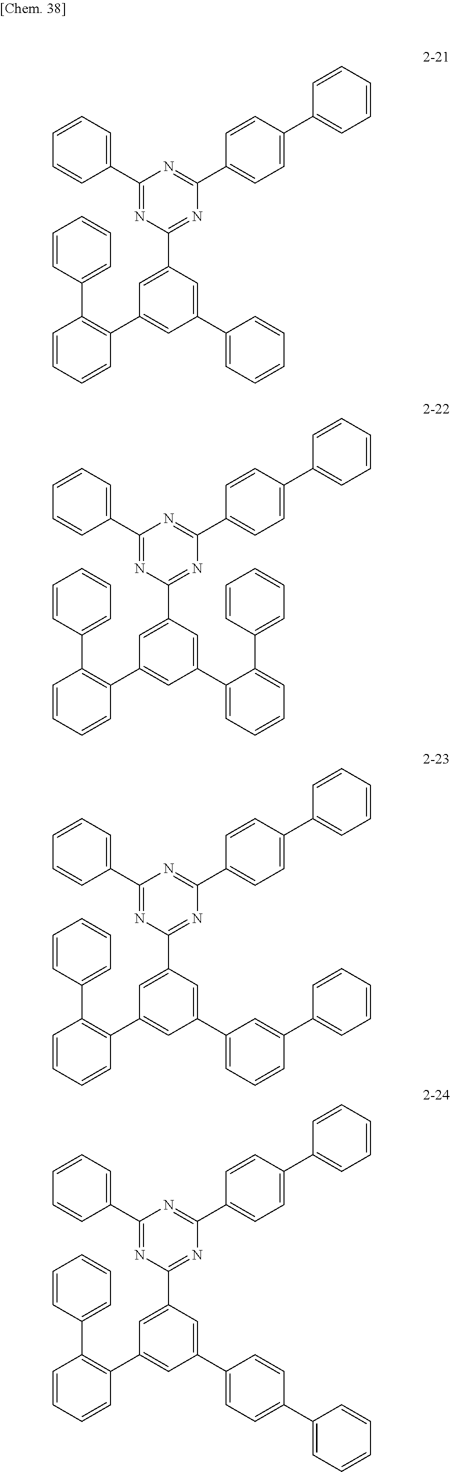 [SMILES: c1ccc(-c2ccc(-c3nc(-c4ccccc4)nc(-c4cc(-c5ccccc5)cc(-c5ccccc5-c5ccccc5)c4)n3)cc2)cc1 ; c1ccc(-c2ccc(-c3nc(-c4ccccc4)nc(-c4cc(-c5ccccc5-c5ccccc5)cc(-c5ccccc5-c5ccccc5)c4)n3)cc2)cc1 ; c1ccc(-c2ccc(-c3nc(-c4ccccc4)nc(-c4cc(-c5cccc(-c6ccccc6)c5)cc(-c5ccccc5-c5ccccc5)c4)n3)cc2)cc1 ; c1ccc(-c2ccc(-c3cc(-c4nc(-c5ccccc5)nc(-c5ccc(-c6ccccc6)cc5)n4)cc(-c4ccccc4-c4ccccc4)c3)cc2)cc1]

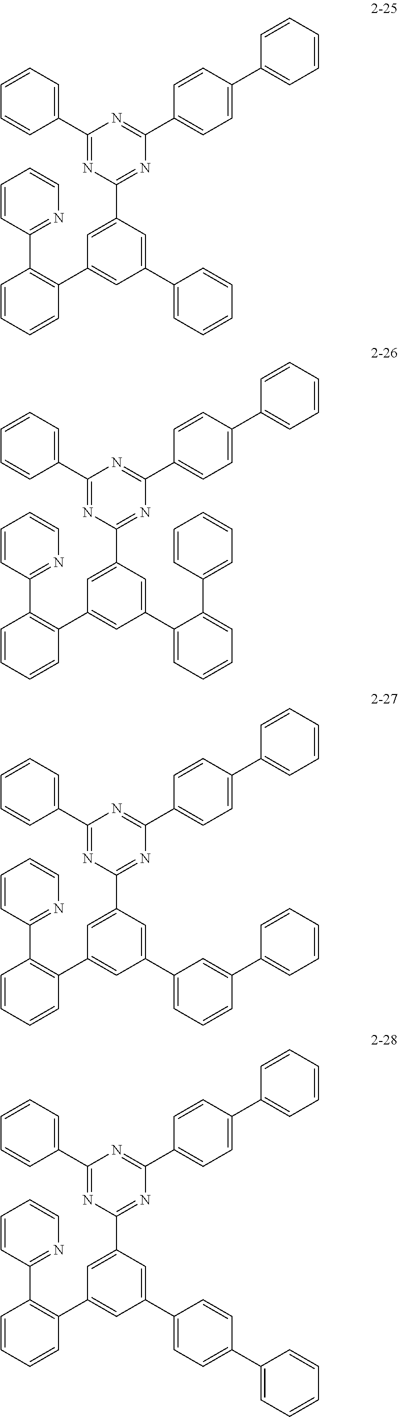 [SMILES: c1ccc(-c2ccc(-c3nc(-c4ccccc4)nc(-c4cc(-c5ccccc5)cc(-c5ccccc5-c5ccccn5)c4)n3)cc2)cc1 ; c1ccc(-c2ccc(-c3nc(-c4ccccc4)nc(-c4cc(-c5ccccc5-c5ccccc5)cc(-c5ccccc5-c5ccccn5)c4)n3)cc2)cc1 ; c1ccc(-c2ccc(-c3nc(-c4ccccc4)nc(-c4cc(-c5cccc(-c6ccccc6)c5)cc(-c5ccccc5-c5ccccn5)c4)n3)cc2)cc1 ; c1ccc(-c2ccc(-c3cc(-c4nc(-c5ccccc5)nc(-c5ccc(-c6ccccc6)cc5)n4)cc(-c4ccccc4-c4ccccn4)c3)cc2)cc1]

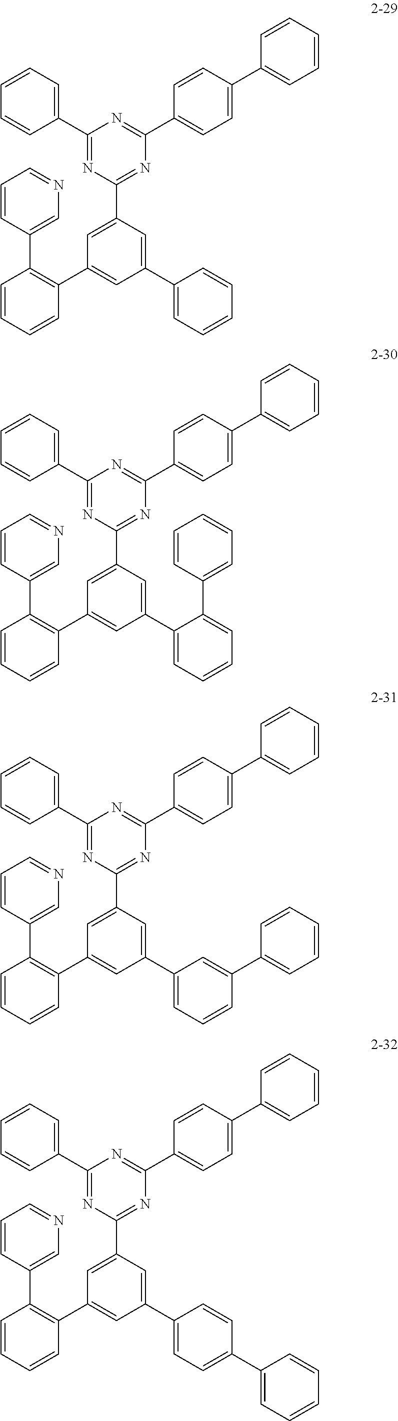 [SMILES: c1ccc(-c2ccc(-c3nc(-c4ccccc4)nc(-c4cc(-c5ccccc5)cc(-c5ccccc5-c5cccnc5)c4)n3)cc2)cc1 ; c1ccc(-c2ccc(-c3nc(-c4ccccc4)nc(-c4cc(-c5ccccc5-c5ccccc5)cc(-c5ccccc5-c5cccnc5)c4)n3)cc2)cc1 ; c1ccc(-c2ccc(-c3nc(-c4ccccc4)nc(-c4cc(-c5cccc(-c6ccccc6)c5)cc(-c5ccccc5-c5cccnc5)c4)n3)cc2)cc1 ; c1ccc(-c2ccc(-c3cc(-c4nc(-c5ccccc5)nc(-c5ccc(-c6ccccc6)cc5)n4)cc(-c4ccccc4-c4cccnc4)c3)cc2)cc1]

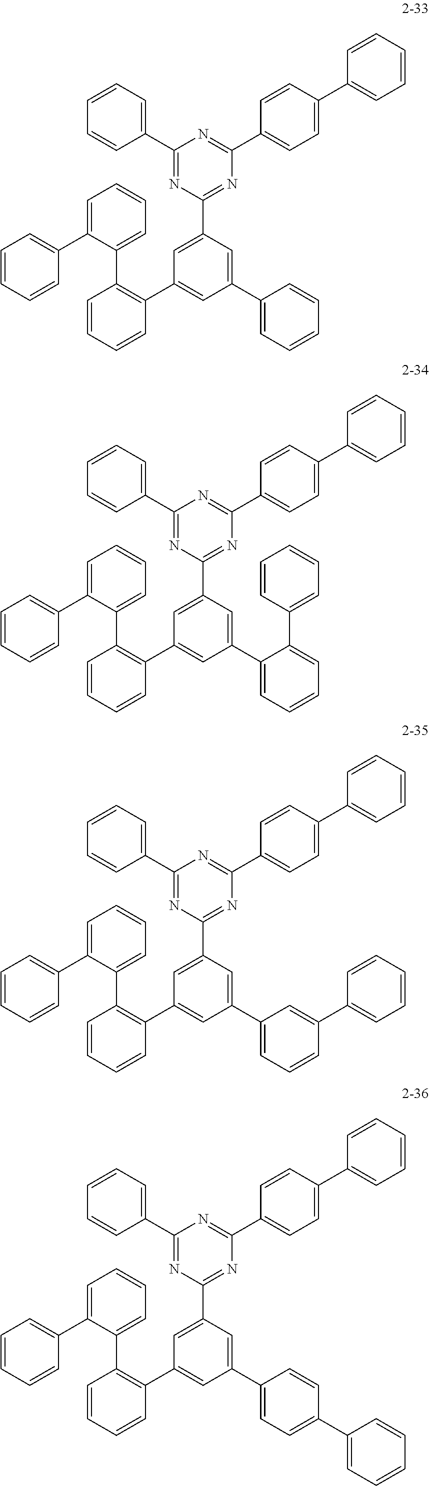 [SMILES: c1ccc(-c2ccc(-c3nc(-c4ccccc4)nc(-c4cc(-c5ccccc5)cc(-c5ccccc5-c5ccccc5-c5ccccc5)c4)n3)cc2)cc1 ; c1ccc(-c2ccc(-c3nc(-c4ccccc4)nc(-c4cc(-c5ccccc5-c5ccccc5)cc(-c5ccccc5-c5ccccc5-c5ccccc5)c4)n3)cc2)cc1 ; c1ccc(-c2ccc(-c3nc(-c4ccccc4)nc(-c4cc(-c5cccc(-c6ccccc6)c5)cc(-c5ccccc5-c5ccccc5-c5ccccc5)c4)n3)cc2)cc1 ; c1ccc(-c2ccc(-c3cc(-c4nc(-c5ccccc5)nc(-c5ccc(-c6ccccc6)cc5)n4)cc(-c4ccccc4-c4ccccc4-c4ccccc4)c3)cc2)cc1]

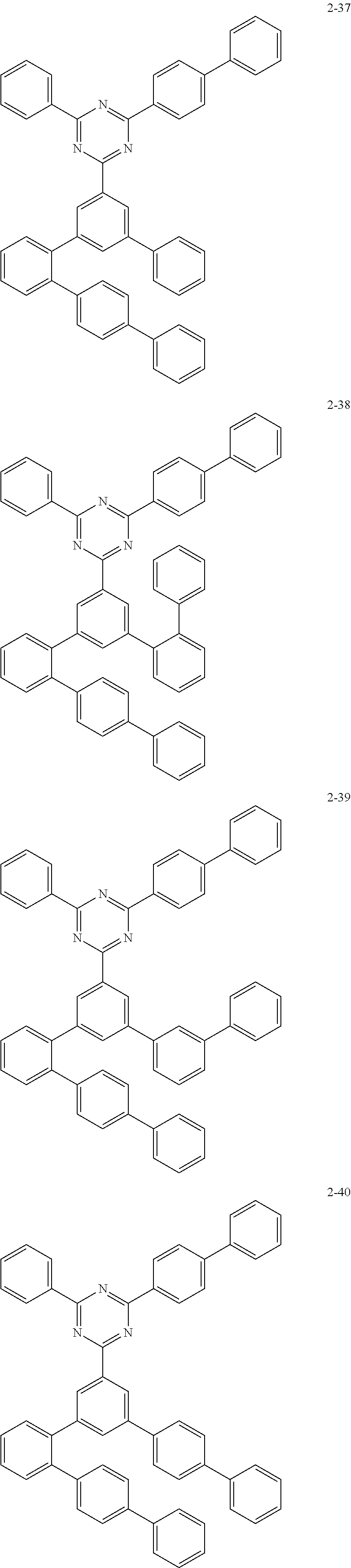 [SMILES: c1ccc(-c2ccc(-c3nc(-c4ccccc4)nc(-c4cc(-c5ccccc5)cc(-c5ccccc5-c5ccc(-c6ccccc6)cc5)c4)n3)cc2)cc1 ; c1ccc(-c2ccc(-c3nc(-c4ccccc4)nc(-c4cc(-c5ccccc5-c5ccccc5)cc(-c5ccccc5-c5ccc(-c6ccccc6)cc5)c4)n3)cc2)cc1 ; c1ccc(-c2ccc(-c3nc(-c4ccccc4)nc(-c4cc(-c5cccc(-c6ccccc6)c5)cc(-c5ccccc5-c5ccc(-c6ccccc6)cc5)c4)n3)cc2)cc1 ; c1ccc(-c2ccc(-c3cc(-c4nc(-c5ccccc5)nc(-c5ccc(-c6ccccc6)cc5)n4)cc(-c4ccccc4-c4ccc(-c5ccccc5)cc4)c3)cc2)cc1]

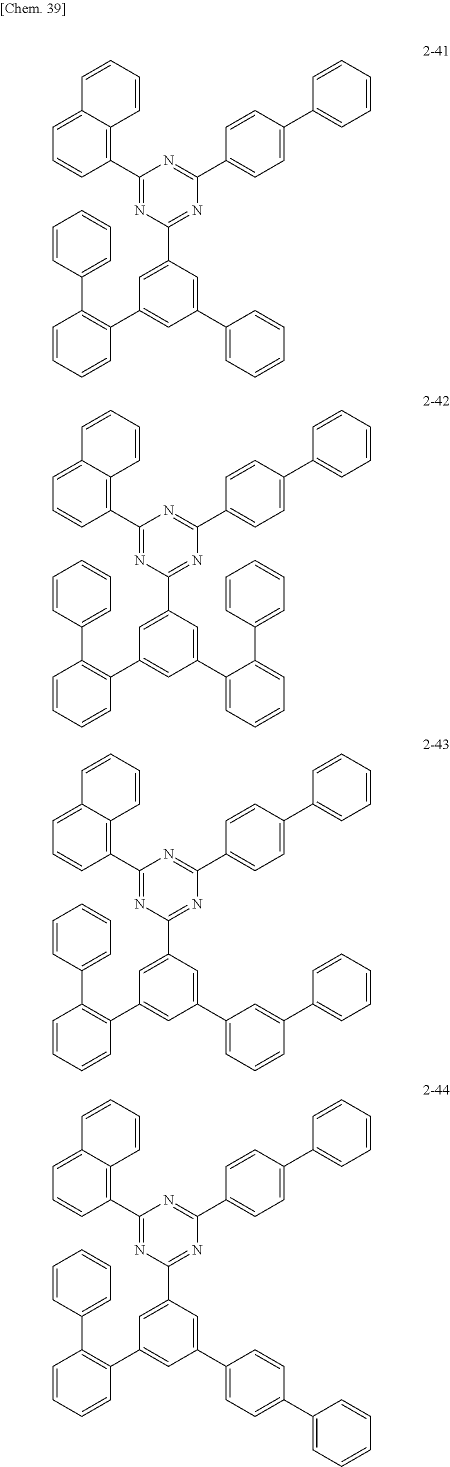

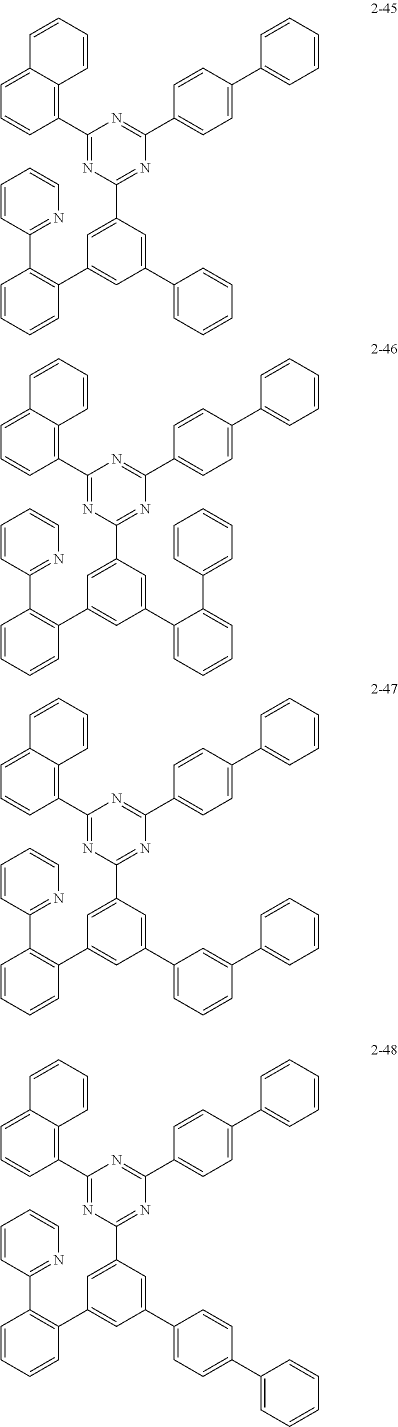

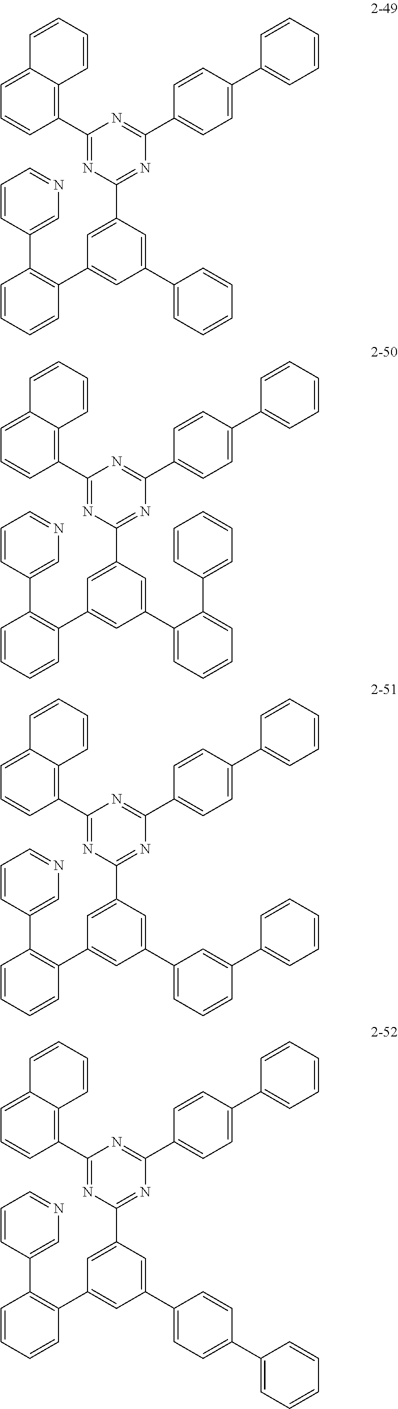

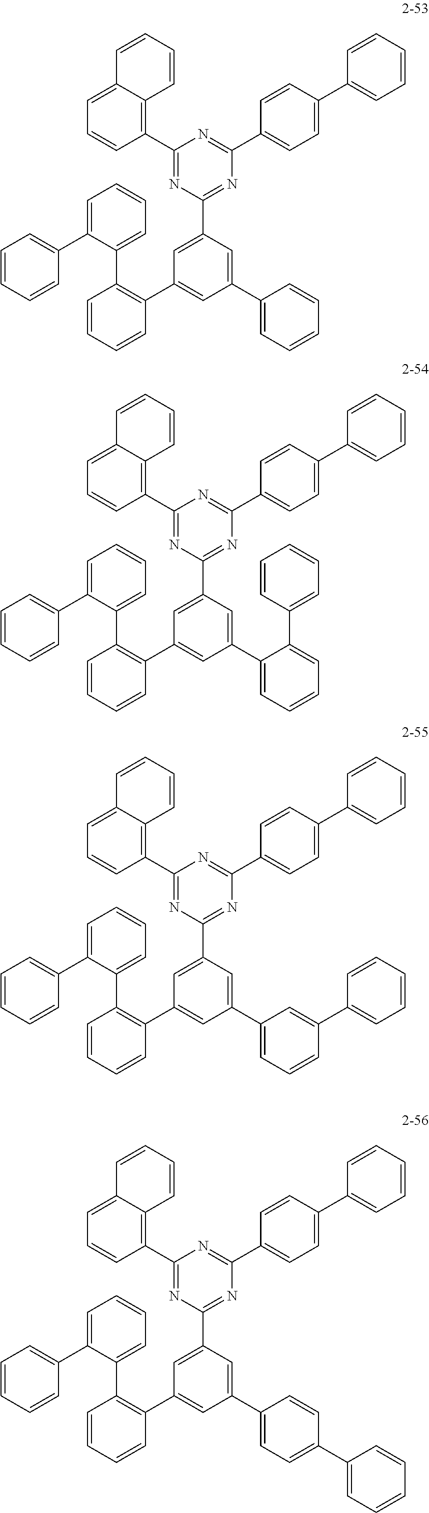 [SMILES: c1ccc(-c2ccc(-c3nc(-c4cc(-c5ccccc5)cc(-c5ccccc5-c5ccccc5-c5ccccc5)c4)nc(-c4cccc5ccccc45)n3)cc2)cc1 ; c1ccc(-c2ccc(-c3nc(-c4cc(-c5ccccc5-c5ccccc5)cc(-c5ccccc5-c5ccccc5-c5ccccc5)c4)nc(-c4cccc5ccccc45)n3)cc2)cc1 ; c1ccc(-c2ccc(-c3nc(-c4cc(-c5cccc(-c6ccccc6)c5)cc(-c5ccccc5-c5ccccc5-c5ccccc5)c4)nc(-c4cccc5ccccc45)n3)cc2)cc1 ; c1ccc(-c2ccc(-c3cc(-c4nc(-c5ccc(-c6ccccc6)cc5)nc(-c5cccc6ccccc56)n4)cc(-c4ccccc4-c4ccccc4-c4ccccc4)c3)cc2)cc1]

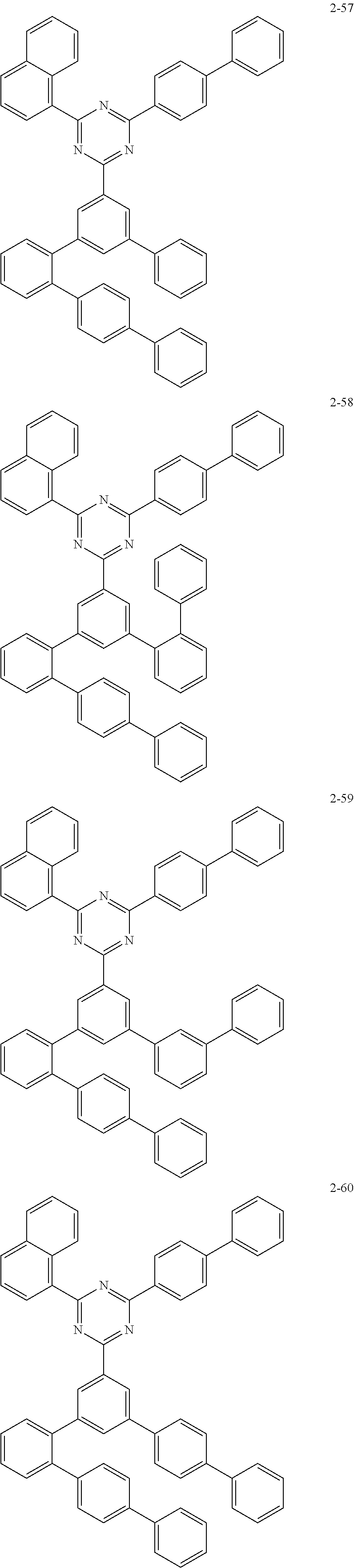 [SMILES: c1ccc(-c2ccc(-c3nc(-c4cc(-c5ccccc5)cc(-c5ccccc5-c5ccc(-c6ccccc6)cc5)c4)nc(-c4cccc5ccccc45)n3)cc2)cc1 ; c1ccc(-c2ccc(-c3nc(-c4cc(-c5ccccc5-c5ccccc5)cc(-c5ccccc5-c5ccc(-c6ccccc6)cc5)c4)nc(-c4cccc5ccccc45)n3)cc2)cc1 ; c1ccc(-c2ccc(-c3nc(-c4cc(-c5cccc(-c6ccccc6)c5)cc(-c5ccccc5-c5ccc(-c6ccccc6)cc5)c4)nc(-c4cccc5ccccc45)n3)cc2)cc1 ; c1ccc(-c2ccc(-c3cc(-c4nc(-c5ccc(-c6ccccc6)cc5)nc(-c5cccc6ccccc56)n4)cc(-c4ccccc4-c4ccc(-c5ccccc5)cc4)c3)cc2)cc1]

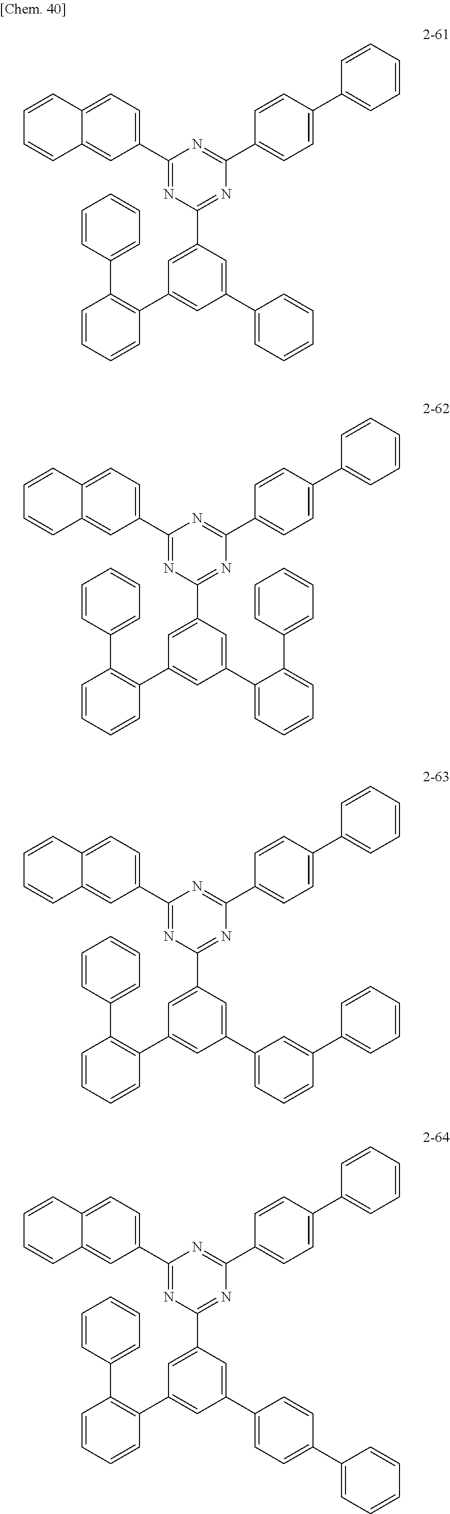 [SMILES: c1ccc(-c2ccc(-c3nc(-c4cc(-c5ccccc5)cc(-c5ccccc5-c5ccccc5)c4)nc(-c4ccc5ccccc5c4)n3)cc2)cc1 ; c1ccc(-c2ccc(-c3nc(-c4cc(-c5ccccc5-c5ccccc5)cc(-c5ccccc5-c5ccccc5)c4)nc(-c4ccc5ccccc5c4)n3)cc2)cc1 ; c1ccc(-c2ccc(-c3nc(-c4cc(-c5cccc(-c6ccccc6)c5)cc(-c5ccccc5-c5ccccc5)c4)nc(-c4ccc5ccccc5c4)n3)cc2)cc1 ; c1ccc(-c2ccc(-c3cc(-c4nc(-c5ccc(-c6ccccc6)cc5)nc(-c5ccc6ccccc6c5)n4)cc(-c4ccccc4-c4ccccc4)c3)cc2)cc1]

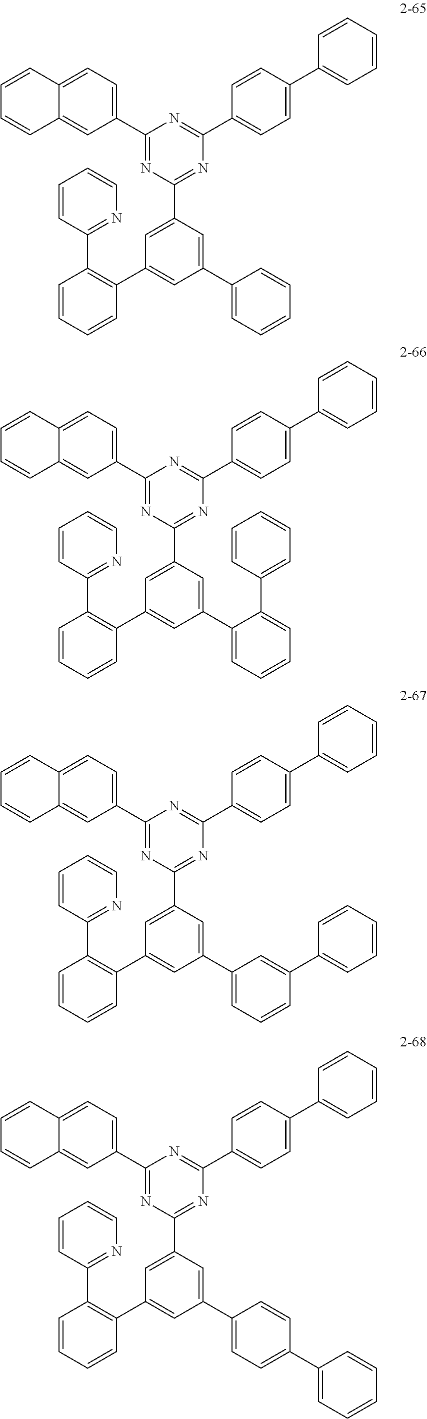 [SMILES: c1ccc(-c2ccc(-c3nc(-c4cc(-c5ccccc5)cc(-c5ccccc5-c5ccccn5)c4)nc(-c4ccc5ccccc5c4)n3)cc2)cc1 ; c1ccc(-c2ccc(-c3nc(-c4cc(-c5ccccc5-c5ccccc5)cc(-c5ccccc5-c5ccccn5)c4)nc(-c4ccc5ccccc5c4)n3)cc2)cc1 ; c1ccc(-c2ccc(-c3nc(-c4cc(-c5cccc(-c6ccccc6)c5)cc(-c5ccccc5-c5ccccn5)c4)nc(-c4ccc5ccccc5c4)n3)cc2)cc1 ; c1ccc(-c2ccc(-c3cc(-c4nc(-c5ccc(-c6ccccc6)cc5)nc(-c5ccc6ccccc6c5)n4)cc(-c4ccccc4-c4ccccn4)c3)cc2)cc1]

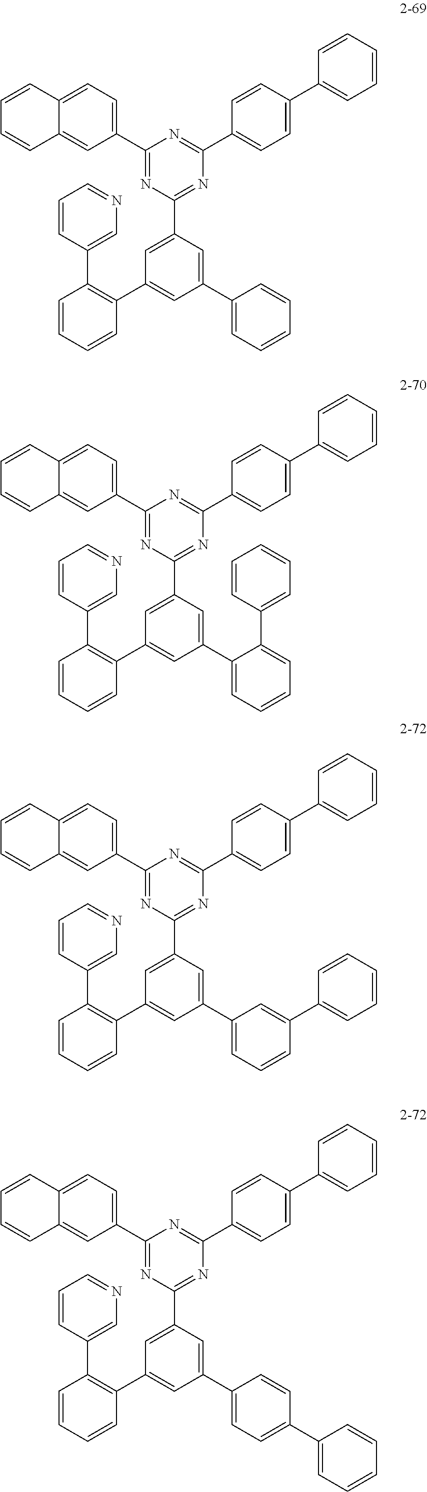 [SMILES: c1ccc(-c2ccc(-c3nc(-c4cc(-c5ccccc5)cc(-c5ccccc5-c5cccnc5)c4)nc(-c4ccc5ccccc5c4)n3)cc2)cc1 ; c1ccc(-c2ccc(-c3nc(-c4cc(-c5ccccc5-c5ccccc5)cc(-c5ccccc5-c5cccnc5)c4)nc(-c4ccc5ccccc5c4)n3)cc2)cc1 ; c1ccc(-c2ccc(-c3nc(-c4cc(-c5cccc(-c6ccccc6)c5)cc(-c5ccccc5-c5cccnc5)c4)nc(-c4ccc5ccccc5c4)n3)cc2)cc1 ; c1ccc(-c2ccc(-c3cc(-c4nc(-c5ccc(-c6ccccc6)cc5)nc(-c5ccc6ccccc6c5)n4)cc(-c4ccccc4-c4cccnc4)c3)cc2)cc1]

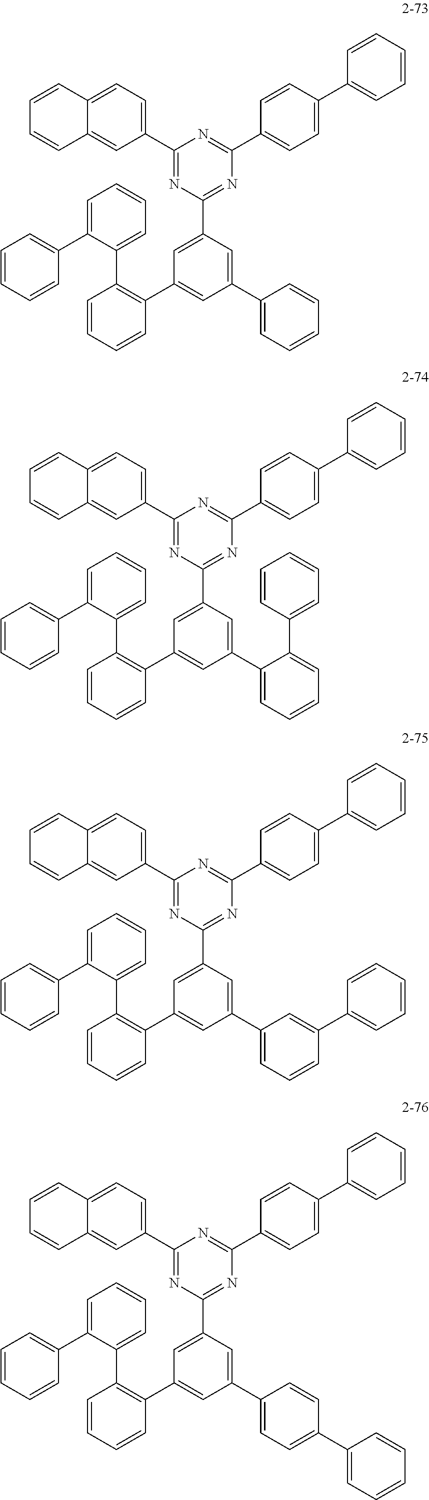 [SMILES: c1ccc(-c2ccc(-c3nc(-c4cc(-c5ccccc5)cc(-c5ccccc5-c5ccccc5-c5ccccc5)c4)nc(-c4ccc5ccccc5c4)n3)cc2)cc1 ; c1ccc(-c2ccc(-c3nc(-c4cc(-c5ccccc5-c5ccccc5)cc(-c5ccccc5-c5ccccc5-c5ccccc5)c4)nc(-c4ccc5ccccc5c4)n3)cc2)cc1 ; c1ccc(-c2ccc(-c3nc(-c4cc(-c5cccc(-c6ccccc6)c5)cc(-c5ccccc5-c5ccccc5-c5ccccc5)c4)nc(-c4ccc5ccccc5c4)n3)cc2)cc1 ; c1ccc(-c2ccc(-c3cc(-c4nc(-c5ccc(-c6ccccc6)cc5)nc(-c5ccc6ccccc6c5)n4)cc(-c4ccccc4-c4ccccc4-c4ccccc4)c3)cc2)cc1]

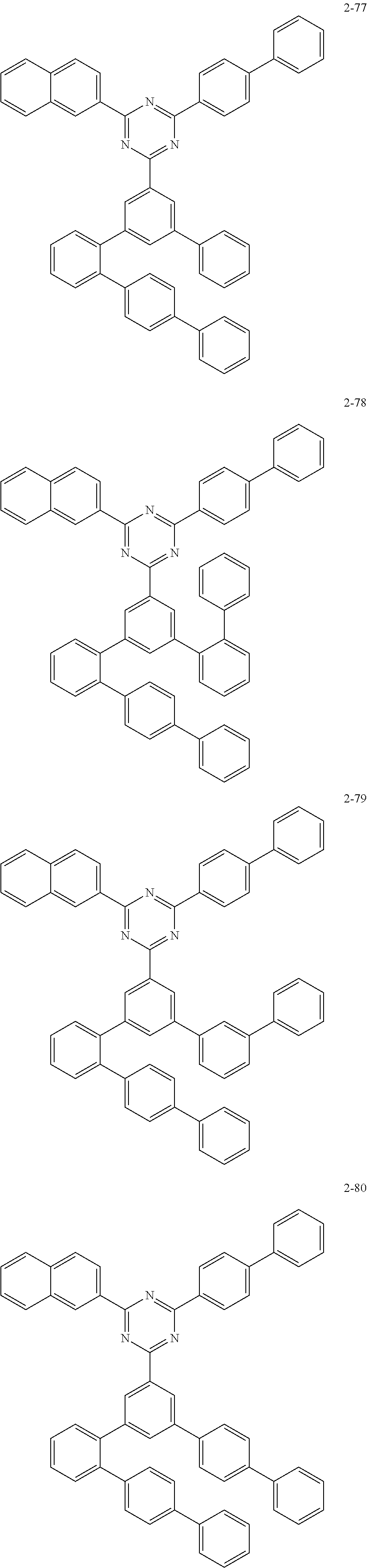 [SMILES: c1ccc(-c2ccc(-c3nc(-c4cc(-c5ccccc5)cc(-c5ccccc5-c5ccc(-c6ccccc6)cc5)c4)nc(-c4ccc5ccccc5c4)n3)cc2)cc1 ; c1ccc(-c2ccc(-c3nc(-c4cc(-c5ccccc5-c5ccccc5)cc(-c5ccccc5-c5ccc(-c6ccccc6)cc5)c4)nc(-c4ccc5ccccc5c4)n3)cc2)cc1 ; c1ccc(-c2ccc(-c3nc(-c4cc(-c5cccc(-c6ccccc6)c5)cc(-c5ccccc5-c5ccc(-c6ccccc6)cc5)c4)nc(-c4ccc5ccccc5c4)n3)cc2)cc1 ; c1ccc(-c2ccc(-c3cc(-c4nc(-c5ccc(-c6ccccc6)cc5)nc(-c5ccc6ccccc6c5)n4)cc(-c4ccccc4-c4ccc(-c5ccccc5)cc4)c3)cc2)cc1]

In particular, the triazine compounds indicated by formulae 2-1, 2-2, 2-4, 2-5, 2-9, 2-22 and 2-24 are preferred, especially because they lead to the coexistence of superior drive voltage characteristics, current efficiency characteristics and efficiency change characteristics of the element.

### [Application of the Triazine Compound (2) to the Organic Electroluminescent Element]

The triazine compound (2) according to an aspect of the present disclosure is used in the electron transport region. The electron transport region preferably forms a multilayer structure, which is composed of multiple layers. More preferably, of these layers, the triazine compound (2) is used in a layer that functions as an electron transport layer or hole-blocking layer as described above, even more preferably in an electron transport layer.

When the triazine compound (2) is used in an electron transport layer, the electron transport layer may be formed solely of the triazine compound (2) or may be formed of a composition with another compound. It is preferred that the layer be formed of a composition with another compound, particularly because it leads to the demonstration of the superior drive voltage of the element. In that case, it is more preferred that the percentage by weight of the triazine compound (2) be from 20% to 80%, even more preferably from 30% to 70%, particularly preferably from 40% to 60%.

The layer formed by a composition of the triazine compound (2) and another compound may be formed by co-deposition, in which each individual one of the triazine compound (2) and the other compound is heated and simultaneously deposited. Alternatively, a mixture of the triazine compound (2) and the other compound may be heated and deposited. From the viewpoint of controlling the percentage of the triazine compound (2) within the resulting layer, it is preferred to form the layer by co-deposition.

The compound that is deposited together with the triazine compound (2) to form a composition is preferably a compound presented as a specific example for materials for an electron transport and a hole-blocking layer as mentioned above or Liq, more preferably Liq.

It should be noted that the triazine compound (2) according to an aspect of the present disclosure can be synthesized by combining known reactions (e.g., the Suzuki-Miyaura cross-coupling reaction) appropriately.

For example, the triazine compound (2) according to an aspect of the present disclosure can be synthesized following a production process indicated by any one of reaction formulae (a) to (c) presented below. The triazine compound (2), however, is not to be construed as limited in any way by these examples.

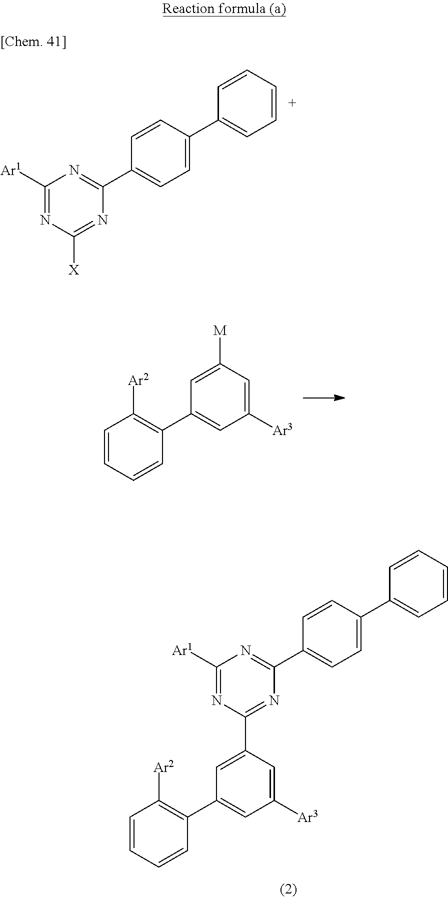

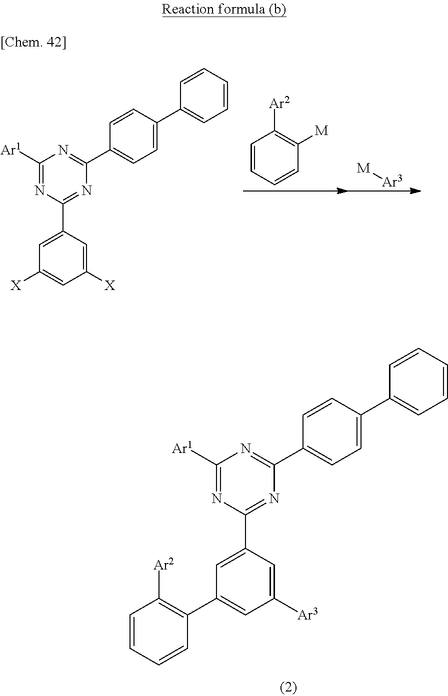

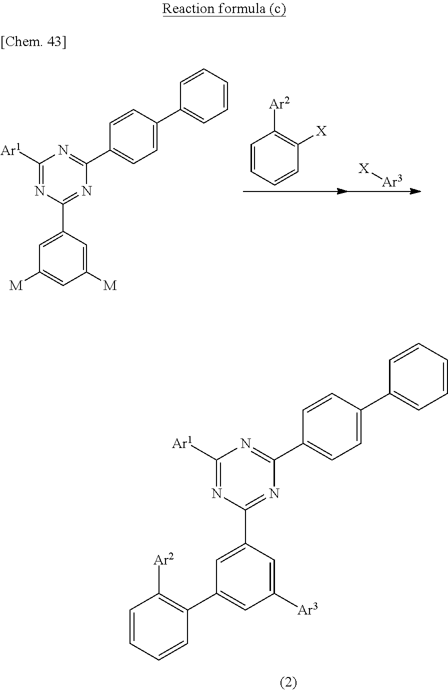

In reaction formulae (a) to (c), Ar¹, Ar² and Ar³ have the same definitions as in formula (1).

X represents a leaving group, independently at each occurrence. Examples of leaving groups are not particularly limited but include a chlorine atom, a bromine atom, an iodine atom and a trifluoromethanesulfonyloxy group. Of these, a bromine atom or a chlorine atom is preferred because it leads to a good reaction yield. In some cases, however, it might be more preferred to use a trifluoromethanesulfonyloxy group because of the availability of raw materials.

M represents ZnR¹, MgR² or Sn(R³)³, which is a metal-containing group; or B(OR⁴)², which is a boron-containing group; independently at each occurrence, with the proviso that R¹ and R² each independently represent a chlorine atom, a bromine atom or an iodine atom; R³ represents a C1 to C4 alkyl group or a phenyl group; R⁴ represents a hydrogen atom, a C1 to C4 alkyl group or a phenyl group; and the two R⁴s in B(OR⁴)² may be the same or different. The two R⁴s, furthermore, may join together to form a ring including the oxygen atoms and the boron atom.

Examples of ZnR¹ and MgR² include ZnCl, ZnBr, ZnI, MgCl, MgBr and MgI.

Examples of Sn(R³)₃ include Sn(Me)₃ and Sn(Bu)₃.

Examples of B(OR⁴)₂ include B(OH)₂, B(OMe)₂, B(OiPr)₂ and B(OBu)₂. Examples of B(OR⁴)₂ when the two R⁴s join together to form a ring including the oxygen atoms and the boron atom are not particularly limited but include the groups represented by (I) to (VI) below. The group represented by (II) is desirable because it leads to a good yield.

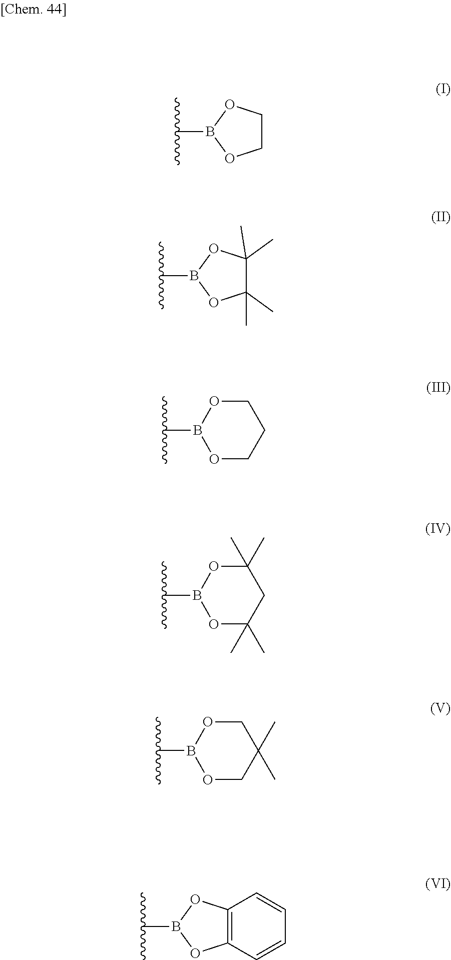

The production processes indicated by reaction formulae (a) to (c) will be described in further detail by citing the production process indicated by reaction formula (b) as an example. The production process indicated by reaction formula (b) represents obtaining the triazine compound (2) by sequentially using organometallic compounds containing Ar² or Ar³ for reaction in the presence of a palladium catalyst. The organometallic compounds containing Ar² or Ar³ used for reaction can be used and allowed to react simultaneously, or the triazine compound (2) can be obtained by once isolating an intermediate product obtained by causing a reaction using the organometallic compound containing Ar² and then causing a reaction using the organometallic compound containing Ar³ in the presence of a palladium catalyst. It is also possible to obtain the triazine compound (2) by using the organometallic compound containing Ar³ for reaction first.

### [Negative Electrode 6]

With the electron transport region 5 in between, a negative electrode 6 opposing the positive electrode 2 is provided. The negative electrode 6 can serve a function as a negative electrode in passing an electric current through the organic electroluminescent element that is an aspect of the present disclosure.

In the case of an organic electroluminescent element in a configuration in which only the emitted light that has passed through the positive electrode 2 is extracted, the negative electrode 6 can be formed from any electrically conductive material.

Examples of materials for the negative electrode 6 include lithium, sodium, sodium-potassium alloys, magnesium, silver, magnesium/copper mixtures, magnesium/silver mixtures, magnesium/aluminum mixtures, magnesium/indium mixtures, aluminum/aluminum oxide (Al₂O₃) mixtures, indium, indium zinc oxide (IZO), lithium/aluminum mixtures and rare earth metals, such as ytterbium.

In the case of an organic electroluminescent element in a configuration in which the light is extracted solely from the negative electrode 6 side, examples include using a transparent electrode material, such as indium zinc oxide (IZO), or shaping a metallic material, such as silver, aluminum or a silver/magnesium mixture, to a thickness that allows the emitted light to pass through sufficiently.

### [Method for Forming Each Layer]

Each layer described above, excluding the electrodes (positive electrode and negative electrode), can be formed by shaping the material for the layer (optionally with a solvent and materials such as a binder resin) into a thin film by a known method, such as vacuum deposition, spin coating, casting or the LB (Langmuir-Blodgett method) method.

There is no specific restriction on the thickness of each layer formed in such a manner; the thickness can be selected as appropriate according to the situation. Usually, however, the thickness is in the range of 1 nm to 5 μm.

The positive electrode 2 and the negative electrode 6 can be formed by shaping an electrode material into a thin film by a method such as deposition or sputtering. A pattern may be formed using a mask in the desired shape during deposition or sputtering, or a thin film may be formed first, for example by deposition or sputtering, and then a pattern in the desired shape may be formed by photolithography.

The thickness of the positive electrode 2 and the negative electrode 6 is preferably 1 μm or less, more preferably 5 nm or more and 200 nm or less. In general, when a material with low transmittance is used for the electrode on the side from which the emitted light is extracted, sufficient transmittance can be ensured by forming the film to 30 nm or thinner, allowing it to act as a transparent electrode.

The organic electroluminescent element according to an aspect of the present disclosure may be used as a type of lamp, such as one for illumination or an exposure light source, or may be used as a projection device of a type that projects images or a display device of a type that allows the user to view still images or moving pictures directly (display). The driving scheme when it is used as a display device for movie playback can be either the simple matrix (passive matrix) scheme or the active matrix scheme. By using two or more types of organic electroluminescent elements according to this aspect with different colors of emitted light, furthermore, a full-color display device can be fabricated.

### EXAMPLES

The present disclosure will now be described in further detail based on examples. The present disclosure, however, is not to be construed as limited in any way by these examples.

The measurement of the 1H-NMR spectra was performed using Gemini 200 (manufactured by Varian, Inc.) or Bruker ASCEND 400 (400 MHZ; manufactured by BRUKER CORPORATION).

The emission characteristics of the organic electroluminescent elements were evaluated by applying a DC current to the fabricated elements at room temperature and using a luminance meter (product name, BM-9; manufactured by Topcon Technohouse Corporation).

### Synthesis Example—1 (Synthesis of Compound 2-1)

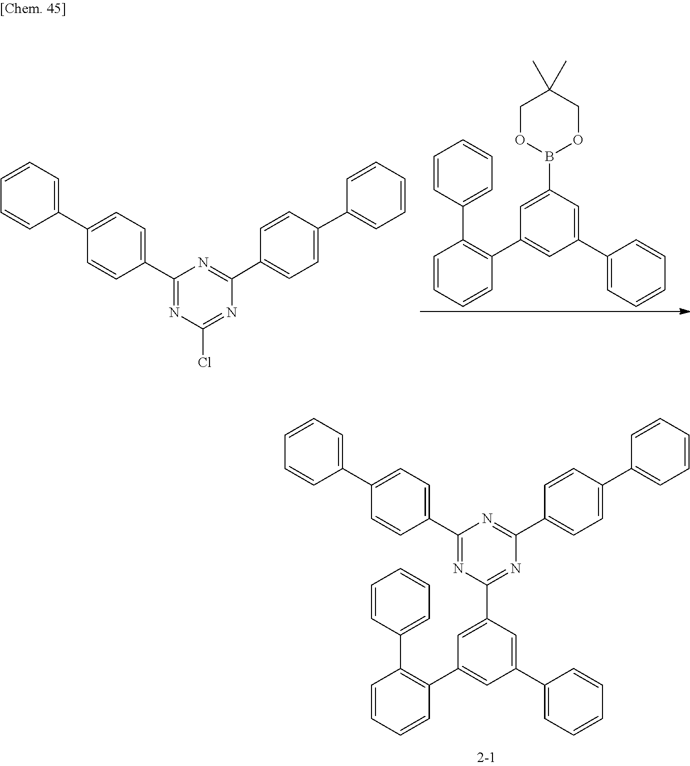

In an argon atmosphere, 2-chloro-4,6-bis(biphenyl-4-yl)-1,3,5-triazine (11.0 g, 26.2 mmol), 5′-(5,5-dimethyl-1,3,2-dioxaborinan-2-yl)-1,1′:3′,1″:2″,1″′-quaterphenyl (12.1 g, 28.8 mmol), tetrakis(triphenylphosphine) palladium (0.91 g, 0.79 mmol) and a 2 M-aqueous solution of potassium phosphate (39.3 mL) were suspended in THE (349 mL), and the resulting mixture was refluxed for 3 hours. After natural cooling, water and methanol were added to the reaction mixture, and the solid was collected by filtration and washed with water and methanol to give 2-(1,1′:3′,1″:2″,1′″-quaterphenyl-5′-yl)-4,6-bis(biphenyl-4-yl)-1,3,5-triazine (2-1) (actual yield, 17.9 g; percent yield, 99%).

### Synthesis Example—2 (Synthesis of Compound 2-2)

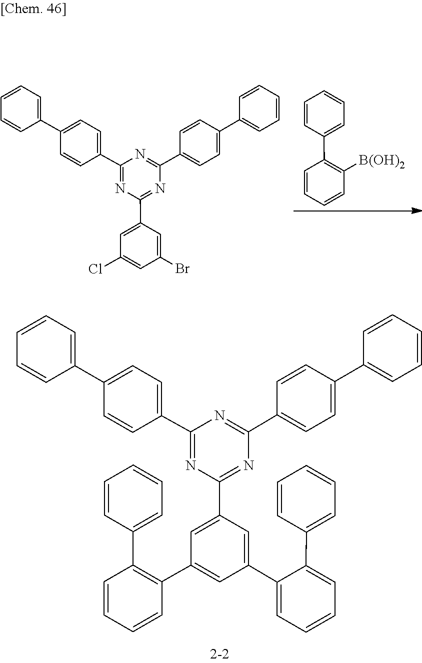

In an argon atmosphere, 2-(3-bromo-5-chlorophenyl)-4,6-bis(biphenyl-4-yl)-1,3,5-triazine (5.75 g, 10.0 mmol), 2-biphenylboronic acid (4.75 g, 24.0 mmol), a 3 M-aqueous solution of potassium carbonate (16 mL), palladium acetate (22.4 mg, 0.1 mmol) and 2-dicyclohexylphosphino-2′,4′,6′-triisopropylbiphenyl (XPhos, 95.3 mg, 0.2 mmol) were suspended in THE (100 mL), and the resulting mixture was refluxed for 96 hours. After natural cooling, water and methanol were added to the reaction mixture, and the solid was collected by filtration and washed with water and methanol to give 2-(1,1′:2′,1″:3″,1′″:2′″,1″″-quinquephenyl-5″-yl)-4,6-bis(biphenyl-4-yl)-1,3,5-triazine (2-2) (actual yield, 7.18 g; percent yield, 94%).

### Synthesis Example—3 (Synthesis of Compound 2-22)

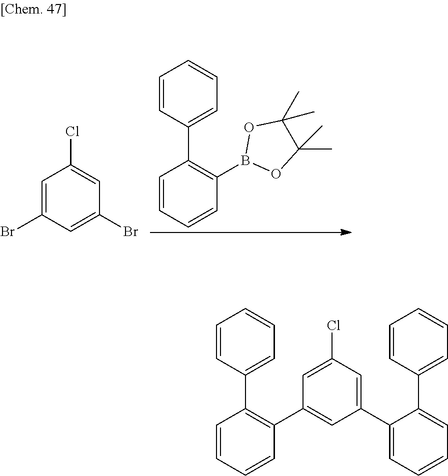

In an argon atmosphere, 1,3-dibromo-5-chlorobenzene (20.0 g, 74.0 mmol), 2-(4,4,5,5-tetramethyl-1,3,2-dioxaborolan-2-yl)-biphenyl (42.5 g, 151.7 mmol), a 3 M-aqueous solution of potassium carbonate (110 mL) and tetrakis triphenylphosphine palladium (2.56 g, 2.22 mmol) were suspended in THE (245 mL), and the resulting mixture was refluxed for 122 hours. After natural cooling, toluene was added to the reaction mixture, and the organic layer was extracted by separation. The organic layer obtained was distilled away under reduced pressure, and the resulting solid was recrystallized in toluene to give 5″-chloro-(1,1′:2′,1″:3″,1′″:2′″,1″″-quinquephenyl (actual yield, 29.7 g; percent yield, 96%).

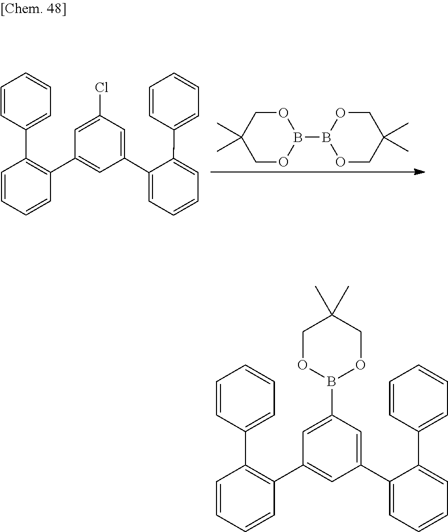

In an argon atmosphere, the 5″-chloro-(1,1′:2′,1″:3″,1′″:2′″,1″″-quinquephenyl (29.7 g, 71.2 mmol), bis(neopentylglycolato)diboron (17.7 g, 78.3 mmol), potassium acetate (0.16 g, 0.71 mmol), palladium acetate (20.9 g, 213.5 mmol) and 2-dicyclohexylphosphino-2′,4′,6′-triisopropylbiphenyl (XPhos, 0.67 mg, 1.42 mmol) were suspended in THE (356 mL), and the resulting mixture was refluxed for 27 hours. After that, the solvent was distilled under reduced pressure, and methanol was added to the resulting residual. The resulting solid was collected by filtration and dried under reduced pressure to give the desired 5″-(5,5-dimethyl-1,3,2-dioxaborinan-2-yl)-1,1′:2′,1″:3″,1′″:2″′,1″″-quinquephenyl (actual yield, 14.7 g; percent yield, 42%).

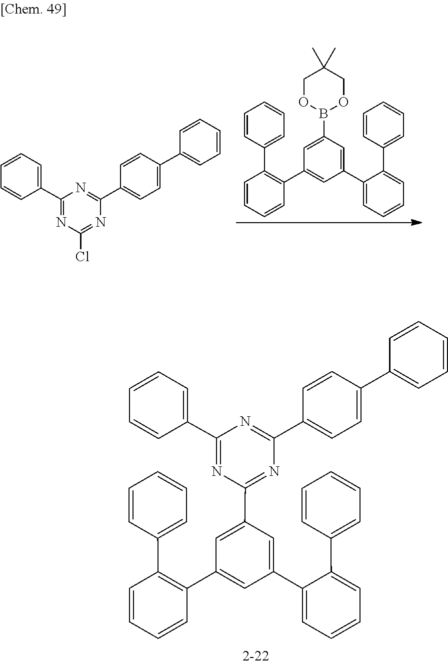

In an argon atmosphere, 2-chloro-4-(biphenyl-4-yl)-6-phenyl-1,3,5-triazine (5.56 g, 16.2 mmol), the 5″-(5,5-dimethyl-1,3,2-dioxaborinan-2-yl)-1,1′:2′,1″:3″,1′″:2″′,1″″-quinquephenyl (24.0 g, 48.5 mmol), tetrakis(triphenylphosphine) palladium (0.56 g, 0.49 mmol) and a 2 M-aqueous solution of potassium phosphate (24.0 mL) were suspended in THE (324 mL), and the resulting mixture was refluxed for 23 hours. After natural cooling, water and methanol were added to the reaction mixture, and the solid was collected by filtration and washed with water and methanol. The resulting solid was recrystallized in toluene to give 2-(1,1′:2′,1″:3″,1′″:2′″,1′″-quinquephenyl-5″-yl)-4-(biphenyl-4-yl)-6-phenyl-1,3,5-triazine (2-22) (actual yield, 11.1 g; percent yield, 99%).

### Synthesis Example—4 (Synthesis of Compound 2-24)

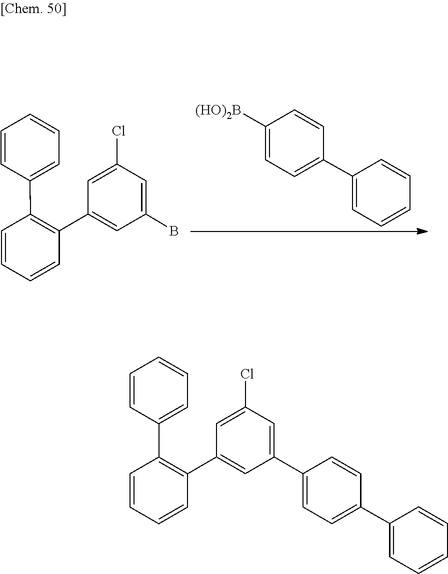

In an argon atmosphere, 3-bromo-5-chloro-1,1′:2′,1″-terphenyl (g, mmol), 4-biphenylboronic acid (g, mmol), tetrakis triphenylphosphine palladium (g, mmol) and a 2 M-aqueous solution of potassium phosphate (58.9 mL) were suspended in THE (393 mL), and the resulting mixture was refluxed for 21 hours. After natural cooling, the organic layer was extracted by separation, and the organic layer obtained was distilled away under reduced pressure. Ethanol was added to the resulting residual, and the resulting solid was collected by filtration to give the desired 5″-chloro-1,1′:2′,1″:3″,1′″:4″′,1″″-quinquephenyl (actual yield, 24 g; percent yield, 100%)

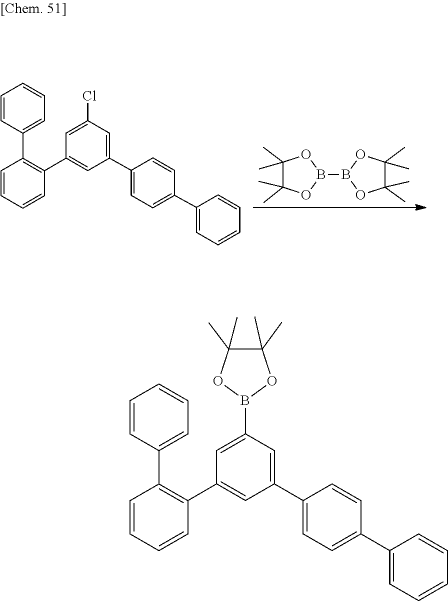

In an argon atmosphere, the 5″-chloro-1,1′:2′,1″:3″,1′″:4′″,1″″-quinquephenyl (23.1 g, 55.5 mmol), bis(pinacolato)diboron (16.9 g, 66.5 mmol), potassium acetate (16.3 g, 166.4 mmol), palladium acetate (0.25 g, 1.11 mmol) and 2-dicyclohexylphosphino-2′,4′,6′-triisopropylbiphenyl (XPhos, 1.06 mg, 2.22 mmol) were suspended in THE (555 mL), and the resulting mixture was refluxed for 19 hours. After that, the solvent was distilled under reduced pressure, and methanol was added to the resulting residual. The resulting solid was collected by filtration and dried under reduced pressure to give the desired 5″-(4,4,5,5-tetramethyl-1,3,2-dioxaborolan-2-yl)-1,1′:2′,1″:3″,1′″:4″′,1″″-quinquephenyl (actual yield, 22.0 g; percent yield, 78%)

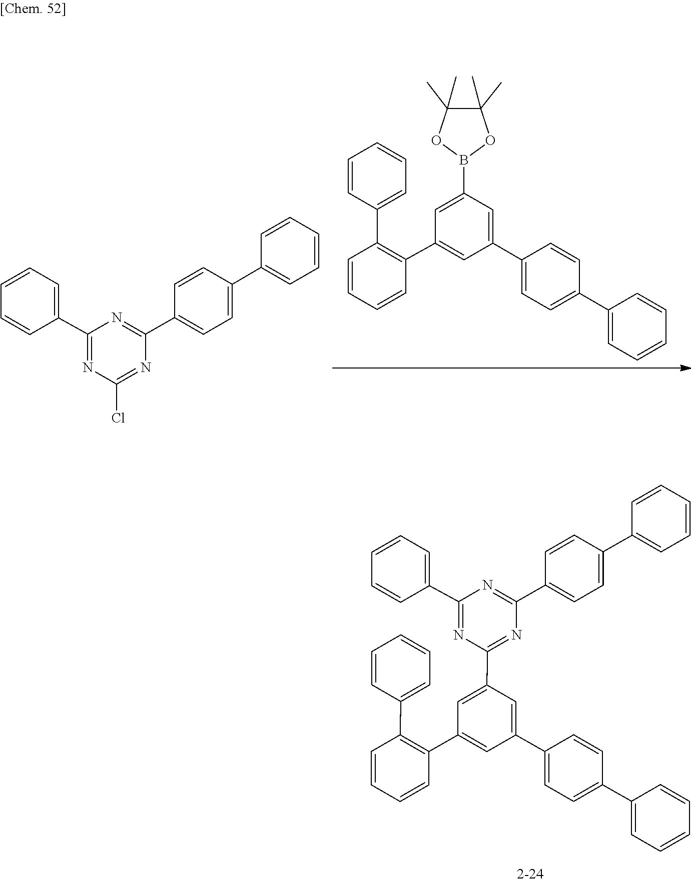

In an argon atmosphere, 2-chloro-4-(biphenyl-4-yl)-6-phenyl-1,3,5-triazine (13.5 g, 39.3 mmol), the 5″-(4,4,5,5-tetramethyl-1,3,2-dioxaborolan-2-yl)-1,1′:2′,1″:3″,1′″:4′″,1″″-quinquephenyl (22.0 g, 43.2 mmol), tetrakis(triphenylphosphine) palladium (0.91 g, 0.79 mmol) and a 2 M-aqueous solution of potassium phosphate (58.9 mL) were suspended in THE (393 mL), and the resulting mixture was refluxed for 21 hours. After natural cooling, water and methanol were added to the reaction mixture, and the solid was collected by filtration and washed with water and methanol. The resulting solid was recrystallized in toluene to give 2-(1,1′:2′,1″:3″,1′″:4′″,1″″-quinquephenyl-5″-yl)-4-(biphenyl-4-yl)-6-phenyl-1,3,5-triazine (2-24) (actual yield, 21.1 g; percent yield, 85%).

As for triazine compound 2-4, furthermore, it was synthesized by means similar to the production process presented in Synthesis Example—1.

The structural formulae of the compounds used for the fabrication and performance evaluation of the organic electroluminescent elements and their abbreviations are presented below.

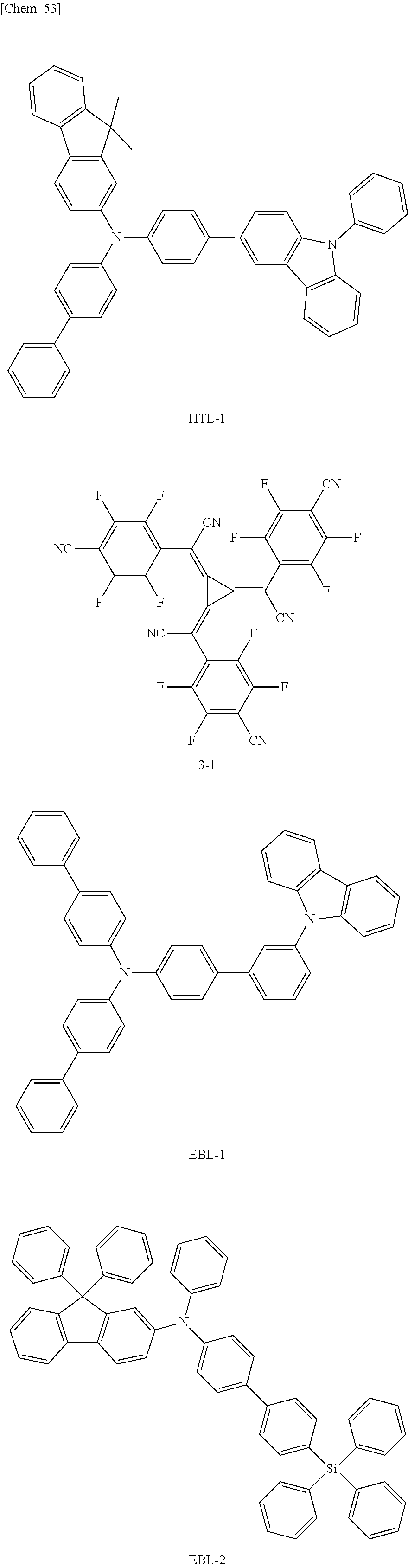 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1]

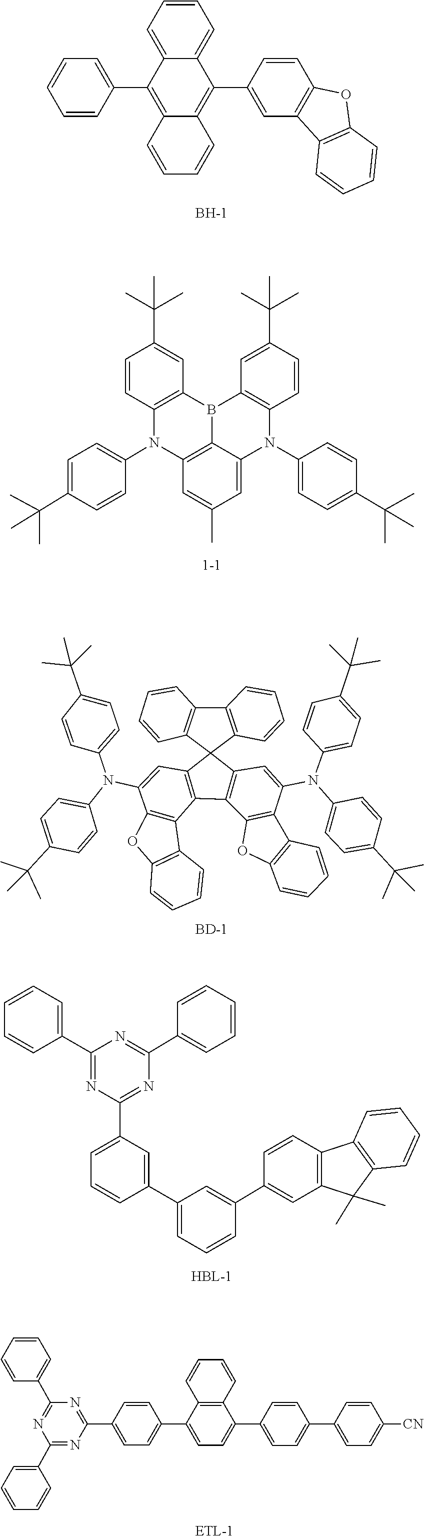

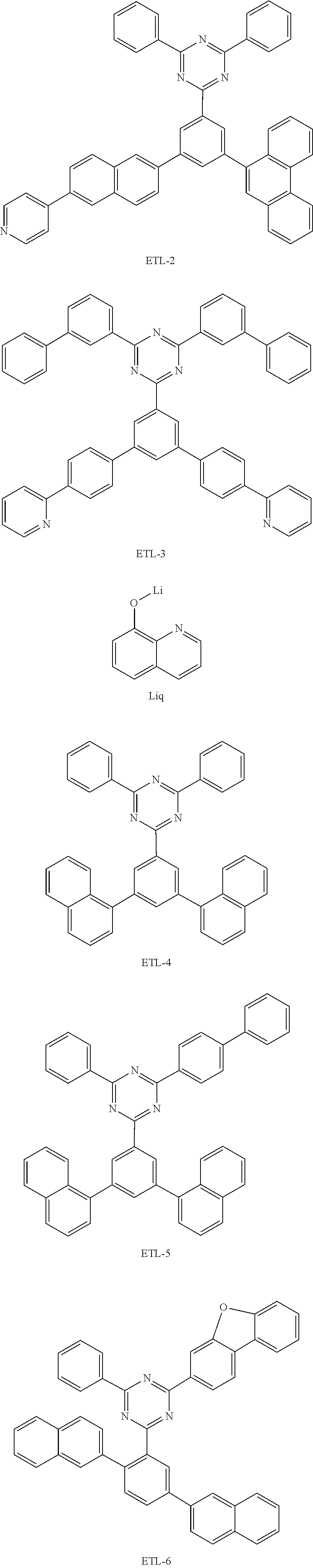

### Example—1 (see FIG. 1)

### (Preparation of a Substrate 1 and a Positive Electrode 2)

As a substrate having a positive electrode on its surface, a glass substrate with an indium tin oxide (ITO) transparent electrode was prepared, on which 2-mm width ITO films (thickness, 110 nm) were arranged in a stripe pattern. Then this substrate was washed with isopropyl alcohol, and surface treatment was performed by ozone ultraviolet cleaning thereafter.

### (Preparation for Vacuum Deposition)

On the substrate that had undergone surface treatment after washing, vacuum deposition of each layer was performed by the vacuum deposition technique, through which the layers were stacked and formed.

First, the glass substrate was introduced into a vacuum deposition chamber, and the pressure was reduced to 1.0×10⁻⁴ Pa. Then the layers were produced one by one in the following order, according to film formation conditions for each layer.

### (Production of a First Hole Transport Layer 31)

A first hole transport layer 31 was produced by shaping HTL-1 purified by sublimation and compound 3-1, which is an aspect of the present disclosure, into a 10-nm film at a rate of 0.15 nm/second.

### (Production of a Second Hole Transport Layer 32)

A second hole transport layer 32 was produced by shaping HTL-1 purified by sublimation into an 85-nm film at a rate of 0.15 nm/second.

### (Production of a Third Hole Transport Layer 33)

A third hole transport layer 33 was produced by shaping EBL-1 purified by sublimation into a 5-nm film at a rate of 0.15 nm/second.

### (Production of a Light-Emitting Layer 4)

A light-emitting layer 4 was produced by shaping BH-1 purified by sublimation and compound 1-1, which is an aspect of the present disclosure, in proportions of 95:5 (ratio by mass) into a 20-nm film. The film formation rate was 0.18 nm/second.

### (Production of a First Electron Transport Layer 51)

A first electron transport layer 51 was produced by shaping HBL-1 purified by sublimation into a 6-nm film at a rate of 0.05 nm/second.

### (Production of a Second Electron Transport Layer 52)

A second electron transport layer 52 was produced by shaping compound 2-1 and Liq in proportions of 50:50 (ratio by mass) into a 25-nm film. The film formation rate was 0.15 nm/second.

### (Production of a Third Electron Transport Layer 53)

A third electron transport layer 53 was produced by shaping ytterbium into a 2-nm film. The film formation rate was 0.01 nm/second.

### (Production of a Negative Electrode 6)

Finally, a negative electrode 6 was formed as a film, with a metal mask placed orthogonally to the ITO stripes on the substrate. The negative electrode was formed by shaping silver/magnesium (ratio by mass, 9/1) and silver into 12-nm and 90-nm films, respectively, in this order; the electrode had a two-layer structure. The film formation rate for silver/magnesium was 0.5 nm/second, and the film formation rate for silver was a film formation rate of 0.2 nm/second.

Through the foregoing, an organic electroluminescent element 100 with an emission area of 4 mm² as illustrated in FIG. 1 was fabricated. It should be noted that the thickness of each layer was measured using a stylus profiler (DEKTAK, manufactured by Bruker Corporation).

This element, furthermore, was sealed inside a nitrogen-atmosphere glove box with oxygen and water concentrations of 1 ppm or less. The sealing was performed by joining a sealing cap made of glass and the substrate with films formed thereon (element) using a bisphenol F epoxy resin (manufactured by Nagase ChemteX Corporation).

A DC current was applied to the organic electroluminescent element fabricated as described above, and the emission characteristics were evaluated using a luminance meter (product name, BM-9; manufactured by Topcon Technohouse Corporation). As the emission characteristics, the drive voltage (V) and current efficiency (cd/A) when a luminance of 1000 cd/m² was indicated under an applied voltage were measured. It should be noted that the drive voltage and the current efficiency are relative values, assuming that the result in Comparative Example—1 below is a reference value (100). Additionally, the efficiency change was calculated based on the following definition.

$$Efficiency change = ( current efficiency at 1000 cd / {m}^{2} ) / ( current efficiency at 10 cd / {m}^{2} )$$

The closer the value calculated according to this formula is to 1.0, the smaller the change in efficiency within a broad luminance range.

In addition, the element fabricated as described above was operated continuously for 50 hours from a luminance of 1000 cd/m², and then the current efficiency after continuous operation (cd/A) when a luminance of 1000 cd/m² was indicated under a voltage applied once again was measured. The efficiency change after continuous operation, furthermore, was calculated based on the following definition.

$$Efficiency change after continuous operation = ( current efficiency after continuous operation at 1000 cd / {m}^{2} ) / ( current efficiency at 1000 cd / {m}^{2} )$$

The closer the value calculated according to this formula is to 1.0, the smaller the change in efficiency between before and after continuous operation.

The measurement results obtained are presented in Table 1.

### Example—2

An organic electroluminescent element was fabricated and evaluated by the same method as in Example—1, except that compound 2-2 was used instead of compound 2-1 in Element Example—1. The measurement results obtained are presented in Table 1.

### Example—3

An organic electroluminescent element was fabricated and evaluated by the same method as in Example—1, except that compound 2-22 was used instead of compound 2-1 in Element Example—1. The measurement results obtained are presented in Table 1.

### Example—4

An organic electroluminescent element was fabricated and evaluated by the same method as in Example—1, except that compound 2-24 was used instead of compound 2-1 in Element Example—1. The measurement results obtained are presented in Table 1.

### Example—5

An organic electroluminescent element was fabricated and evaluated by the same method as in Example—1, except that compound 2-4 was used instead of compound 2-1 in Element Example—1. The measurement results obtained are presented in Table 1.

### Comparative Example—1

An organic electroluminescent element was fabricated and evaluated by the same method as in Example—1, except that ETL-1 was used instead of compound 2-1 in Element Example—1. The measurement results obtained are presented in Table 1.

### Comparative Example—3

An organic electroluminescent element was fabricated and evaluated by the same method as in Example—1, except that ETL-2 was used instead of compound 2-1 in Element Example—1. The measurement results obtained are presented in Table 1.

### Comparative Example—4

An organic electroluminescent element was fabricated and evaluated by the same method as in Example—1, except that ETL-3 was used instead of compound 2-1 in Element Example—1. The measurement results obtained are presented in Table 1.

### Comparative Example—5

An organic electroluminescent element was fabricated and evaluated by the same method as in Example—1, except that ETL-4 was used instead of compound 2-1 in Element Example—1. The measurement results obtained are presented in Table 1.

### Comparative Example—6

An organic electroluminescent element was fabricated and evaluated by the same method as in Example—1, except that ETL-5 was used instead of compound 2-1 in Element Example—1. The measurement results obtained are presented in Table 1.

### Comparative Example—7

An organic electroluminescent element was fabricated and evaluated by the same method as in Example—1, except that ETL-6 was used instead of compound 2-1 in Element Example—1. The measurement results obtained are presented in Table 1.

TABLE 1

| | | Second | | | | Efficiency |
|---|---|---|---|---|---|---|
| | Hole- | electron | | | | change after |
| | generating | transport | Drive | Current | Efficiency | continuous |
| | material | layer | voltage | efficiency | change | operation |
| Example-1 | 3-1 | 2-1 | 96 | 113 | 1.05 | 0.94 |
| Example-2 | 3-1 | 2-2 | 96 | 110 | 1.01 | 0.92 |
| Example-3 | 3-1 | 2-22 | 96 | 111 | 0.99 | 0.91 |
| Example-4 | 3-1 | 2-24 | 96 | 110 | 1.06 | 0.92 |
| Example-5 | 3-1 | 2-4 | 95 | 111 | 1.04 | 0.96 |
| Comparative | 3-1 | ETL-1 | 100 | 100 | 1.25 | 0.64 |
| Example-1 | | | | | | |
| Comparative | 3-1 | ETL-2 | 105 | 99 | 1.18 | 0.58 |
| Example-3 | | | | | | |
| Comparative | 3-1 | ETL-3 | 104 | 96 | 1.22 | 0.50 |
| Example-4 | | | | | | |
| Comparative | 3-1 | ETL-4 | 104 | 97 | 1.29 | 0.44 |
| Example-5 | | | | | | |
| Comparative | 3-1 | ETL-5 | 103 | 98 | 1.27 | 0.62 |
| Example-6 | | | | | | |
| Comparative | 3-1 | ETL-6 | 104 | 99 | 1.22 | 0.52 |
| Example-7 | | | | | | |

### Example—6

An organic electroluminescent element was fabricated and evaluated by the same method as in Element Example—1, except that EBL-2 was used instead of EBL-1, BD-1 was used instead of compound 1-1, compound 2-4 was used instead of compound 2-1, and Liq was used instead of ytterbium in Example—1. The measurement results obtained are presented in Table 2.

### Example—7

An organic electroluminescent element was fabricated and evaluated by the same method as in Element Example—1, except that 2-24 was used instead of compound 2-4 in Example—6. The results obtained are presented in Table 2.

### Comparative Example—2

An organic electroluminescent element was fabricated and evaluated by the same method as in Element Example—1, except that ETL-1 was used instead of compound 2-4 in Example—6. The results obtained are presented in Table 2.

### Comparative Example—8

An organic electroluminescent element was fabricated and evaluated by the same method as in Element Example—1, except that ETL-2 was used instead of compound 2-4 in Example—6. The results obtained are presented in Table 2.

### Comparative Example—9

An organic electroluminescent element was fabricated and evaluated by the same method as in Element Example—1, except that ETL-3 was used instead of compound 2-4 in Example—6. The results obtained are presented in Table 2.

### Comparative Example—10

An organic electroluminescent element was fabricated and evaluated by the same method as in Element Example—1, except that HIL-1 was used instead of BD-1 in Example—6. The results obtained are presented in Table 2.

### Comparative Example—11

An organic electroluminescent element was fabricated and evaluated by the same method as in Element Example—1, except that HIL-1 was used instead of BD-1, and 2-24 was used instead of compound 2-4 in Example—6. The results obtained are presented in Table 2.

TABLE 2

| | | Second | | | | Efficiency |
|---|---|---|---|---|---|---|
| | Hole- | electron | | | | change after |
| | generating | transport | Drive | Current | Efficiency | continuous |
| | material | layer | voltage | efficiency | change | operation |
| Example-6 | 3-1 | 2-4 | 99 | 105 | 1.08 | 0.85 |
| Example-7 | 3-1 | 2-24 | 97 | 104 | 1.07 | 0.84 |
| Comparative | 3-1 | ETL-1 | 100 | 100 | 1.13 | 0.64 |
| Example-2 | | | | | | |
| Comparative | 3-1 | ETL-2 | 97 | 101 | 1.15 | 0.55 |
| Example-8 | | | | | | |
| Comparative | 3-1 | ETL-3 | 104 | 103 | 1.09 | 0.51 |
| Example-9 | | | | | | |
| Comparative | HIL-1 | 2-4 | 110 | 90 | 1.41 | 0.48 |
| Example-10 | | | | | | |
| Example-11 | HIL-1 | 2-24 | 112 | 92 | 1.38 | 0.46 |

According to Tables 1 and 2, a configuration in which an electron transport region according to an aspect of the present disclosure contains a triazine compound represented by formula (2) above and in which a light-emitting layer according to an aspect of the present disclosure contains a boron compound represented by formula (1) above, or/and a hole transport region contains a radialene compound represented by formula (3) above can provide an organic electroluminescent element that achieves not only superior drive voltage characteristics and current efficiency characteristics compared with known configurations, but also a small efficiency change and a small efficiency change after continuous operation.

While the present invention has been described in detail and with reference to specific embodiments, it is apparent to one skilled in the art that various changes and modifications can be made without departing from the nature and scope of the present invention.

It should be noted that the entire contents of the description, claims, drawings and abstract of Japanese Patent Application No. 2022-106807 and Japanese Patent Application No. 2022-106810, filed on Jul. 1, 2022, and Japanese Patent Application No. 2023-088821 and Japanese Patent Application No. 2023-088916, filed on May 30, 2023, are incorporated herein by reference as a disclosure of the description of the present invention.

### REFERENCE SIGNS LIST

1 Substrate
2 Positive electrode
3 Hole transport region
31 First hole transport layer
32 Second hole transport layer
33 Third hole transport layer
4 Light-emitting layer
5 Electron transport region
51 First electron transport layer
52 Second electron transport layer
53 Third electron transport layer
6 Negative electrode
100 Organic electroluminescent element

1. An organic electroluminescent element comprising:
a positive electrode;
a negative electrode opposite the positive electrode;
a light-emitting layer disposed between the positive electrode and the negative electrode; and
a hole transport region disposed between the positive electrode and the light-emitting layer, wherein:
the organic electroluminescent element includes an electron transport region disposed between the light-emitting layer and the negative electrode;
the electron transport region contains a triazine compound represented by formula (2); and
the organic electroluminescent element satisfies at least one or more of condition (i) or (ii) below.
(i) The light-emitting layer contains a boron compound represented by formula (1).
(ii) The hole transport region contains a radialene compound represented by formula (3). 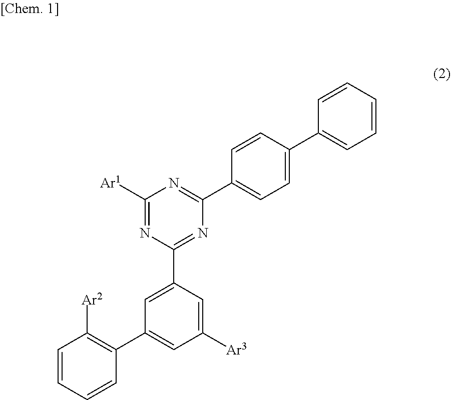
In formula (2),
Ar¹ represents a phenyl group, naphthyl group or biphenylyl group optionally substituted with a C1 to C6 alkyl group, and
Ar² represents a phenyl group, pyridyl group, phenylpyridyl group, pyridylphenyl group or biphenylyl group optionally substituted with a C1 to C4 alkyl group.
Ar³ represents a phenyl group or biphenylyl group optionally substituted with a C1 to C4 alkyl group. 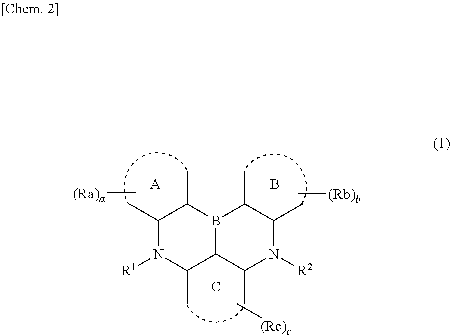
In formula (1),
ring A, ring B and ring C each independently represent
an aromatic hydrocarbon ring in which the number of ring-constituting atoms is from 6 to 20 or
an aromatic heterocycle in which the number of ring-constituting atoms is from 5 to 20,
each optionally having a substituent;
Ra, Rb and Rc each independently represent
deuterium,
a cyano group,
a C1 to C20 alkyl group,
a substituted or unsubstituted amino group,
an aromatic hydrocarbon group in which the number of ring-constituting atoms is from 6 to 20 or
a heteroaromatic group in which the number of ring-constituting atoms is from 5 to 20;
each of a, b and c is independently an integer of 0 to 4,
ring A and ring B are optionally linked together via an oxygen atom, a sulfur atom, N—R′, Ra or Rb,
R¹, R² and R′ each independently represent
a C1 to C20 alkyl group,
an aromatic hydrocarbon group in which the number of ring-constituting atoms is from 6 to 20 or
a heteroaromatic group in which the number of ring-constituting atoms is from 5 to 20,
each optionally having a substituent; and
R¹ optionally forms a ring structure by being linked to ring A or ring C via an oxygen atom, a sulfur atom, a substituted or unsubstituted nitrogen atom, Ra or Rc, and
R² optionally forms a ring structure by being linked to ring B or ring C via an oxygen atom, a sulfur atom, a substituted or unsubstituted nitrogen atom, Rb or Rc. 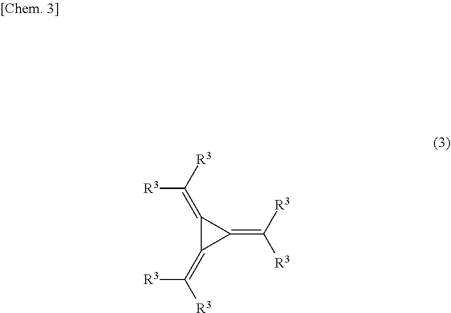
In formula (3),
R³ represents a cyano group, a halogen atom, a fluoroalkyl group, an acyl group, a sulfonyl group, a phosphoryl group, a substituted or unsubstituted aromatic hydrocarbon group or a substituted or unsubstituted aromatic heterocyclic group, independently at each occurrence.

2. The organic electroluminescent element according to claim 1, wherein the organic electroluminescent element satisfies (i) of the conditions (i) and (ii).

3. The organic electroluminescent element according to claim 1, wherein the organic electroluminescent element satisfies (ii) of the conditions (i) and (ii).

4. The organic electroluminescent element according to claim 1, wherein the organic electroluminescent element satisfies both (i) and (ii) of the conditions (i) and (ii).

5. The organic electroluminescent element according to claim 2, wherein in formula (1), at least one of rings A to C is a benzene ring optionally having a substituent.

6. The organic electroluminescent element according to claim 2, wherein in formula (1), rings A to C are ring systems containing a benzene structure optionally having a substituent.

7. The organic electroluminescent element according to claim 2, wherein in formula (1), at least one of Ra, Rb or Rc is a C1 to C4 alkyl group.

8. The organic electroluminescent element according to claim 2, wherein the boron compound represented by formula (1) is selected from the following.

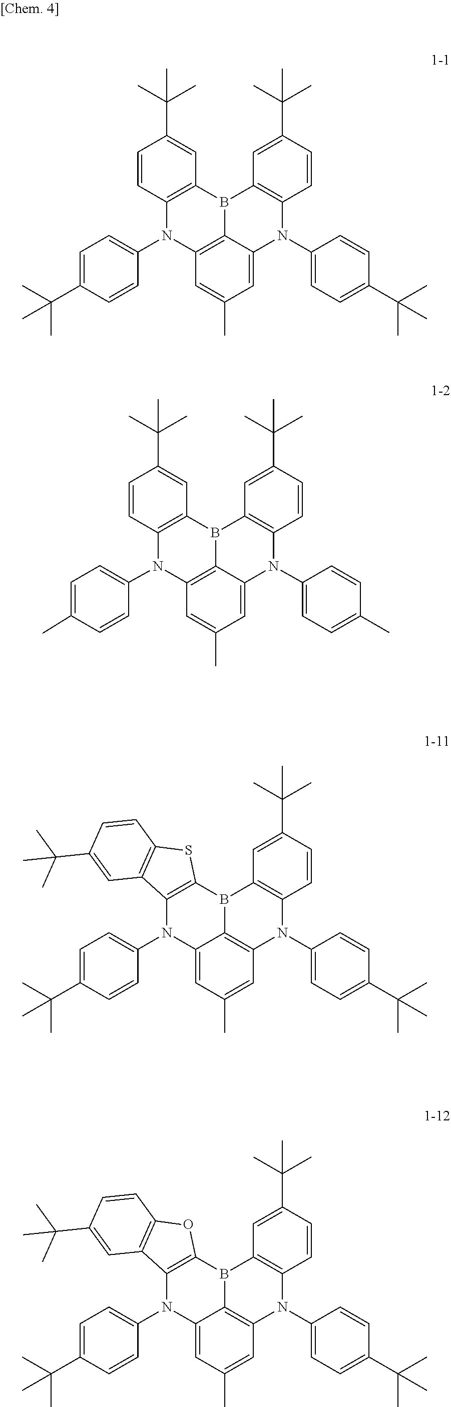

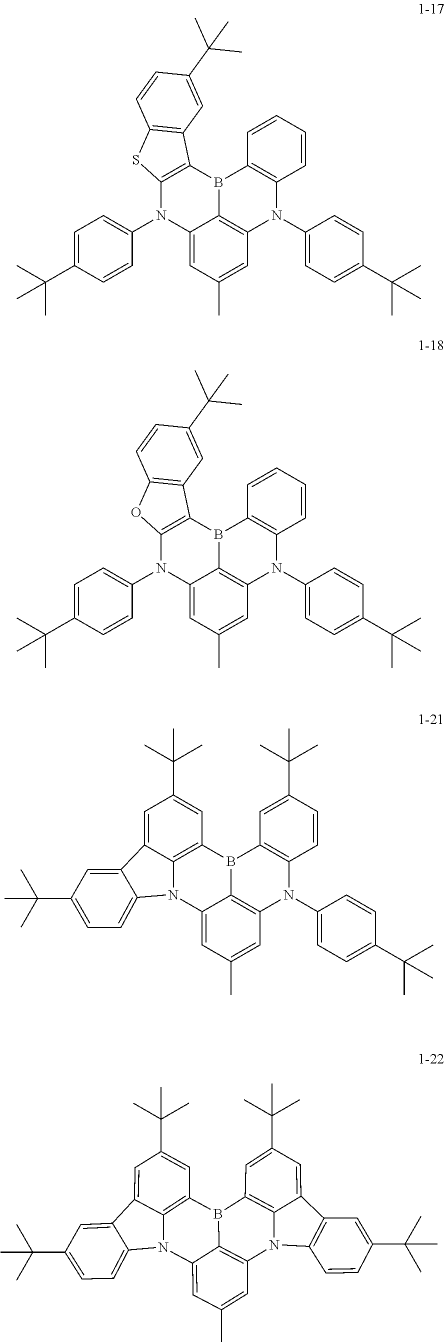

9. The organic electroluminescent element according to claim 2, wherein the boron compound represented by formula (1) is the compound of 1-1 above.

10. The organic electroluminescent element according to claim 3, wherein the hole transport region includes at least a first hole transport layer and
a second hole transport layer disposed between the first hole transport layer and the light-emitting layer, and
the first hole transport layer contains the radialene compound represented by formula (3).

11. The organic electroluminescent element according to claim 3, wherein the radialene compound represented by formula (3) is formula (3-a). 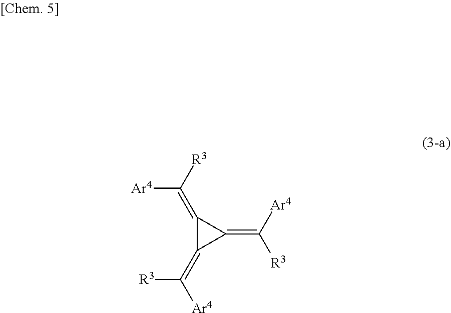
In formula (3-a),
R³ represents a cyano group, a halogen atom, a fluoroalkyl group, an acyl group, a sulfonyl group, a phosphoryl group, a substituted or unsubstituted aromatic hydrocarbon group or a substituted or unsubstituted aromatic heterocyclic group, independently at each occurrence.
Ar⁴ represents a substituted or unsubstituted aromatic hydrocarbon group or a substituted or unsubstituted aromatic heterocyclic group, independently at each occurrence.
Each substituent that a substituted or unsubstituted aromatic hydrocarbon group or substituted or unsubstituted aromatic heterocyclic group represented by R³ and Ar⁴ has is independently selected from
deuterium, a cyano group, a halogen atom, a fluoroalkyl group, an acyl group, a sulfonyl group and a phosphoryl group.

12. The organic electroluminescent element according to claim 3, wherein the radialene compound represented by formula (3) is formula (3-b). 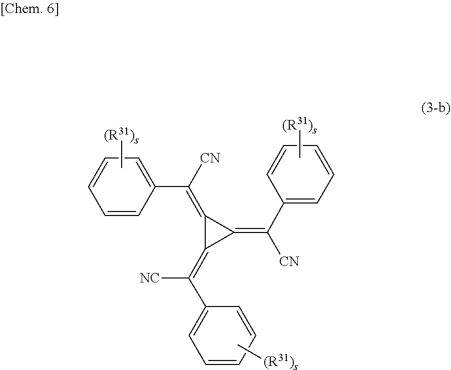
In formula (3-b),
R³¹ is selected from deuterium, a cyano group, a halogen atom, a fluoroalkyl group, an acyl group, a sulfonyl group and a phosphoryl group, independently at each occurrence.
s is an integer of 1 to 5, independently at each occurrence.

13. The organic electroluminescent element according to claim 3, wherein the radialene compound represented by formula (3) is formula 3-1 below.

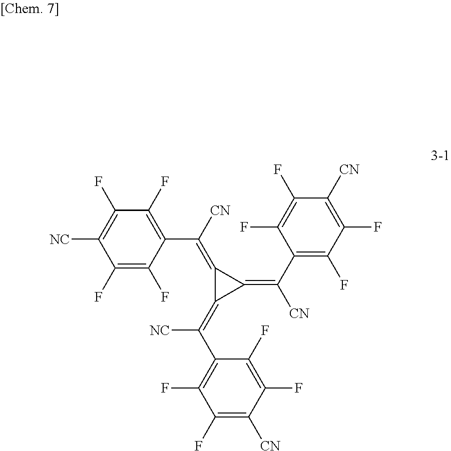 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

14. The organic electroluminescent element according to claim 1, wherein the electron transport region includes at least a first electron transport layer and a second electron transport layer disposed between the first electron transport layer and the negative electrode, and
the second electron transport layer contains the triazine compound represented by formula (2).

15. The organic electroluminescent element according to claim 1, wherein the triazine compound represented by formula (2) is the following.

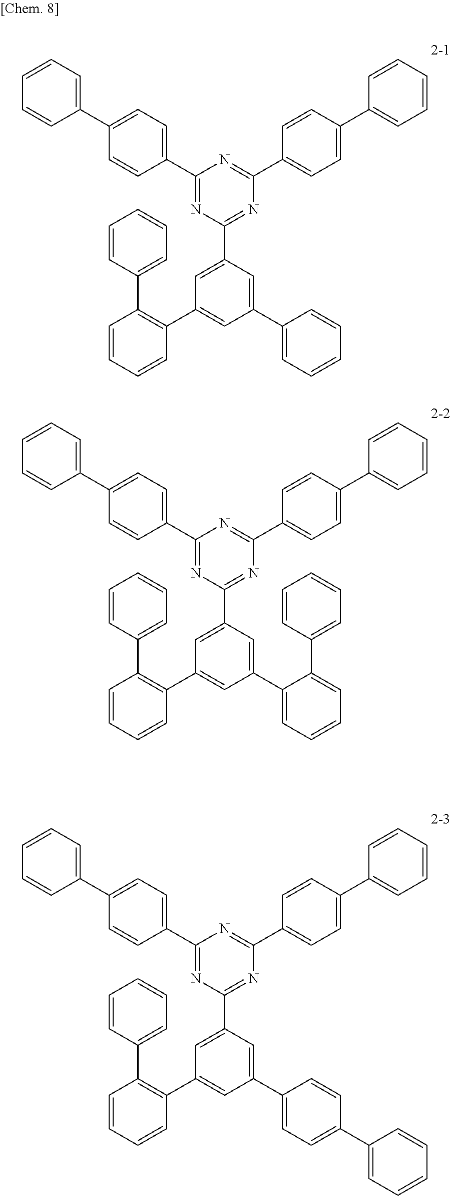 [SMILES: c1ccc(-c2ccc(-c3nc(-c4ccc(-c5ccccc5)cc4)nc(-c4cc(-c5ccccc5)cc(-c5ccccc5-c5ccccc5)c4)n3)cc2)cc1 ; c1ccc(-c2ccc(-c3nc(-c4ccc(-c5ccccc5)cc4)nc(-c4cc(-c5ccccc5-c5ccccc5)cc(-c5ccccc5-c5ccccc5)c4)n3)cc2)cc1 ; c1ccc(-c2ccc(-c3cc(-c4nc(-c5ccc(-c6ccccc6)cc5)nc(-c5ccc(-c6ccccc6)cc5)n4)cc(-c4ccccc4-c4ccccc4)c3)cc2)cc1]

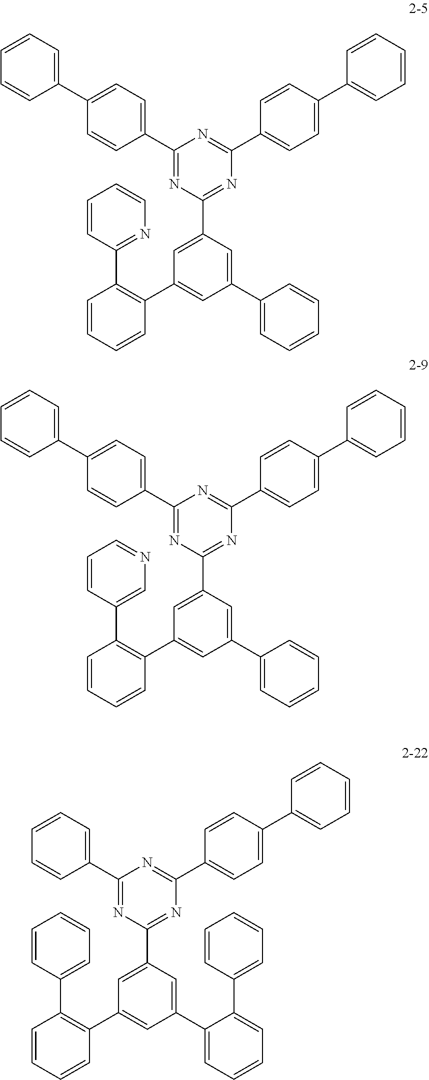 [SMILES: c1ccc(-c2ccc(-c3nc(-c4ccc(-c5ccccc5)cc4)nc(-c4cc(-c5ccccc5)cc(-c5ccccc5-c5ccccn5)c4)n3)cc2)cc1 ; c1ccc(-c2ccc(-c3nc(-c4ccc(-c5ccccc5)cc4)nc(-c4cc(-c5ccccc5)cc(-c5ccccc5-c5cccnc5)c4)n3)cc2)cc1 ; c1ccc(-c2ccc(-c3nc(-c4ccccc4)nc(-c4cc(-c5ccccc5-c5ccccc5)cc(-c5ccccc5-c5ccccc5)c4)n3)cc2)cc1]

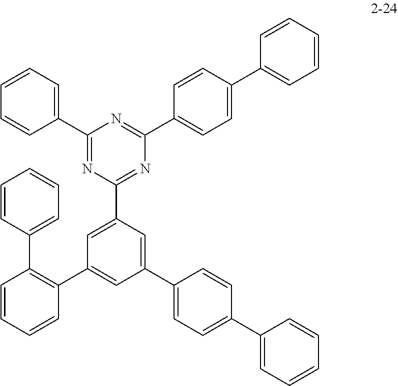 [SMILES: c1ccc(-c2ccc(-c3cc(-c4nc(-c5ccccc5)nc(-c5ccc(-c6ccccc6)cc5)n4)cc(-c4ccccc4-c4ccccc4)c3)cc2)cc1]

16. The organic electroluminescent element according to claim 14, wherein the second electron transport layer contains the compound represented by formula (2) and lithium quinolate (Liq). [chemical structure]
